SEQUENCE LISTING (1 sequence)

SEQ ID NO: 1 - DNA, 27 bp, Artificial Sequence
  Chemically synthesized
aagaacggagaagagattcacgtggta

(19) United States
(12) Patent Application Publication
(10) Pub. No.: US 2023/0151021 A1
(43) Pub. Date: May 18, 2023

(54) Compounds and Compositions for Treating Hematologic Malignancies

(71) Applicants: THE TRUSTEES OF THE UNIVERSITY OF PENNSYLVANIA, Philadelphia, PA (US); BARUCH S. BLUMBERG INSTITUTE, Doylestown, PA (US)

(72) Inventors: Erle S. Robertson, Wynnewood, PA (US); Yonggang Pei, Philadelphia, PA (US); Yanming Du, Cheshire, CT (US)

(21) Appl. No.: 17/920,663

(22) PCT Filed: Apr. 27, 2021
(86) PCT No.: PCT/US21/29275

§ 371 Date: Oct. 21, 2022

Related U.S. Application Data
(60) Provisional application No. 63/016,054, filed on Apr. 27, 2020.

(51) Int. Cl. C07D 493/08; A61P 35/02

(52) U.S. Cl. CPC C07D 493/08; A61P 35/02

(57) ABSTRACT

The present disclosure relates, in part, to certain compounds comprising brusatol derivatives, or a pharmaceutically acceptable salt, solvate, or polymorph thereof, which are useful for the treatment of hematological malignancies, including but not limited to leukemia and lymphoma. In certain embodiments, the present disclosure relates to pharmaceutical compositions of the compounds of the present disclosure.

### CROSS-REFERENCE TO RELATED APPLICATIONS

This application claims to priority under 35 U.S.C. § 119(e) to U.S. Provisional Patent Application No. 63/016,054, filed Apr. 27, 2020, which is hereby incorporated herein by reference in its entirety.

### STATEMENT REGARDING FEDERALLY SPONSORED RESEARCH OR DEVELOPMENT

This invention was made with government support under Grant Nos. CA016520, CA171979, CA174439, CA-177423 awarded by the National Institutes of Health. The government has certain rights in the invention.

### SEQUENCE LISTING

The ASCII text file named “046483-7270WO1(02541) Sequence Listing” created on Apr. 27, 2021, comprising 0.6 Kbytes, is hereby incorporated by reference in its entirety.

### BACKGROUND OF THE INVENTION

Phosphoinositide 3-kinases (PI3Ks) are involved in an extraordinarily broad range of physiological processes, and elevated levels are typically identified as a critical hallmark of a number of human cancers. Development of small-molecule inhibitors selectively targeting PI3Ks is an extremely promising strategy as the PI3Ks pathway is associated with many features linked to the tumorigenic process. Three PI3Ks inhibitors, Idelalisib, Copanlisib, Duvelisib, have been approved by the FDA for treating relapsed or refractory chronic lymphocytic leukemia (CLL), small lymphocytic lymphoma (SLL), or indolent lymphoma. Another PI3Kγ inhibitor, IPI-549, which was shown to overcome resistance to immune checkpoint blockade (ICB), by reshaping the tumor microenvironment to promote cytotoxic-T-lymphocyte-associated tumor regression, is also progressing in clinical trials. These inhibitors demonstrate the potential of targeting the PI3K protein family members for treatment of numerous types of solid cancers. However, one of the major obstacles limiting their success in clinical trials is the resistance and poor tolerability to PI3Ks inhibitors. Therefore, proposed solutions for improving their impacts in solid cancer treatment include the development of more specific PI3Ks inhibitors, and to ameliorate the associated toxicities of these inhibitors. Ongoing clinical trials of PI3K targeted therapies in combination with other therapies should provide additional evidence to support PI3Ks inhibitors as important therapeutic agents for treatment of human cancer.

The Quassinoid family of natural compounds includes over 150 members and has been known for decades to have anti-cancer activities. Brusatol, obtained from the plant Brucea javanica, has demonstrated anti-cancer activity, but it has not been tested in human patients. Previous studies showed that Brusatol could be used as an adjuvant chemotherapeutic drug by inhibiting the Nrf2 signaling pathway, however, the results indicated that the inhibition was directly through targeting the translation of cap-dependent and cap-independent proteins rather than Nrf2. Another study showed that Brusatol functions as a protein synthesis inhibitor in a manner independent of Nrf2. Therefore, the mechanism of its anti-cancer activities is yet to be fully understood, although many studies recognized Brusatol as an Nrf2 specific inhibitor. This hampers the development and use of these Quassinoids family members for treatment of hematologic malignancies as well as other solid cancers.

There is a need in the art for novel compounds and compositions that can be used to treat cancer. The present disclosure addresses this need.

### BRIEF SUMMARY OF THE INVENTION

In one aspect, the present disclosure relates to certain compounds, including but not limited to compounds of formula (I), or a pharmaceutically acceptable salt, solvate, or polymorph thereof:

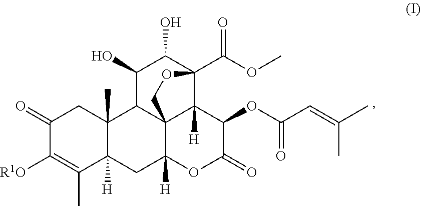

wherein the various substituents in the compounds of formula (I) are defined elsewhere herein. The present disclosure further relates to pharmaceutical compositions comprising the compounds of the present disclosure.

In another aspect, the compounds and compositions of the present disclosure are useful for treating a hematological malignancy in a subject in need thereof. In certain embodiments, the hematological malignancy is selected from the group consisting of leukemia and lymphoma.

### BRIEF DESCRIPTION OF THE DRAWINGS

The following detailed description of illustrative embodiments of the invention will be better understood when read in conjunction with the appended drawings. For the purpose of illustrating the invention, certain illustrative embodiments are shown in the drawings. It should be understood, however, that the invention is not limited to the precise arrangements and instrumentalities of the embodiments shown in the drawings.

FIGS. 1A-1F: The Quassinoid compound Brusatol inhibits viability of hematologic malignancy derived cells. FIGS. 1A-1B: A serial number of Raji (FIG. 1A) or LCL1 (FIG. 1B) cells were incubated with 100 nM Brusatol for 72 hours. Cell viability was determined by detecting luminescence. Untreated cells were set as a negative control. Results are the mean±standard error of the duplicates. *p<0.05 and ****p<0.0001 show the significant differences between Brusatol-treated cells and the control group. FIG. 1C: 5000 Raji or LCL1 cells were incubated with a serial concentration of Brusatol for 72 hours. Cell viability was determined by detecting luminescence and relative luminescence was shown by comparing to untreated samples. FIG. 1D: 5000 indicated cells were untreated or treated with 100 nM Brusatol. After 72 hours of incubation, cell viability was determined and shown. ALL, acute lymphoblastic leukemia; BL, Burkitt's lymphoma; DLBCL, diffuse large B-cell lymphoma; MCL, mantle cell lymphoma; AML, acute myeloid leukemia; MM, multiple myeloma; LCLs, lymphoblastoid cell lines. Results are the mean±standard error of the duplicates. ***p<0.001 and ****p<0.0001 show the significant differences as compared to the corresponding control group. FIG. 1E: Indicated PDXs were cultured in 96-well plates and treated with different concentrations of Brusatol for 72 hours. Cell viability was monitored by testing luminescence. FL (PDX-129), B-cell Follicular lymphoma; DLBCL (PDX-223), Diffuse large B cell lymphoma; BL (PDX-255), Burkitt's lymphoma. FIG. 1F: LCL1 cells were untreated or treated with Brusatol (40 nM, 100 nM) for 72 hours. Then cells were fixed, stained, and analyzed with flow cytometry for cell cycle assay. The subG1 population in cells was labeled as the percentage.

FIGS. 2A-2B: RNA-seq analysis of Brusatol-treated cells. FIG. 2A: Microscopic pictures show the normal status of tested cells. FIG. 2B: Heatmap summarized the up-regulated and down-regulated genes in Brusatol-treated LCL1 cells.

FIGS. 3A-3I: Brusatol specifically targets the PI3K/AKT signaling pathway. FIG. 3A: RNA-Seq analysis was performed in Brusatol-treated LCL1 cells. The heatmap illustrates 12 genes with the most significant differences after Brusatol treatment. FIG. 3B: Signaling pathway analysis of RNA-Seq data showed the percentages of regulated genes in these indicated pathways. The numbers above the bar charts described the total genes involved in these different pathways. FIG. 3C: Upstream regulator analysis in the IPA program showed PI3K family members are the important upstream factors. FIG. 3D: Chemical structures of the parent Brusatol and three biotin-conjugated Brusatol analogs were shown. The synthesized C-3-biotinylated Brusatol derivatives (51048, 51052) were set as the experimental samples (+), while a C-21 biotin conjugate (51046) was set as a negative control (−). FIG. 3E: Correlation of mass spectrometry data and upstream regulator data were conducted using the IPA program. FIG. 3F: Real-time PCR was performed in untreated or Brusatol-treated Raji cells to verify the mRNA expression of upstream regulators (PI3K family, TP73, JUP) associated genes. The results from three independent experiments were shown. Results are the mean±standard error of the samples (n=3). **p<0.01 and ****p<0.0001 show the significant differences as compared to the control group. FIG. 3G: Real-time PCR was performed to determine the mRNA expression of PI3K/AKT signaling members in LCL1 cells after Brusatol treatment. Results are the mean±standard error of the samples (n=3). *p<0.05 shows significant differences as compared to the control group. FIGS. 3H-3I: Western blot analysis was conducted to show the protein expression of PI3K/AKT signaling factors in Raji (FIG. 3H) or LCL1 (FIG. 3I) cells after Brusatol treatment. The PI3Kγ band was labeled by an asterisk. Two bands of GSK3 were GSK3α (51 kD) and GSK3β (47 kD), respectively.

FIG. 4: Biotin-conjugated Brusatol compounds exhibit inhibitory effects. Chemical structures of synthesized biotin-conjugated compounds were shown.

FIGS. 5A-5F: PI3K family of proteins is crucial for Brusatol-mediated inhibitory effects. FIG. 5A: IC₅₀ assays of Brusatol were determined in the indicated cancer cells. All of the cells were treated for 72 hours. FIG. 5B: The indicated Brusatol-less sensitive or -more sensitive cells were treated with 100 nM Brusatol for indicated periods (0, 24 hours, 48 hours, 72 hours), and cell viability was examined by detecting luminescence. Results are the mean±standard error of the duplicates. ****p<0.0001 shows the significant differences between the indicated groups. NS, not significant. FIGS. 5C-5D: Brusatol-less sensitive (FIG. 5C) or -more sensitive (FIG. 5D) cells were treated with 100 nM Brusatol for different periods (0, 24 hours, 48 hours). Then cells were fixed and stained with PI. Flow cytometry assays were performed to determine cell cycle progression. The subG1 population in cells was labeled as the percentage. FIG. 5E: The endogenous expression of PI3K/AKT signaling protein in these cell lines, including sets of Brusatol-less sensitive and -more sensitive cells, was detected with western blot. FIG. 5F: The described cells were incubated with Brusatol (0, 50 nM, 100 nM) for different periods (0, 12 hours, 24 hours). Western blot analysis was performed to determine the expression of PI3K/AKT regulated signaling proteins in Brusatol-less sensitive cells set (HL-60, K562) and Brusatol-more sensitive cells set (Raji, SU-DHL-4).

FIGS. 6A-6H: The Quassinoid family member Brusatol can directly target the PI3Kγ isoform. FIG. 6A: IC₅₀ of Brusatol was determined in these NPC cell lines (C17, NPC43, and NPC53) and Brusatol-sensitive SU-DHL-4 cells at 72 hours post-treatment. FIG. 6B: These cell lines were harvested and detected the endogenous expressions of indicated proteins with western blot analysis. FIG. 6C: The lysates of SU-DHL-4 cells were incubated with biotin-conjugated 51048 compound together in the absence or presence of Brusatol, then the immunoprecipitated complex was detected with western blot. FIG. 6D: In vitro expressed and purified GST-tagged PI3Kγ protein was incubated with 0, 300 μM, or 500 μM biotin-conjugated 51052 compound as well as Dynabeads M-280 Streptavidin. Then the binding protein was examined with western blot analysis. FIG. 6E: A schematic diagram shows the target site (SEQ ID NO:1) of PIK3CG genes using the CRISPR/Cas9 system. FIG. 6F: A Surveyor mutation detection assay was performed to verify whether the PIK3CG gene was mutated in knock-out (KO) Raji cells. The control cell line (sgVec) by transfecting empty plasmids were used as control. The yellow arrows indicated the truncated fragments. FIG. 6G: The indicated proteins were detected in knock-out Raji cells by western blot analysis. FIG. 6H: Knock-out Raji cells were untreated or treated with 100 nM of Brusatol for 72 hours, then cells were harvested and determined the expressions of PI3K/AKT associated proteins with western blot.

FIGS. 7A-7I: Developed Brusatol analogs show inhibitory effects in vitro and in vivo. FIGS. 7A-7B: Chemical structures of representative Brusatol analogs with different modifications were shown. FIG. 7C: The compound 1 was shown as an inactivated drug. FIG. 7D: MOLM14 cells were treated with 100 nM Brusatol for different times (0, 24 hours, 48 hours, 72 hours) and cell viability was examined by detecting luminescence activity. Results are the mean±standard error of the duplicates. ****p<0.0001 shows the significant differences between the indicated groups. FIG. 7E: MOLM14 cells were treated with 100 nM Brusatol for indicated times (0, 24 hours, 48 hours) and cell cycle assay was determined with flow cytometry. The subG1 population in cells was labeled as the percentage. FIG. 7F: The expressions of PI3K associated proteins were detected in Raji, MOLM14, and SU-DHL-4 cells with western blot. FIG. 7G: The microscopic examination of these tumors from the dissected mice was shown. FIG. 7H: Mice weight was monitored after Brusatol or its analogs treatment in vivo. FIG. 7I: Total RNAs were extracted from the xenografts, and Real-time PCR analysis was performed to detect PIK3CG and GSK3B expression. Results are the mean±standard error of the samples (n=3). *p<0.05 and **p<0.01 show the significant differences as compared to the control group.

FIGS. 8A-8E: Development of novel Brusatol analogs with great efficacy in vitro and in vivo. FIG. 8A: Multiple hematopoietic malignant cell lines were untreated or treated with 100 nM of compound 14, 15, 26, 31, or 1. Cell viability was determined after 72 hours by detecting luminescence. ALL, acute lymphoblastic leukemia; BL, Burkitt's lymphoma; DLBCL, diffuse large B-cell lymphoma; MCL, mantle cell lymphoma; AML, acute myeloid leukemia; MM, multiple myeloma; LCLs, lymphoblastoid cell lines. Results are the mean standard error of the duplicates. FIG. 8B: Chemical structures of these tested Brusatol analogs were shown. FIG. 8C: Raji cells were treated with different concentrations of these analogs for 72 hours to determine their IC₅₀ using cell viability assays. FIG. 8D: Tumor sizes were monitored in the mice after treatment of the indicated compounds. Results are the mean±standard error of the mice (n>5). FIG. 8E: Tumor size at 12-day post-treatment with different analogs was highlighted. Results are the mean±standard error of the mice (n>5). **p<0.01 shows significant differences as compared to the control group.

FIGS. 9A-9D show the identification of active brusatol conjugates. FIG. 9A: generic structure of brusatol analogs. FIG. 9B: structure of individual conjugates. FIG. 9C: activities of analogs on Raji cell lines. FIG. 9D: activities of selected compounds on five additional cell lines (MOLT-4, SU-DHL-6, SU-DHL-10, RPMI-8226, and LCL1).

FIGS. 10A-10D provide SAR studies around compounds 26 and 31. FIG. 10A: generic structure of brusatol analogs. FIG. 10B: structure of individual conjugates. FIG. 10C: activities of analogs on Raji cell lines. FIG. 10D: activities of selected compounds on five additional cell lines (MOLT-4, SU-DHL-6, SU-DHL-10, RMPI-8226, and LCL1).

FIGS. 11A-11D provide the activities of non-cleavable brusatol analogs. FIG. 11A: generic structure of brusatol analogs. FIG. 11B: structure of individual conjugates. FIG. 11C: activities of analogs on Raji cell lines. FIG. 11D: activities of selected compounds on five additional cell lines (MOLT-4, SU-DHL-6, SU-DHL-10, RPMI-8226, and LCL1).

FIG. 12 provides a scheme showing the metabolites of 26. MetID study of 26 showed that 26 was the major fraction after 60 min of incubation in mouse microsomes.

FIGS. 13A-13I: One novel Brusatol analog is a potential PI3K inhibitor with minimal toxicity. FIGS. 13A-13C: IC₅₀ assays were performed by treating PBMC (FIG. 13A), normal T-cells (FIG. 13B), or normal B-cells (FIG. 13C) with Brusatol analogs and other PI3K inhibitors. PBMC and normal T-cells were treated for 72 hours, and normal B-cells were treated for 24 hours. FIGS. 13D-13F: IC₅₀ of Brusatol analogs and PI3K inhibitors was investigated in SU-DHL-4 (FIG. 13D), MOLM14 (FIG. 13E), and Raji (FIG. 13F) cells. All of these cells were treated for 72 hours. FIGS. 13G-13H: HL-60 (FIG. 13G) and Raji (FIG. 13H) cells were incubated with analog 15 or 26 (0, 50 nM, 100 nM) for indicated times (0, 12 hours, 24 hours). Western blot analysis was performed to determine the expression of PI3K/AKT regulated signaling proteins. FIG. 13I: NOD/SCID mice (4 per group) were intra-peritoneally injected 10 mg/kg of indicated compounds (Brusatol, 15, and 26) every other day. The survival rate was monitored and demonstrated to show their toxicity in vivo. Results are the survival rate of the mice (n=4). *p<0.05 and **p<0.01 show the significant differences between the indicated groups. NS, not significant.

FIG. 14 provides a toxicity evaluation of 15 and 26 compared to brusatol in mice under 5 mg/kg dose; non-significant (NS).

FIG. 15A: Blood concentration vs time profile for Brusatol after 3 mg/kg IV in CD1 mouse. FIG. 15B: Blood concentration vs time profile for Brusatol after 10 mg/kg PO in CD1 mouse. FIG. 15C: Mean blood concentration vs time profile for Brusatol after IV and PO in CD1 mouse.

FIG. 16A: Blood concentration vs time profile for 15 after 3 mg/kg IV in CD1 mouse. FIG. 16B: Mean blood concentration vs time profile for 15 after IV and PO in CD1 mouse.

FIG. 17A: Blood concentration vs time profile for Brusatol (Met) after 3 mg/kg IV of 26 in CD1 mouse. FIG. 17B: Blood concentration vs time profile for Brusatol (Met) after 10 mg/kg PO of 26 in CD1 mouse. FIG. 17C: Mean blood concentration vs time profile for Brusatol (Met) after IV and PO of 26 in CD1 mouse.

### DETAILED DESCRIPTION OF THE INVENTION

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described. As used herein, each of the following terms has the meaning associated with it in this section.

Generally, the nomenclature used herein and the laboratory procedures in cell culture, molecular genetics, pharmacology and organic chemistry are those well-known and commonly employed in the art.

Standard techniques are used for biochemical and/or biological manipulations. The techniques and procedures are generally performed according to conventional methods in the art and various general references (e.g., Sambrook and Russell, 2012, Molecular Cloning, A Laboratory Approach, Cold Spring Harbor Press, Cold Spring Harbor, N.Y., and Ausubel et al., 2002, Current Protocols in Molecular Biology, John Wiley & Sons, NY), which are provided throughout this document.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

A disease or disorder is “alleviated” if the severity or frequency of at least one sign or symptom of the disease or disorder experienced by a patient is reduced.

The term “amine” as used herein refers to primary, secondary, and tertiary amines having, e.g., the formula N(group)₃ wherein each group can independently be H or non-H, such as alkyl, aryl, and the like. Amines include but are not limited to R—NH₂, for example, alkylamines, arylamines, alkylarylamines; R₂NH wherein each R is independently selected, such as dialkylamines, diarylamines, aralkylamines, heterocyclylamines and the like; and R₃N wherein each R is independently selected, such as trialkylamines, dialkylarylamines, alkyldiarylamines, triarylamines, and the like. The term “amine” also includes ammonium ions as used herein.

The term “aminoalkyl” as used herein refers to an alkyl group substituted at any one position with an amine as defined herein.

As used herein, the terms “analog,” “analogue,” or “derivative” are meant to refer to a chemical compound or molecule made from a parent compound or molecule by one or more chemical reactions. As such, an analog can be a structure having a structure similar to that of the small molecule inhibitors described herein or can be based on a scaffold of a small molecule inhibitor described herein, but differing from it in respect to certain components or structural makeup, which may have a similar or opposite action metabolically.

As used herein, the term “binding” refers to the adherence of molecules to one another, such as, but not limited to, enzymes to substrates, antibodies to antigens, DNA strands to their complementary strands. Binding occurs because the shape and chemical nature of parts of the molecule surfaces are complementary. A common metaphor is the “lock-and-key” used to describe how enzymes fit around their substrate.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

An “effective amount” or “therapeutically effective amount” of a compound is that amount of compound sufficient to provide a beneficial effect to the subject to which the compound is administered. An “effective amount” of a delivery vehicle is that amount sufficient to effectively bind or deliver a compound.

The term “haloalkyl” group, as used herein, includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, and the like.

The phrase “inhibit,” as used herein, means to reduce a molecule, a reaction, an interaction, a gene, an mRNA, and/or a protein's expression, stability, function or activity by a measurable amount or to prevent entirely. Inhibitors are compounds that, e.g., bind to, partially or totally block stimulation, decrease, prevent, delay activation, inactivate, desensitize, or down regulate a protein, a gene, and an mRNA stability, expression, function and activity, e.g., antagonists.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the patient. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, Pa.), which is incorporated herein by reference.

As used herein, the language “pharmaceutically acceptable salt” or “therapeutically acceptable salt” refers to a salt of the administered compounds prepared from pharmaceutically acceptable non-toxic acids, including inorganic acids or bases, organic acids or bases, solvates, hydrates, or clathrates thereof.

The terms “pharmaceutically effective amount” and “effective amount” refer to a nontoxic but sufficient amount of an agent to provide the desired biological result. That result can be reduction and/or alleviation of the signs, symptoms, or causes of a disease or disorder, or any other desired alteration of a biological system. An appropriate effective amount in any individual case may be determined by one of ordinary skill in the art using routine experimentation.

As used herein, the terms “polypeptide,” “protein” and “peptide” are used interchangeably and refer to a polymer composed of amino acid residues, related naturally occurring structural variants, and synthetic non-naturally occurring analogs thereof linked via peptide bonds. Synthetic polypeptides can be synthesized, for example, using an automated polypeptide synthesizer.

By the term “specifically binds,” as used herein, is meant a molecule, such as an antibody, which recognizes and binds to another molecule or feature, but does not substantially recognize or bind other molecules or features in a sample.

A “therapeutic” treatment is a treatment administered to a subject who exhibits signs of pathology, for the purpose of diminishing or eliminating those signs.

As used herein, the term “therapeutically effective amount” is an amount of a compound of the invention, that when administered to a patient, ameliorates a symptom of the disease or disorder. The amount of a compound of the invention that constitutes a “therapeutically effective amount” will vary depending on the compound, the disease state and its severity, the age of the patient to be treated, and the like. The therapeutically effective amount can be determined routinely by one of ordinary skill in the art having regard to his own knowledge and to this disclosure.

As used herein, “treating a disease or disorder” means reducing the frequency with which a symptom of the disease or disorder is experienced by a patient. Disease and disorder are used interchangeably herein.

As used herein, the term “treatment” or “treating” encompasses prophylaxis and/or therapy. Accordingly the compositions and methods of the present invention are not limited to therapeutic applications and can be used in prophylaxis ones. Therefore “treating” or “treatment” of a state, disorder or condition includes: (i) preventing or delaying the appearance of clinical symptoms of the state, disorder or condition developing in a subject that may be afflicted with or predisposed to the state, disorder or condition but does not yet experience or display clinical or subclinical symptoms of the state, disorder or condition, (ii) inhibiting the state, disorder or condition, i.e., arresting or reducing the development of the disease or at least one clinical or subclinical symptom thereof, or (iii) relieving the disease, i.e. causing regression of the state, disorder or condition or at least one of its clinical or subclinical symptoms.

As used herein, the term “wild-type” refers to the genotype and phenotype that is characteristic of most of the members of a species occurring naturally and contrasting with the genotype and phenotype of a mutant.

As used herein, the term “alkyl,” by itself or as part of another substituent means, unless otherwise stated, a straight or branched chain hydrocarbon having the number of carbon atoms designated (i.e., C₁-C₁₀ means one to ten carbon atoms) and includes straight, branched chain, or cyclic substituent groups. Examples include methyl, ethyl, propyl, isopropyl, butyl, isobutyl, tert-butyl, pentyl, neopentyl, hexyl, and cyclopropylmethyl. Certain specific examples include (C₁-C₆)alkyl, such as, but not limited to, ethyl, methyl, isopropyl, isobutyl, n-pentyl, n-hexyl and cyclopropylmethyl.

As used herein, the term “cycloalkyl,” by itself or as part of another substituent means, unless otherwise stated, a cyclic chain hydrocarbon having the number of carbon atoms designated (i.e., C₃-C₆ means a cyclic group comprising a ring group consisting of three to six carbon atoms) and includes straight, branched chain or cyclic substituent groups. Examples include cyclopropyl, cyclobutyl, cyclopentyl, cyclohexyl, cycloheptyl, and cyclooctyl. Certain specific examples include (C₃-C₆)cycloalkyl, such as, but not limited to, cyclopropyl, cyclobutyl, cyclopentyl and cyclohexyl.

As used herein, the term “substituted alkyl” or “substituted cycloalkyl” means alkyl or cycloalkyl, as defined above, substituted by one, two or three substituents selected from the group consisting of halogen, —OH, alkoxy, tetrahydro-2-H-pyranyl, —NH₂, —N(CH₃)₂, (1-methyl-imidazol-2-yl), pyridin-2-yl, pyridin-3-yl, —C(═O)OH, trifluoromethyl, —C≡N, —C(═O)O(C₁-C₄)alkyl, —C(═O)NH₂, —C(═O)NH(C₁-C₄)alkyl, —C(═O)N((C₁-C₄)alkyl)₂, —SO₂NH₂, —C(═NH)NH₂, and —NO₂, advantageously containing one or two substituents selected from halogen, —OH, alkoxy, —NH₂, trifluoromethyl, —N(CH₃)₂, and —C(═O)OH, more advantageously selected from halogen, alkoxy and —OH. Examples of substituted alkyls include, but are not limited to, 2,2-difluoropropyl, 2-carboxycyclopentyl and 3-chloropropyl.

As used herein, the term “alkoxy” employed alone or in combination with other terms means, unless otherwise stated, an alkyl group having the designated number of carbon atoms, as defined above, connected to the rest of the molecule via an oxygen atom, such as, for example, methoxy, ethoxy, 1-propoxy, 2-propoxy (isopropoxy) and the higher homologs and isomers. In certain embodiments, alkoxy includes (C₁-C₃)alkoxy, such as, but not limited to, ethoxy and methoxy.

As used herein, the term “halo” or “halogen” alone or as part of another substituent means, unless otherwise stated, a fluorine, chlorine, bromine, or iodine atom, advantageously, fluorine, chlorine, or bromine, more advantageously, fluorine or chlorine.

As used herein, the term “heteroalkyl” by itself or in combination with another term means, unless otherwise stated, a stable straight or branched chain alkyl group consisting of the stated number of carbon atoms and one or two heteroatoms selected from the group consisting of O, N, and S, and wherein the nitrogen and sulfur atoms may be optionally oxidized and the nitrogen heteroatom may be optionally quaternized. The heteroatom(s) may be placed at any position of the heteroalkyl group, including between the rest of the heteroalkyl group and the fragment to which it is attached, as well as attached to the most distal carbon atom in the heteroalkyl group. Examples include: —O—CH₂—CH₂—CH₃, —CH₂—CH₂—CH₂—OH, —CH₂—CH₂—NH—CH₃, —CH₂—S—CH₂—CH₃, and —CH₂CH₂—S(═O)—CH₃. Up to two heteroatoms may be consecutive, such as, for example, —CH₂—NH—OCH₃, or —CH₂—CH₂—S—S—CH₃.

As used herein, the term “aromatic” refers to a carbocycle or heterocycle with one or more polyunsaturated rings and having aromatic character, i.e. having (4n+2) delocalized π (pi) electrons, where n is an integer.

As used herein, the term “aryl,” employed alone or in combination with other terms, means, unless otherwise stated, a carbocyclic aromatic system containing one or more rings (typically one, two or three rings) wherein such rings may be attached together in a pendent manner, such as a biphenyl, or may be fused, such as naphthalene. Examples include phenyl, anthracyl, and naphthyl. In certain embodiments, aryl includes phenyl and naphthyl, in particular, phenyl.

As used herein, the term “heterocycle” or “heterocyclyl” or “heterocyclic” by itself or as part of another substituent means, unless otherwise stated, an unsubstituted or substituted, stable, mono- or multi-cyclic heterocyclic ring system that consists of carbon atoms and at least one heteroatom selected from the group consisting of N, O, and S, and wherein the nitrogen and sulfur heteroatoms may be optionally oxidized, and the nitrogen atom may be optionally quaternized. The heterocyclic system may be attached, unless otherwise stated, at any heteroatom or carbon atom that affords a stable structure. A heterocycle may be aromatic or non-aromatic in nature. In certain embodiments, the heterocycle is a heteroaryl.

As used herein, the term “heteroaryl” or “heteroaromatic” refers to a heterocycle having aromatic character. A polycyclic heteroaryl may include one or more rings that are partially saturated. Examples include tetrahydroquinoline and 2,3-dihydrobenzofuryl.

Examples of non-aromatic heterocycles include monocyclic groups such as aziridine, oxirane, thiirane, azetidine, oxetane, thietane, pyrrolidine, pyrroline, imidazoline, pyrazolidine, dioxolane, sulfolane, 2,3-dihydrofuran, 2,5-dihydrofuran, tetrahydrofuran, thiophane, piperidine, 1,2,3,6-tetrahydropyridine, 1,4-dihydropyridine, piperazine, morpholine, thiomorpholine, pyran, 2,3-dihydropyran, tetrahydropyran, 1,4-dioxane, 1,3-dioxane, homopiperazine, homopiperidine, 1,3-dioxepane, 4,7-dihydro-1,3-dioxepin and hexamethyleneoxide.

Examples of heteroaryl groups include pyridyl, pyrazinyl, pyrimidinyl (such as, but not limited to, 2- and 4-pyrimidinyl), pyridazinyl, thienyl, furyl, pyrrolyl, imidazolyl, thiazolyl, oxazolyl, pyrazolyl, isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl.

Examples of polycyclic heterocycles include indolyl (such as, but not limited to, 3-, 4-, 5-, 6- and 7-indolyl), indolinyl, quinolyl, tetrahydroquinolyl, isoquinolyl (such as, but not limited to, 1- and 5-isoquinolyl), 1,2,3,4-tetrahydroisoquinolyl, cinnolinyl, quinoxalinyl (such as, but not limited to, 2- and 5-quinoxalinyl), quinazolinyl, phthalazinyl, 1,8-naphthyridinyl, 1,4-benzodioxanyl, coumarin, dihydrocoumarin, 1,5-naphthyridinyl, benzofuryl (such as, but not limited to, 3-, 4-, 5-, 6- and 7-benzofuryl), 2,3-dihydrobenzofuryl, 1,2-benzisoxazolyl, benzothienyl (such as, but not limited to, 3-, 4-, 5-, 6-, and 7-benzothienyl), benzoxazolyl, benzothiazolyl (such as, but not limited to, 2-benzothiazolyl and 5-benzothiazolyl), purinyl, benzimidazolyl, benztriazolyl, thioxanthinyl, carbazolyl, carbolinyl, acridinyl, pyrrolizidinyl, and quinolizidinyl.

The aforementioned listing of heterocyclyl and heteroaryl moieties is intended to be representative and not limiting.

As used herein, the term “substituted” means that an atom or group of atoms has replaced hydrogen as the substituent attached to another group.

For aryl and heterocyclyl groups, the term “substituted” as applied to the rings of these groups refers to any level of substitution, namely mono-, di-, tri-, tetra-, or penta-substitution, where such substitution is permitted. The substituents are independently selected, and substitution may be at any chemically accessible position. In certain embodiments, the substituents vary in number between one and four. In other embodiments, the substituents vary in number between one and three. In yet another embodiments, the substituents vary in number between one and two. In yet another embodiments, the substituents are independently selected from the group consisting of C₁₋₆ alkyl, —OH, C₁₋₆ alkoxy, halo, amino, acetamido and nitro. As used herein, where a substituent is an alkyl or alkoxy group, the carbon chain may be branched, straight or cyclic, in particular, straight.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The Quassinoid family includes more than 150 compounds that are extracted from the Simaroubaceae plant family. Many Quassinoid members show anti-inflammatory and anti-cancer activities, but the exact mechanism of their actions requires further investigation. Several studies showed that Brusatol induced cell differentiation was associated with MYC down-regulation, and NF-κB activation. Additionally, the toxicity of Quassinoid members has been a major obstacle limiting their applications as anti-cancer drugs in clinical trials. However, modifications of these natural compounds have potential value for the development of therapeutic agents for targeted cancer treatment.

To develop Brusatol as a therapeutic drug for treating hematologic malignancies in early phase clinical trials, a series of Brusatol analogs were strategically synthesized and their activities examined on a broad range of hematologic malignancy derived cell lines. These analogs modified at the C-3 position demonstrated significant inhibition of these cancer cells. Moreover, when compared to the commercially available PI3K inhibitors, these analogs also exhibited comparable cytotoxicity. Notably, analogs 15 and 26 exhibited similar levels of inhibition to Brusatol in xenografts model in vivo. While it is not presently known that the anti-cancer mechanisms of 15 and 26 are similar to the parent Brusatol, both significantly inhibited the PI3K/AKT pathway with specific effects on cells with increased PI3Kγ levels. Furthermore, analog 26 showed significantly reduced toxicity in vivo than Brusatol and analog 15. The data thus demonstrate that the novel analog 26 forms the basis of a platform for the development of targeted small molecule therapeutics for treatment of EBV-positive, and hematologic malignancies in early phase clinical trials with comparative efficacy to current PI3K inhibitors in terms of their potency, but with specificity for PI3Kγ. This adds to their precision in targeting hematologic malignancies with dysregulated PI3Kγ levels.

### Compositions

Without meaning to be limited by theory, the invention is based in part on the unexpected discovery that certain compounds, in some cases derived by modifying the structure of Brusatol, are useful in the treatment of cancer, in particular hematological malignancies.

In one aspect, the present disclosure provides a composition comprising a compound of formula (I):

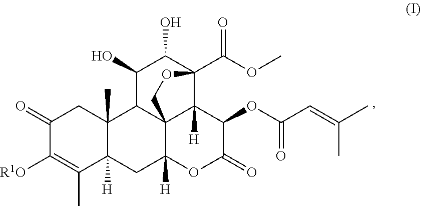

wherein:

R¹ is selected from the group consisting of

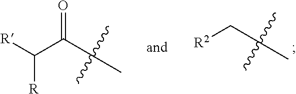

R and R′ are each independently selected from the group consisting of H, optionally substituted C₁-C₇ alkyl, optionally substituted C₁-C₆ aminoalkyl, optionally substituted C₃-C₇ cycloalkyl, optionally substituted C₆-C₁₀ aryl, optionally substituted benzyl, N(R^{a})(R^{b}), SR^{a}, OR^{a}, and C(═O)OR^{a},
or R and R′ combine with the atom to which they are bound to form

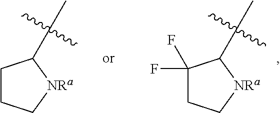

wherein each optional substituent in R and R′ is at least one selected from the group consisting of N(R^{a})(R^{b}), benzyl, C₁-C₆ alkyl, C₃-C₇ cycloalkyl, C₁-C₆ heterocycloalkyl, C(═O)C₁-C₃ alkyl, C(═O)C₃-C₇ cycloalkyl, C(═O)(C₁-C₃ alkyl)N(R^{a})(R^{b}), C₁-C₃ haloalkyl, halogen, C(═O)OR^{a}, OR^{a}, SR^{a}, imidazolyl, and N(R^{a})C(═NR^{e})N(R^{e})(R^{b});

R² is selected from the group consisting of C(═O)N(R^{e})(R^{c}), C(═O)OR^{a}, and C(═O)R^{a};

each occurrence of R^{a} and R^{b} is independently selected from the group consisting of H, C₁-C₆ alkyl, C₃-C₇ cycloalkyl, C(═O)C₁-C₆ alkyl, C(═O)O(C₁-C₆ alkyl), C(═O)(C₁-C₃ alkyl)NH₂, C(═O)(C₁-C₃ alkyl)NHC(═O)(C₁-C₃ alkyl)NH₂, and S(═O)₂(C₁-C₃ alkyl);

each occurrence of R^{c} is independently selected from the group consisting of H, C₁-C₃ alkyl-N(R^{a})(R^{b}), C₁-C₃ alkyl-C(═O)OR^{a}, and CH(CO₂R^{a})CH₂Ph; and

each occurrence of R^{e} is independently selected from the group consisting of H, C₁-C₆ alkyl, and C₃-C₇ cycloalkyl;

or a pharmaceutically acceptable salt, solvate, or polymorph thereof.

In certain embodiments, R is C₁-C₃ alkyl optionally substituted with NH₂ or NHBoc.

In certain embodiments, the compound is

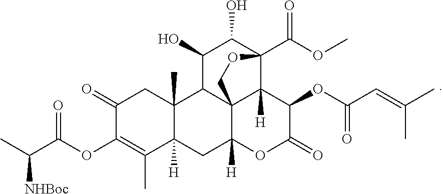

In certain embodiments, the compound is

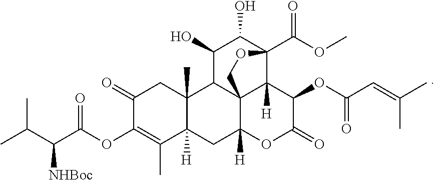

In certain embodiments, the compound is

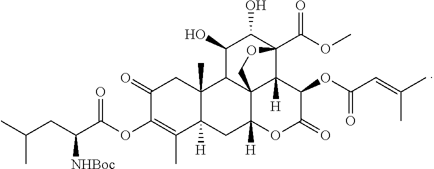

In certain embodiments, the compound is

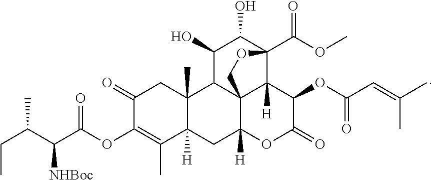

In certain embodiments, the compound is

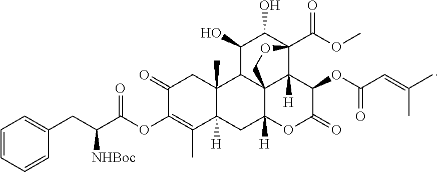

In certain embodiments, the compound is

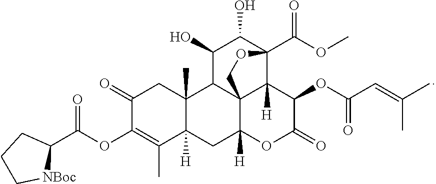

In certain embodiments, the compound is

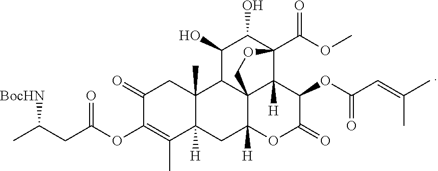

In certain embodiments, the compound is

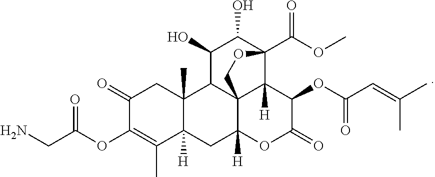

In certain embodiments, the compound is

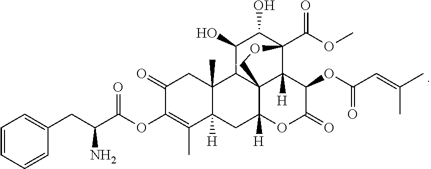

In certain embodiments, the compound is

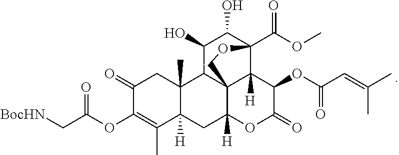

In certain embodiments, the compound is

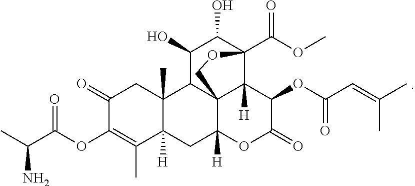

In certain embodiments, the compound is

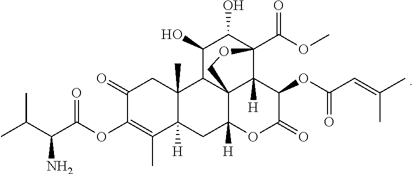

In certain embodiments, the compound is

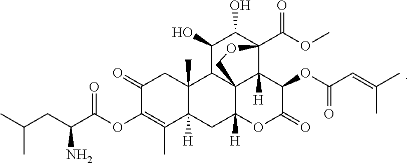

In certain embodiments, the compound is

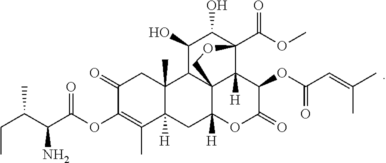

In certain embodiments, the compound is

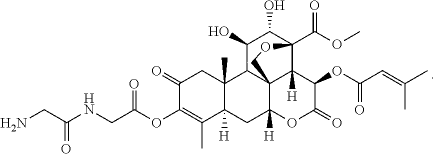

In certain embodiments, the compound is

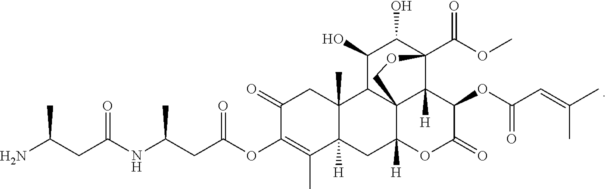

In certain embodiments, the compound is

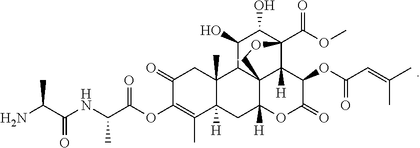

In certain embodiments, the compound is

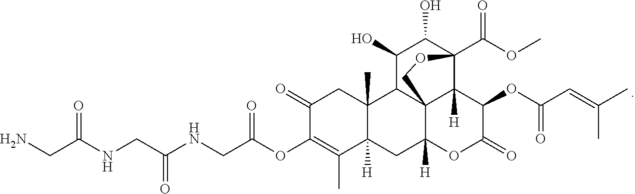

In certain embodiments, the compound is

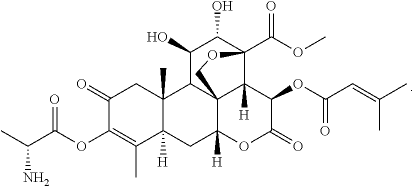

In certain embodiments, the compound is

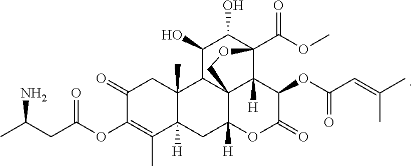

In certain embodiments, the compound is

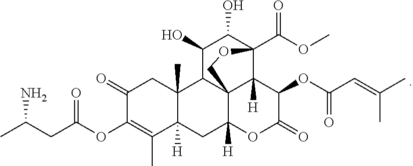

In certain embodiments, the compound is

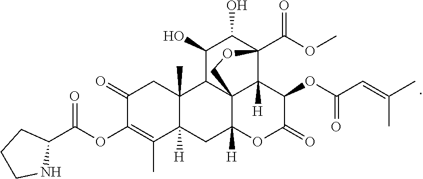

In certain embodiments, the compound is

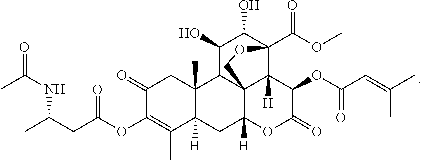

In certain embodiments, the compound is

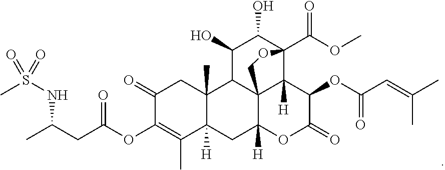

In certain embodiments, the compound is

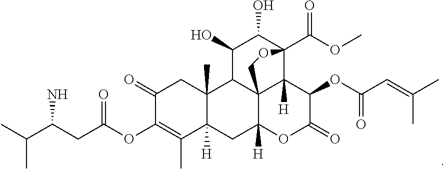

In certain embodiments, the compound is

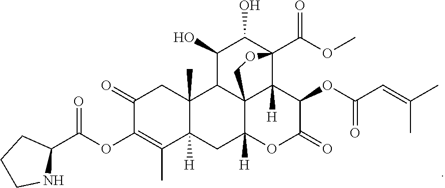

In certain embodiments, the compound is

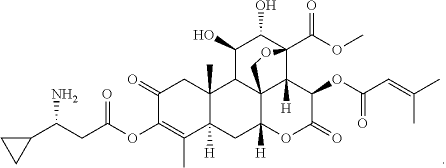

In certain embodiments, the compound is

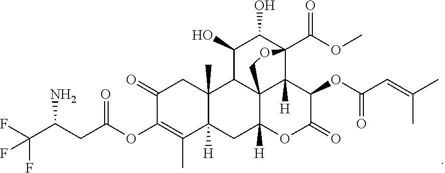

In certain embodiments, the compound is

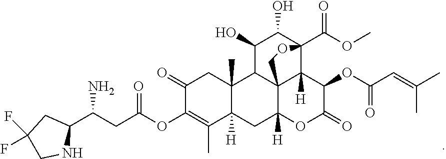

In certain embodiments, the compound is

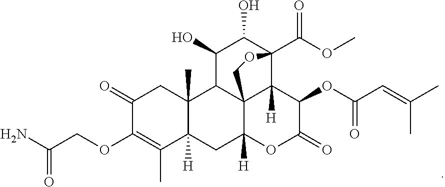

In certain embodiments, the compound is

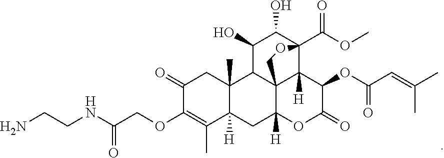

In certain embodiments, the compound is

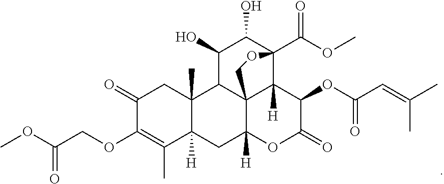

In certain embodiments, the compound is

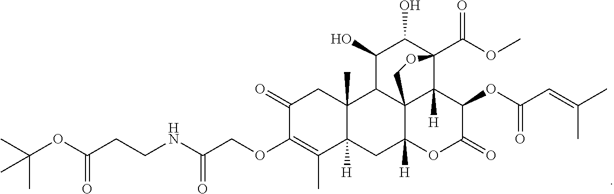

In certain embodiments, the compound is

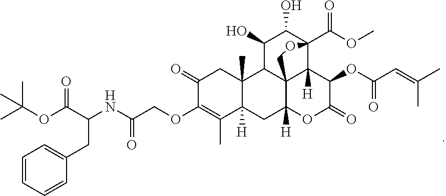

In certain embodiments, the compound is

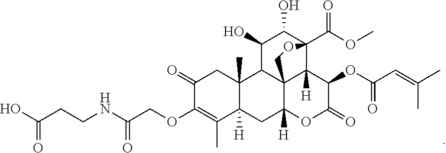

In certain embodiments, the compound is

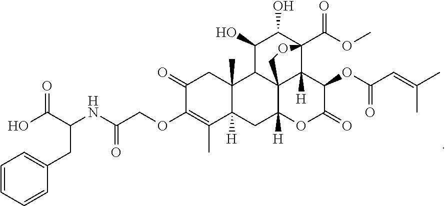

In certain embodiments, the compound is

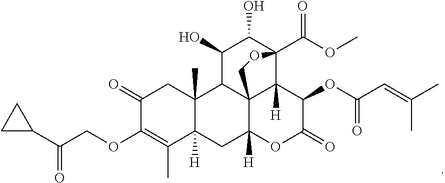

In certain embodiments, the compound is

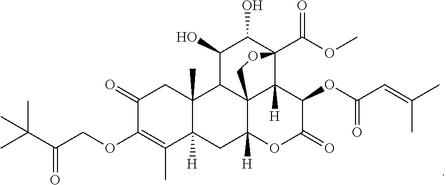

In certain embodiments, the compound is

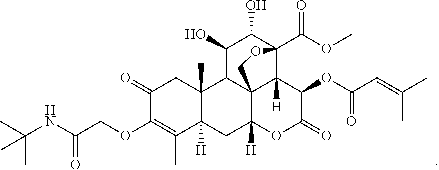

In certain embodiments, the compound is

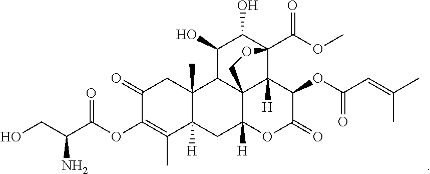

In certain embodiments, the compound is

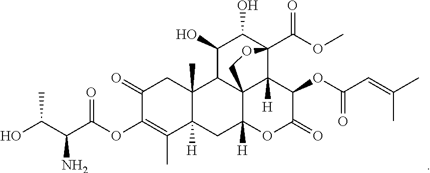

In certain embodiments, the compound is

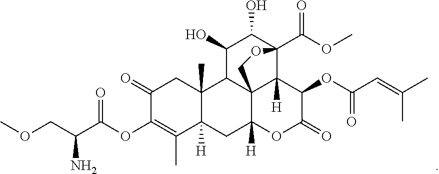

In certain embodiments, the compound is

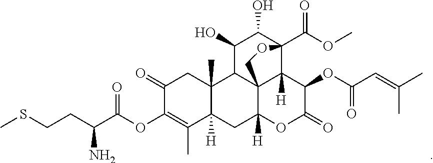

In certain embodiments, the compound is

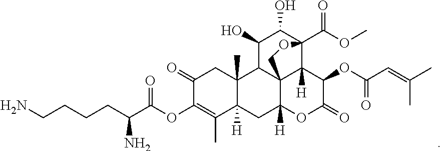

In certain embodiments, the compound is

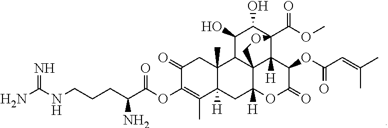

In certain embodiments, the compound is

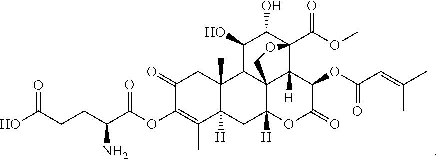

In certain embodiments, the compound is

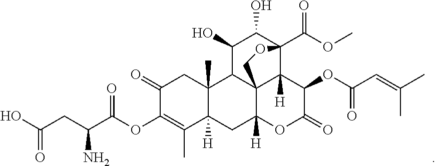

In certain embodiments, the compound is

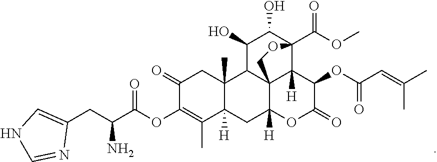

In certain embodiments, the compound is

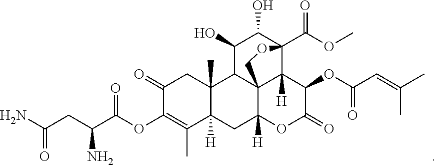

In certain embodiments, the compound is

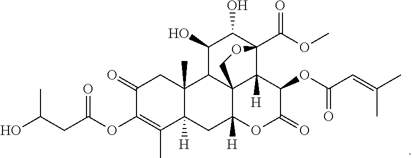

In another aspect, the present disclosure provides a composition comprising a compound of formula (Ib):

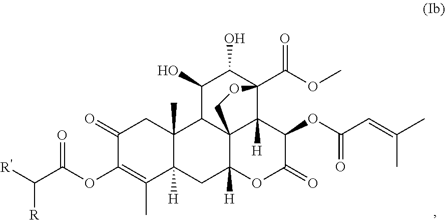

wherein R and R′ are each independently selected from the group consisting of optionally substituted C₁-C₆ alkyl, benzyl, NH₂, NHBoc and H,

or R and R′ combine with the atom to which they are bound to form

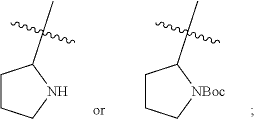 [SMILES: *C1CCCN1 ; *C1CCCN1C(=O)OC(C)(C)C]

or a pharmaceutically acceptable salt, solvate or polymorph thereof.

In certain embodiments, R is C₁-C₃ alkyl optionally substituted with NH₂ or NHBoc.

In certain embodiments, the compound is

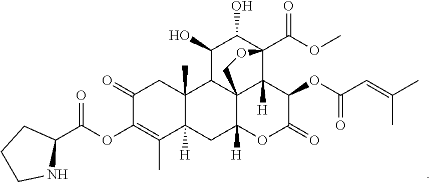

In certain embodiments, the compound is

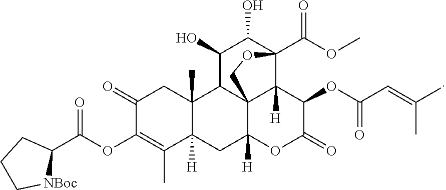

In certain embodiments, the compound is

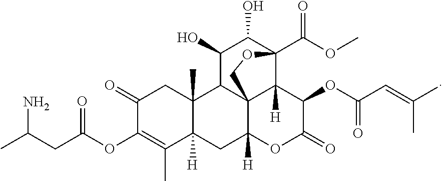

In certain embodiments, the compound is

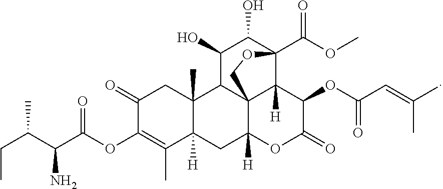

In certain embodiments, the compound is

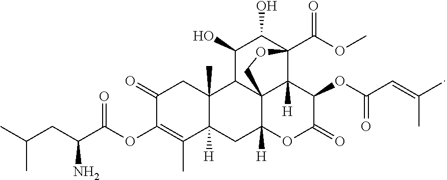

In certain embodiments, the compound is

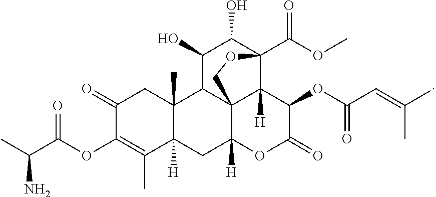

In certain embodiments, the compound is

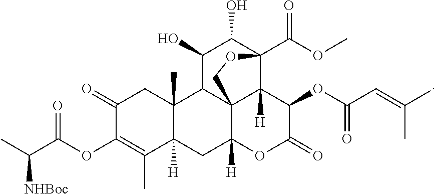

In certain embodiments, the compound is

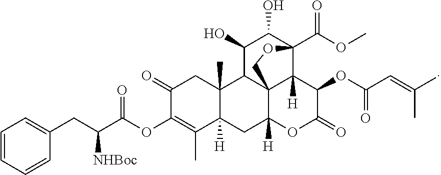

In certain embodiments, the compound is

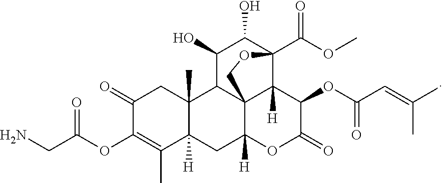

In certain embodiments, the compound is

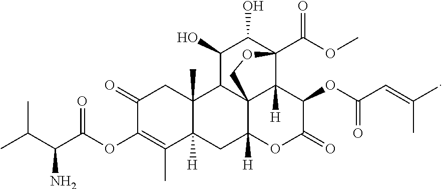

In certain embodiments, the compound is

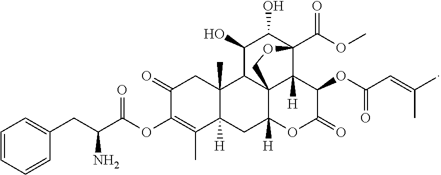

In certain embodiments, the compound is

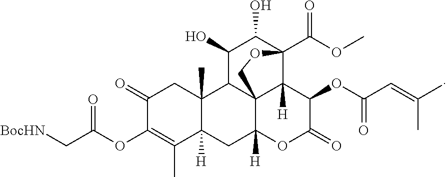

In another aspect, the present disclosure provides a composition comprising a compound of formula (IIa):

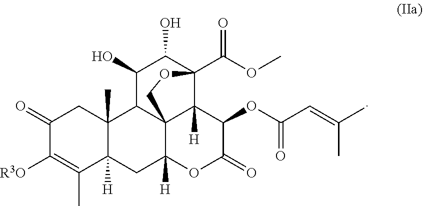

wherein:

R³ is selected from the group consisting of

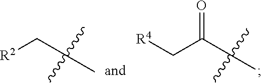

R² is selected from the group consisting of C(═O)N(R^{e})(R^{c}), C(═O)OR^{a}, and C(═O)R^{a};

R⁴ is selected from the group consisting of C₁-C₇ alkyl, C₁-C₇ cycloalkyl, C₁-C₃ aminoalkyl, C₆-C₁₀ aryl, benzyl, OR^{a}, N(R^{a})(R^{b}), SR^{a}, and C(═O)OR^{a},
wherein the C₁-C₇ alkyl, C₁-C₇ alkyl, C₁-C₃ aminoalkyl, C₆-C₁₀ aryl, or benzyl in R⁴ is substituted with at least one substituent selected from the group consisting of OR^{a}, SR^{a}, N(R^{a})(R^{d}), S(═O)₂(C₁-C₃ alkyl), C₃-C₇ cycloalkyl, C₁-C₆ heterocycloalkyl, and halogen;

each occurrence of R^{a} and R^{b} is independently selected from the group consisting of H, C₁-C₆ alkyl, C₃-C₇ cycloalkyl, C(═O)O(C₁-C₆ alkyl), C(═O)(C₁-C₃ alkyl)NH₂, and S(═O)₂(C₁-C₃ alkyl);

each occurrence of R^{c} is independently selected from the group consisting of H, C₁-C₃ alkyl-N(R^{a})(R^{b}), C₁-C₃ alkyl-C(═O)OR^{a}, and CH(CO₂R^{a})CH₂Ph;

each occurrence of R^{d} is independently selected from the group consisting of C₁-C₆ alkyl, C₃-C₇ cycloalkyl, C(═O)C₁-C₆ alkyl, C(═O)O(C₁-C₆ alkyl), C(═O)(C₁-C₃ alkyl)NH₂, and S(═O)₂(C₁-C₃);

or a pharmaceutically acceptable salt, solvate, or polymorph thereof.

In certain embodiments, the compound is

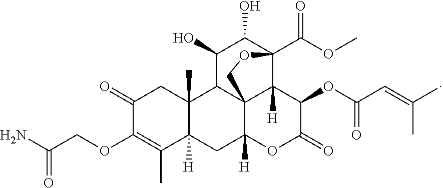

In certain embodiments, the compound is

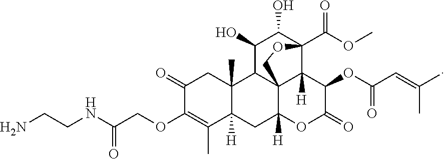

In certain embodiments, the compound is

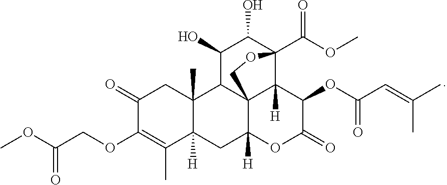

In certain embodiments, the compound is

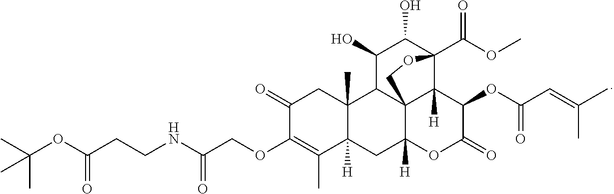

In certain embodiments, the compound is

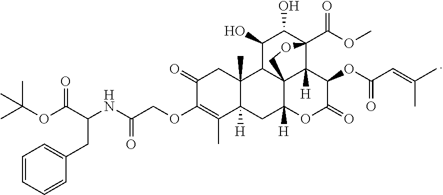

In certain embodiments, the compound is

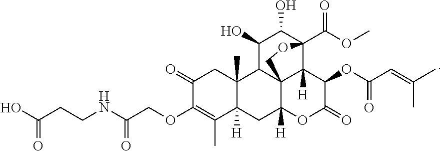

In certain embodiments, the compound is

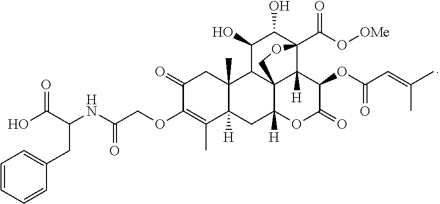

In certain embodiments, the compound is

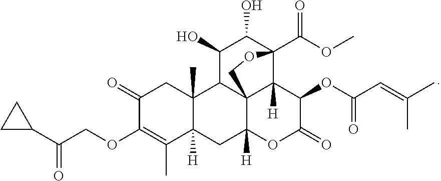

In certain embodiments, the compound is

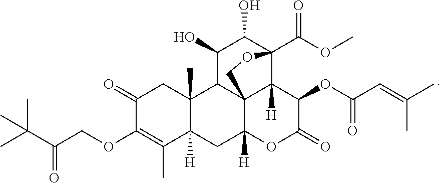

In certain embodiments, the compound is

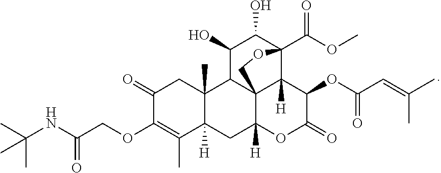

In certain embodiments, the compound is

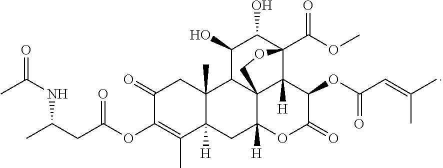

In certain embodiments, the compound is

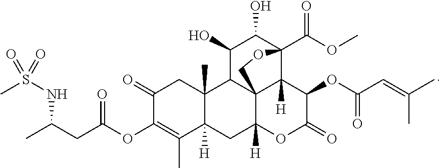

In certain embodiments, the compound is

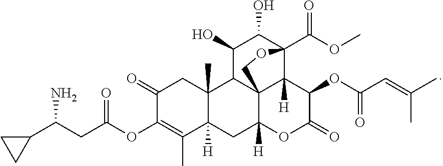

In certain embodiments, the compound is

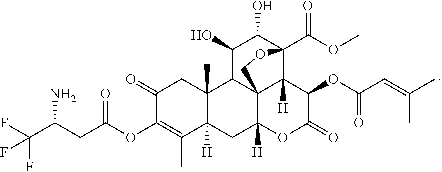

In certain embodiments, the compound is

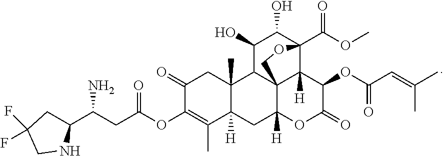

In certain embodiments, the compound is

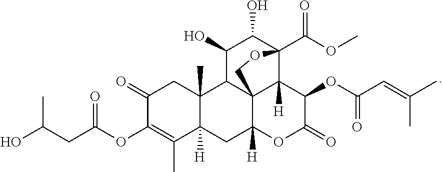

In another aspect, the present disclosure provides a composition comprising a compound of formula (IIb):

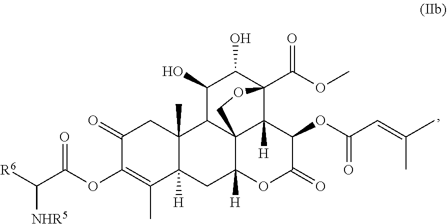

wherein:

R⁵ is selected from the group consisting of H, C₁-C₆ alkyl, and C(═O)O(C₁-C₆ alkyl);

R⁶ is selected from the group consisting of C₁-C₇ alkyl, C₁-C₇ cycloalkyl, C₆-C₁₀ aryl, benzyl, OR^{a}, N(R^{a})(R^{b}), SR^{a}, and C(═O)OR^{a},
wherein the C₁-C₇ alkyl, C₁-C₇ alkyl, C₆-C₁₀ aryl, or benzyl in R⁵ is substituted with at least one substituent selected from the group consisting of C₃-C₇ cycloalkyl, C₁-C₆ heterocycloalkyl, OR^{a}, SR^{a}, N(R^{a})(R^{b}), NR^{a}C(═NR^{a})N(R^{a})(R^{b}), C(═O)OR^{a}, C(═O)N(R^{a})(R^{b}), halogen, and imidazolyl; and

each occurrence of R^{a} and R^{b} is independently selected from the group consisting of H, C₁-C₆ alkyl, C₃-C₇ cycloalkyl, C(═O)C₁-C₆ alkyl, C(═O)O(C₁-C₆ alkyl), C(═O)(C₁-C₃ alkyl)NH₂, and S(═O)₂(C₁-C₃ alkyl);

or a pharmaceutically acceptable salt, solvate, or polymorph thereof.

In certain embodiments, the compound is

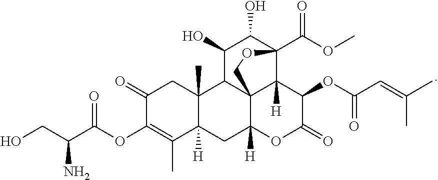

In certain embodiments, the compound is

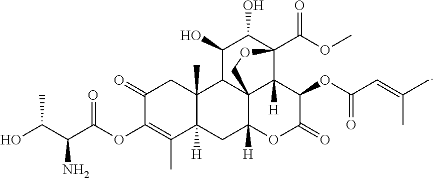

In certain embodiments, the compound is

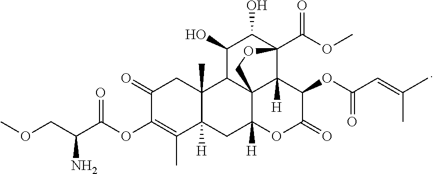

In certain embodiments, the compound is

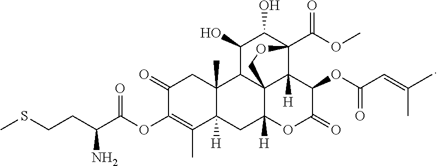

In certain embodiments, the compound is

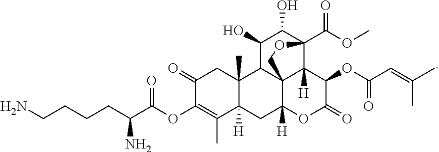

In certain embodiments, the compound is

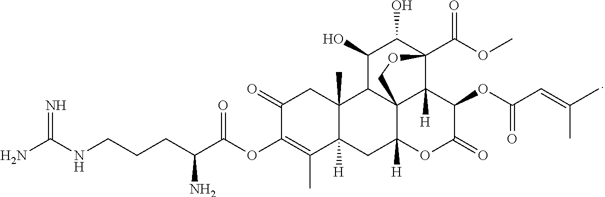

In certain embodiments, the compound is

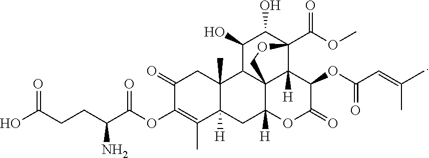

In certain embodiments, the compound is

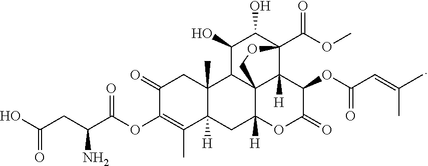

In certain embodiments, the compound is

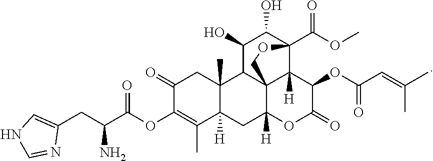

In certain embodiments, the compound is

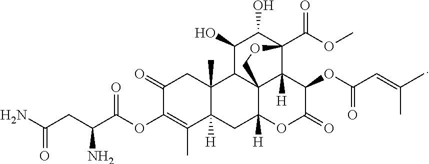

In another aspect, the present disclosure provides a composition comprising any one of the compounds of the present disclosure, further comprising at least one pharmaceutically acceptable carrier.

In various embodiments, R^{a} is H. In various embodiments, R^{a} is Me. In various embodiments, R^{a} is t-Bu. In various embodiments, R^{a} is C(═O)Ot-Bu. In various embodiments, R^{a} is C(═O)CH₂NH₂. In various embodiments, R^{a} is S(═O)₂Me. In various embodiments, R^{a} is C(═O)Me.

In various embodiments, R^{b} is H. In various embodiments, R^{b} is Me. In various embodiments, R^{b} is t-Bu. In various embodiments, R^{b} is C(═O)Ot-Bu. In various embodiments, R^{b} is C(═O)CH₂NH₂. In various embodiments, R^{b} is S(═O)₂Me. In various embodiments, R^{b} is C(═O)Me.

In various embodiments, R^{c} is CH₂CH₂NH₂. In various embodiments, R^{c} is CH₂CH₂C(═O)Ot-Bu. In various embodiments, R^{c} is CH₂CH₂C(═O)OH. In various embodiments, R^{c} is CH(C(═O)Ot-Bu)benzyl. In various embodiments, R^{c} is CH(C(═O)OH)benzyl.

In various embodiments, R^{d} is S(═O)₂Me. In various embodiments, R^{d} is C(═O)Me.

In various embodiments, R^{e} is H. In various embodiments, R^{e} is Me.

In certain embodiments, the compound of formula I is a compound of formula Ib. In certain embodiments, the compound of formula I is a compound of formula IIa. In certain embodiments, the compound of formula I is a compound of formula IIb.

In various embodiments, the composition further comprises at least one pharmaceutically acceptable carrier. The composition may be formulated for administration by any route known in the art. Pharmaceutical carriers suitable for use in combination with various embodiments of the invention are described under Administration.

### Methods

In another aspect, the invention further provides a method of treating a hematologic malignancy in a subject in need thereof, the method comprising administering to the subject a therapeutically effective amount of a composition of the invention. In various embodiments, the hematologic malignancy is selected from the group consisting of leukemia and lymphoma. In various embodiments, the hematologic malignancy is acute myeloid leukemia, Burkitt's lymphoma, B-cell lymphoma, hepatocellular carcinoma, acute promyelocytic leukemia, plasmacytoma, myeloma, chronic myelogenous leukemia, Epstein-Barr Virus (EBV) associated lymphoma, acute lymphoblastic leukemia, acute promyelocytic leukemia, large cell lymphoma, mantle cell lymphoma, non-Hodgkin's B-cell lymphoma. In various embodiments, the hematologic malignancy is Epstein-Barr Virus (EBV) associated lymphoma. In various embodiments, the subject is a mammal. In various embodiments, the subject is a human.

### Synthesis

The present disclosure further provides methods of preparing the compounds of the present disclosure. Compounds of the present teachings can be prepared in accordance with the procedures outlined herein, from commercially available starting materials, compounds known in the literature, or readily prepared intermediates, by employing standard synthetic methods and procedures known to those skilled in the art. Standard synthetic methods and procedures for the preparation of organic molecules and functional group transformations and manipulations can be readily obtained from the relevant scientific literature or from standard textbooks in the field.

It is appreciated that where typical or preferred process conditions (i.e., reaction temperatures, times, mole ratios of reactants, solvents, pressures, and so forth) are given, other process conditions can also be used unless otherwise stated. Optimum reaction conditions can vary with the particular reactants or solvent used, but such conditions can be determined by one skilled in the art by routine optimization procedures. Those skilled in the art of organic synthesis will recognize that the nature and order of the synthetic steps presented can be varied for the purpose of optimizing the formation of the compounds described herein.

The processes described herein can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry, or by chromatography such as high pressure liquid chromatography (HPLC), gas chromatography (GC), gel-permeation chromatography (GPC), or thin layer chromatography (TLC).

Preparation of the compounds can involve protection and deprotection of various chemical groups. The need for protection and deprotection and the selection of appropriate protecting groups can be readily determined by one skilled in the art. The chemistry of protecting groups can be found, for example, in Greene, et al., Protective Groups in Organic Synthesis, 2d. Ed. (Wiley & Sons, 1991), the entire disclosure of which is incorporated by reference herein for all purposes.

The reactions or the processes described herein can be carried out in suitable solvents that can be readily selected by one skilled in the art of organic synthesis. Suitable solvents typically are substantially nonreactive with the reactants, intermediates, and/or products at the temperatures at which the reactions are carried out, i.e., temperatures that can range from the solvent's freezing temperature to the solvent's boiling temperature. A given reaction can be carried out in one solvent or a mixture of more than one solvent. Depending on the particular reaction step, suitable solvents for a particular reaction step can be selected.

A compound of formula (I) can be prepared, for example, according to the synthetic methods outlined in Schemes 1-2, wherein the substituents are defined within the scope of the present disclosure.

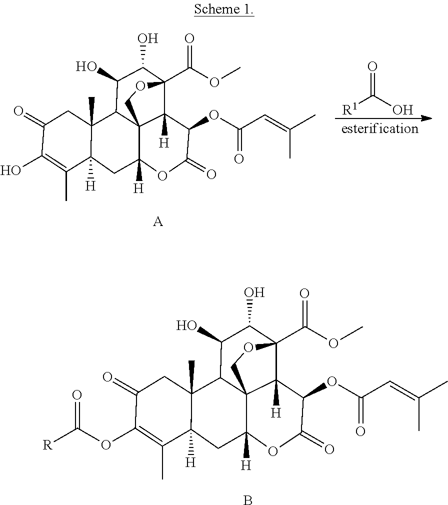

In certain embodiments, the compound of formula I is a compound of formula B. In certain embodiments, the compound of formula I is prepared by esterification. In certain embodiments, the esterification comprises treatment of A with EDC.HCl and DMAP in the presence of R¹—C(═O)OH. In certain embodiments, R¹ comprises a Boc-protected amine. In certain embodiments, the Boc-protected amine is deprotected with a suitable acid. In certain embodiments, a suitable acid comprises aqueous HCl, TFA, HCl in Et₂O, and/or HCl in dioxane.

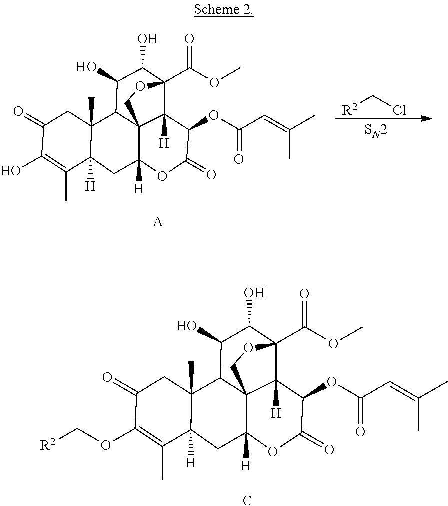

In certain embodiments, the compound of formula I is a compound of formula C. In certain embodiments, the compound of formula I is prepared by an S_{N}2 reaction. In certain embodiments, the S_{N}2 reaction comprises treatment of C with a suitable base in the presence of a primary alkyl halide (e.g. R²CH₂Cl). In certain embodiments, a suitable base comprises K₂CO₃ or Et₃N. In certain embodiments, R² comprises a Boc-protected amine. In certain embodiments, the Boc-protected amine is deprotected with a suitable acid. In certain embodiments, a suitable acid comprises aqueous HCl, TFA, HCl in Et₂O, and/or HCl in dioxane.

### Administration/Dosage/Formulations

The regimen of administration may affect what constitutes an effective amount. The therapeutic formulations may be administered to the subject either prior to or after the onset of a disease or disorder contemplated in the invention. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a patient, preferably a mammal, more preferably a human, may be carried out using known procedures, at dosages and for periods of time effective to treat a disease or disorder contemplated in the invention. An effective amount of the therapeutic compound necessary to achieve a therapeutic effect may vary according to factors such as the state of the disease or disorder in the patient; the age, sex, and weight of the patient; and the ability of the therapeutic compound to treat a disease or disorder contemplated in the invention. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the invention is from about 1 and 5,000 mg/kg of body weight/per day. The pharmaceutical compositions useful for practicing the invention may be administered to deliver a dose of from ng/kg/day and 100 mg/kg/day. In certain embodiments, the invention envisions administration of a dose which results in a concentration of the compound of the present invention from 1 μM and 10 μM in a mammal. One of ordinary skill in the art would be able to study the relevant factors and make the determination regarding the effective amount of the therapeutic compound without undue experimentation.

Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient.

In particular, the selected dosage level depends upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for the treatment of a disease or disorder contemplated in the invention.

In certain embodiments, the compositions of the invention are formulated using one or more pharmaceutically acceptable excipients or carriers. In other embodiments, the pharmaceutical compositions of the invention comprise a therapeutically effective amount of a compound of the invention and a pharmaceutically acceptable carrier.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin.

In certain embodiments, the compositions of the invention are administered to the patient in dosages that range from one to five times per day or more. In other embodiments, the compositions of the invention are administered to the patient in range of dosages that include, but are not limited to, once every day, every two, days, every three days to once a week, and once every two weeks. It is readily apparent to one skilled in the art that the frequency of administration of the various combination compositions of the invention varies from individual to individual depending on many factors including, but not limited to, age, disease or disorder to be treated, gender, overall health, and other factors. Thus, the invention should not be construed to be limited to any particular dosage regime and the precise dosage and composition to be administered to any patient is determined by the attending physical taking all other factors about the patient into account.

Compounds of the invention for administration may be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 3050 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments therebetween.

In some embodiments, the dose of a compound of the invention is from about 1 mg and about 2,500 mg. In some embodiments, a dose of a compound of the invention used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, the present invention is directed to a packaged pharmaceutical composition comprising a container holding a therapeutically effective amount of a compound of the invention, alone or in combination with a second pharmaceutical agent; and instructions for using the compound to treat, prevent, or reduce one or more symptoms of a disease or disorder contemplated in the invention.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents.

Routes of administration of any of the compositions of the invention include oral, nasal, rectal, intravaginal, parenteral, buccal, sublingual or topical. The compounds for use in the invention may be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

### Oral Administration

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

In certain embodiments, the tablets of the invention comprise saracatinib difumarate, mannitol, dibasic calcium phosphate anhydrous, crospovidone, hypromellose and magnesium stearate, with a film-coat containing hypromellose, macrogol 400, red iron oxide, black iron oxide and titanium dioxide. In other embodiments, the tablets of the invention comprise about 50 or 125 mg of saracatinib expressed as free base. In yet other embodiments, the tablets of the invention comprise about 71.4 or 178.6 mg of saracatinib expressed as difumarate salt.

For oral administration, the compounds of the invention may be in the form of tablets or capsules prepared by conventional means with pharmaceutically acceptable excipients such as binding agents (e.g., polyvinylpyrrolidone, hydroxypropylcellulose or hydroxypropylmethylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulfate). If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

Granulating techniques are well known in the pharmaceutical art for modifying starting powders or other particulate materials of an active ingredient. The powders are typically mixed with a binder material into larger permanent free-flowing agglomerates or granules referred to as a “granulation”. For example, solvent-using “wet” granulation processes are generally characterized in that the powders are combined with a binder material and moistened with water or an organic solvent under conditions resulting in the formation of a wet granulated mass from which the solvent must then be evaporated.

Melt granulation generally consists in the use of materials that are solid or semi-solid at room temperature (i.e. having a relatively low softening or melting point range) to promote granulation of powdered or other materials, essentially in the absence of added water or other liquid solvents. The low melting solids, when heated to a temperature in the melting point range, liquefy to act as a binder or granulating medium. The liquefied solid spreads itself over the surface of powdered materials with which it is contacted, and on cooling, forms a solid granulated mass in which the initial materials are bound together. The resulting melt granulation may then be provided to a tablet press or be encapsulated for preparing the oral dosage form. Melt granulation improves the dissolution rate and bioavailability of an active (i.e. drug) by forming a solid dispersion or solid solution.

U.S. Pat. No. 5,169,645 discloses directly compressible wax-containing granules having improved flow properties. The granules are obtained when waxes are admixed in the melt with certain flow improving additives, followed by cooling and granulation of the admixture. In certain embodiments, only the wax itself melts in the melt combination of the wax(es) and additives(s), and in other cases both the wax(es) and the additives(s) melt.

The present invention also includes a multi-layer tablet comprising a layer providing for the delayed release of one or more compounds of the invention, and a further layer providing for the immediate release of a medication for treatment of a disease or disorder contemplated in the invention. Using a wax/pH-sensitive polymer mix, a gastric insoluble composition may be obtained in which the active ingredient is entrapped, ensuring its delayed release.

### Parenteral Administration

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intravenous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multidose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In certain embodiments of a formulation for parenteral administration, the active ingredient is provided in dry (i.e., powder or granular) form for reconstitution with a suitable vehicle (e.g., sterile pyrogen free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butanediol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

### Additional Administration Forms

Additional dosage forms of this invention include dosage forms as described in U.S. Pat. Nos. 6,340,475; 6,488,962; 6,451,808; 5,972,389; 5,582,837; and 5,007,790. Additional dosage forms of this invention also include dosage forms as described in U.S. Patent Applications Nos. 20030147952; 20030104062; 20030104053; 20030044466; 20030039688; and 20020051820. Additional dosage forms of this invention also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

### Controlled Release Formulations and Drug Delivery Systems

In certain embodiments, the formulations of the present invention may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material that provides sustained release properties to the compounds. As such, the compounds for use the method of the invention may be administered in the form of microparticles, for example, by injection or in the form of wafers or discs by implantation.

In certain embodiments, the compounds of the invention are administered to a patient, alone or in combination with another pharmaceutical agent, using a sustained release formulation.

The term delayed release is used herein in its conventional sense to refer to a drug formulation that provides for an initial release of the drug after some delay following drug administration and that may, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

### Dosing

The therapeutically effective amount or dose of a compound of the present invention depends on the age, sex and weight of the patient, the current medical condition of the patient and the progression of a disease or disorder contemplated in the invention. The skilled artisan is able to determine appropriate dosages depending on these and other factors.

A suitable dose of a compound of the present invention may be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

In the case wherein the patient's status does improve, upon the doctor's discretion the administration of the inhibitor of the invention is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced, as a function of the disease or disorder, to a level at which the improved disease is retained. In certain embodiments, patients require intermittent treatment on a long-term basis upon any recurrence of symptoms and/or infection.

The compounds for use in the method of the invention may be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Toxicity and therapeutic efficacy of such therapeutic regimens are optionally determined in cell cultures or experimental animals, including, but not limited to, the determination of the LD50 (the dose lethal to 50% of the population) and the ED50 (the dose therapeutically effective in 50% of the population). The dose ratio between the toxic and therapeutic effects is the therapeutic index, which is expressed as the ratio between LD50 and ED50. The data obtained from cell culture assays and animal studies are optionally used in formulating a range of dosage for use in human. The dosage of such compounds lies preferably within a range of circulating concentrations that include the ED50 with minimal toxicity. The dosage optionally varies within this range depending upon the dosage form employed and the route of administration utilized.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The following examples further illustrate aspects of the present invention. However, they are in no way a limitation of the teachings or disclosure of the present invention as set forth herein.

### Experimental Examples

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

The Materials and Methods used in the following experiments are here described.

### Cell Lines, Antibodies, Primers, and Compounds

Cell lines, antibodies, primers, and compounds used in this study are summarized in Table 5.

### General Methods for Chemistry

All reactions were carried out under argon atmosphere. Preparative high-performance liquid chromatography (HPLC) was performed using a Gilson 331 and 332 pumps with a UV/VIS-155 detector and GX-271 liquid handler. Column was Phenomenex Luna LC Column (5 μm C18 100 Å, 150×21.2 mm). ¹H NMR spectra were recorded on a 300 MHz INOVA VARIAN spectrometer. Chemical shift values are given in ppm and referred as the internal standard to TMS (tetramethylsilane). The coupling constants (J) are reported in Hertz (Hz). Mass Spectra were obtained on an Agilent 6120 mass spectrometer with electrospray ionization source (1200 Aligent LC-MS spectrometer, Positive). Mobile phase flow was 1.0 mL/min with a 3.0 min gradient from 20% aqueous media (0.1% formic acid) to 95% CH₃CN (0.1% formic acid) and a 9.0 min total acquisition time. All the tested compounds possess a purity of at least 95%, which was determined by LC/MS Data recorded using an Agilent 1200 liquid chromatography and Agilent 6120 mass spectrometer, and further supported by clean NMR spectra.

### Cell Viability Assay

5000 cells as indicated were cultured in 96-well plate with 100 μl of media. On the next day, Brusatol or associated analogs were added once to the cells without media change. After treatment, the cells were incubated with 100 μl buffer for 10 minutes at room temperature following the instructions of CellTiter-Glo kit (Promega, Madison, Wis.). Luminescence was detected using a multi-mode reader Cytation 5 (BioTek, Winooski, Vt.). Brusatol or its analogs are dissolved in 100% ethanol and diluted in 1×PBS for treatment. Two independent experiments done in duplicates were performed.

### Cell Cycle Assay

5 million cells were harvested, fixed with 80% ethanol for 2 hours or overnight at −20° C., then washed with PBS and incubated with Propidium iodide (PI) staining buffer (0.5 mg/ml propidium iodide in PBS with 50 μg/ml RNase A) for 30 minutes at room temperature. The stained cells were resuspended in PBS and analyzed on a FACSCalibur system (Becton Dickinson, San Jose, Calif., USA) using FlowJo software (TreeStar, San Carlos, Calif., USA).

### Mass Spectrometry (MS)

200 million Raji cells were harvested and lysed with RIPA buffer [1% Nonidet P-40 (NP-40), 10 mM Tris (pH8.0), 2 mM EDTA, 150 mM NaCl, supplemented with protease inhibitors (1 mM phenylmethylsulphonyl fluoride (PMSF), 1 μg/ml each Aprotinin, Pepstatin, and Leupeptin)]. 20 mg lysates were combined with magnetic Streptavidin beads as well as biotin-conjugated compounds 51046, 51048, or 51052, respectively. The complexes were incubated overnight with rotation at 4° C. The immunoprecipitated samples were washed with RIPA three times and resolved on an SDS-PAGE gel.

Briefly, the peptides were extracted from the gel band and analyzed with an Orbitrap Fusion (Thermo Fisher Scientific, San Jose, Calif., USA) attached to an EasyLC 1000 system (Thermo Fisher Scientific, San Jose, Calif., USA) at 400 nL/min. The raw MS data were processed using MaxQuant (Version 1.5.3.30), and searched with the UniProt human database. Then the target-decoy approach was used to filter the search results, in which the false discovery rate was less than 1% at the peptide and protein level⁶².

### RNA-Seq and Data Analysis

LCL1 cells were untreated or treated with Brusatol for 72 hours, then total RNAs from these cells were extracted with Trizol (Invitrogen, Inc., Carlsbad, Calif.) as previously described. The cDNA library was prepared with the commercial Illumina library preparation kits (TruSeq Stranded RNA LT Ribo-Zero H/M/R Kit) according to the manufacturer's protocols and sequenced with an Illumina HiSeq2000 instrument. Quality check was performed on the raw RNA-Seq reads (FastQC), and the adapters were cut (Trim Galore) and mapped with hg38 genome (HISAT2). Differentially expressed transcripts were defined with Cuffdiff tools⁶⁵ and visualized using R software. Signaling pathway analysis was conducted with Ingenuity Pathway Analysis program (QIAGEN Inc.).

### Real-Time Quantitative PCR

Real-time PCR experiments were performed as previously described. The experiments were performed in triplicate.

### Proteins Expression and Purification

PIK3CG cDNA (Plasmid #20574) was purchased from Addgene and cloned into a pGEX-6P-1 vector (GE Healthcare, Madison, Wis., USA). This GST-tagged PIK3CG plasmid was transformed into E. coli BL21 (DE3)-competent cells (Life Technologies, Carlsbad, Calif., USA) for in vitro expression according to Molecular Cloning (Third Edition).

### Pull-Down Assay

30 μg of purified GST-tagged proteins were pre-cleared with Dynabeads M-280 Streptavidin (Invitrogen, Carlsbad, Calif., USA). The supernatants were collected and incubated with Dynabeads M-280 Streptavidin and 0, 300 μM, or 500 μM of biotin-conjugated Brusatol derivatives (51052) overnight at 4° C. For the competitive binding assays, cell lysates from SU-DHL-4 cells were incubated with the biotin-conjugated Brusatol derivative (51048) alone or together with Brusatol as well as Dynabeads M-280 Streptavidin. The complexes were harvested and washed with PBS. The collected beads were mixed with SDS loading buffer for further analysis.

### Western Blot

Western blot analyses were performed as previously described. Briefly, total cell lysates were separated by SDS-PAGE gel and transferred to nitrocellulose membrane. The membranes were blocked with 5% non-fat milk, probed with specific antibodies, and visualized using the Odyssey scanner (LiCor Inc., Lincoln, Nebr.).

### Generation of CRISPR Knockout Cell Lines

Oligos were obtained from Integrated DNA Technologies (IDT; Coralville, Iowa, USA) and cloned into the lentiviral vector lentiCRISPR v2 (Addgene #52961). Lentivirus production and transduction have been described previously. Here, Raji cells were transduced with a lentivirus harboring lentiCRISPR v2 vector or a construct containing the guide sequence (SEQ ID NO: 1 GAACGGAGAAGAGATTCACG) against PIK3CG gene. At 48 hours post-transduction, cells were selected with puromycin and harvested to determine PIK3CG expression with western blot.

### Surveyor Mutation Detection Assay

Genomic DNAs were extracted with DNeasy Blood & Tissue Kits (Qiagen, Valencia, Calif., USA). PCR amplicons of target regions were analyzed for the mismatch mutations using Surveyor Mutation Detection Kit (Integrated DNA Technologies) according to the manufacturer's instructions.

### In Vivo Tumorigenic Assays Using Xenografts

Six-week-old male NOD.CB17-Prkdc^{scid}/J (NOD/SCID) mice (Jackson Labs, Bar Harbor, Me., USA) were used as the human-in-mouse xeno-transplantation model. MOLM14 cells (1×10⁷ cells) were injected into the subcutaneous space on the left flank of the mice. Once xenografts reached a size of 100 mm³, Brusatol and analogs (14, 15, 26, 31) at 2 mg/kg body weight or PBS were injected intraperitoneally three times per week. Body weight and tumor size were monitored before every injection. After treatment for two weeks, the mice were euthanized by CO₂ inhalation and the tumors were carefully excised. The tumor volume (mm³) was measured and calculated using the formula (a×b×b)/2 (a, the largest diameter of two measurements; b, the smallest diameter of two measurements).

### In Vivo Toxicity Test

To determine the toxicity of Brusatol, 15 and 26 compounds, six-week-old male NOD.CB17-Prkdc^{scid}/J (NOD/SCID) mice (Jackson Labs, Bar Harbor, Me., USA; 4 mice per group) were intraperitoneally injected with a maximum dose of 10 mg/kg of Brusatol, #15 or #26 compound every other day (three times per week). The survival rate was recorded to monitor the tolerance of the compounds in these mice.

### Statistical Analysis

GraphPad Prism was used for statistical analysis. The mean values with standard deviation (SD) were presented in this study when appropriate. The significance of differences was calculated by performing a 2-tailed student's t-test. The P-value of <0.05 was considered as statistically significant results. NS, not significant; * P-value <0.05; ** P-value <0.01; *** P-value <0.001; **** P-value <0.0001.

### Compound Formulations for IV/PO Administration in CD1 Mice

### Brusatol Formulation (IV/PO)

Brusatol (0.56 mg) was added to 0.933 mL of 20% HPBCD with vortex and sonication to obtain the IV solution (0.6 mg/mL).

Brusatol (1.31 mg) was added to 1.310 mL of 20% HPBCD with vortex and sonication to obtain the PO solution (1 mg/mL).

### Compound 15 Formulation (IV/PO)

Compound 15 (0.72 mg) was added to 1.200 mL of 20% HPBCD with vortex and sonication to obtain the IV solution (0.6 mg/mL).

Compound 15 (1.21 mg) was added to 1.210 mL of 20% HPBCD with vortex and sonication to obtain the PO solution (1 mg/mL).

### Compound 26 Formulation (IV/PO)

Compound 26 (0.67 mg) was added to 0.939 mL of 20% HPBCD with vortex and sonication to obtain the IV solution (0.6 mg/mL).

Compound 26 (1.69 mg) was added to 1.421 mL of 20% HPBCD with vortex and sonication to obtain the PO solution (1 mg/mL).

### Dose Formulation Validation (Compounds 1, 15, and 26)

### Sample Preparation

The desired serial concentrations of working solutions were achieved by diluting stock solution of analyte with 50% acetonitrile in water solution. 3 μL of working solutions (5, 10, 20 50, 100, 500, 1000, 5000, 10000, 20000 ng/mL) were added to 30 μL of the blank CD1 mouse blood to achieve calibration standards of 0.5˜2000 ng/mL (0.5, 1, 2, 5, 10, 50, 100, 500, 1000, 2000 ng/mL) in a total volume of 33 μL. Five quality control samples at 1 ng/mL, 2 ng/mL, 100 ng/mL, 800 ng/mL and 1600 ng/mL for blood were prepared independently of those used for the calibration curves. These QC samples were prepared on the day of analysis in the same way as calibration standards.

33 μL of standards, 33 μL of QC samples and 33 μL of unknown samples (30 μL of blood with 3 μL of blank solution) were added to 200 μL of acetonitrile containing IS mixture for precipitating protein respectively. Then the samples were vortexed for 30 s. After centrifugation at 4 degree Celsius, 4000 rpm for 15 min, the supernatant was diluted 3 times with water. 5 μL or 20 μL of diluted supernatant was injected into the LC/MS/MS system for quantitative analysis.

### Analytical Method

HPLC: SHIMADZU (LC-30AD, Serial No. L20555408840 AE and L20555408790 AE; DGU-20A54 Serial No. L20705415563 IX; CBM-20A Serial No. L20235430897 CD; SIL-30AC Serial No. L20565404174 AE; CTO-30A, Serial No. L20575401123 CD; Rack Changer II, Serial No. L20585400944 SS; Column: Phenomenex Kinetex 5u EVO C18 (50×2.1 mm); LCMS-8060 instrument (Serial No. 011105400283 AE); Mobile phase—solution A: 5% acetonitrile in water (0.1% formic acid), solution B: 95% acetonitrile in water (0.1% formic acid); Flow rate: 0.5 mL/min; Injection volume: 5 or 20 μL.

TABLE 1
Solvent gradient for analytical method

| Time (min) | solvent A (%) | solvent B (%) |
|---|---|---|
| 0.01 | 90.0 | 10.0 |
| 0.20 | 90.0 | 10.0 |
| 1.70 | 0.00 | 100 |
| 2.10 | 0.00 | 100 |
| 2.11 | 90.0 | 10.0 |
| 2.50 | 90.0 | 10.0 |

TABLE 2
Dose formulation validation (1)

| | Measured | Mean | Accuracy | SD | CV |
|---|---|---|---|---|---|
| Route | (mg/mL) | (mg/mL) | (%) | (mg/mL) | (%) |
| IV | 0.598 | 0.573 | 95.4 | 0.070 | 12.2 |
| IV | 0.493 | 0.573 | 95.4 | 0.070 | 12.2 |
| IV | 0.626 | 0.573 | 95.4 | 0.070 | 12.2 |
| PO | 0.994 | 0.994 | 96.4 | 0.027 | 2.81 |
| PO | 0.955 | 0.955 | 96.4 | 0.027 | 2.81 |
| PO | 0.943 | 0.943 | 96.4 | 0.027 | 2.81 |

TABLE 3
Dose formulation validation (15)

| | Measured | Mean | Accuracy | SD | CV |
|---|---|---|---|---|---|
| Route | (mg/mL) | (mg/mL) | (%) | (mg/mL) | (%) |
| IV | 0.608 | 0.616 | 103 | 0.008 | 1.23 |
| IV | 0.618 | 0.616 | 103 | 0.008 | 1.23 |
| IV | 0.623 | 0.616 | 103 | 0.008 | 1.23 |
| PO | 1.07 | 1.08 | 108 | 0.02 | 2.12 |
| PO | 1.06 | 1.08 | 108 | 0.02 | 2.12 |
| PO | 1.10 | 1.08 | 108 | 0.02 | 2.12 |

TABLE 4
Dose formulation validation (26)

| | Measured | Mean | Accuracy | SD | CV |
|---|---|---|---|---|---|
| Route | (mg/mL) | (mg/mL) | (%) | (mg/mL) | (%) |
| IV | 0.680 | 0.669 | 111 | 0.010 | 1.49 |
| IV | 0.660 | 0.669 | 111 | 0.010 | 1.49 |
| IV | 0.666 | 0.669 | 111 | 0.010 | 1.49 |
| PO | 1.16 | 1.17 | 117 | 0.03 | 2.58 |
| PO | 1.16 | 1.17 | 117 | 0.03 | 2.58 |
| PO | 1.21 | 1.17 | 117 | 0.03 | 2.58 |

### EXAMPLES

Various embodiments of the present application can be better understood by reference to the following Examples which are offered by way of illustration. The scope of the present application is not limited to the Examples given herein.

### Example 1: The Quassinoid Compound Brusatol Inhibits Viability of Hematologic Malignancy Derived Cells

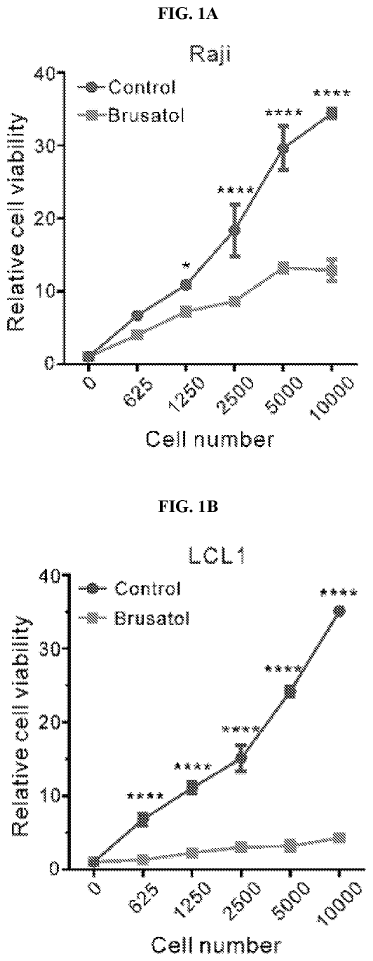

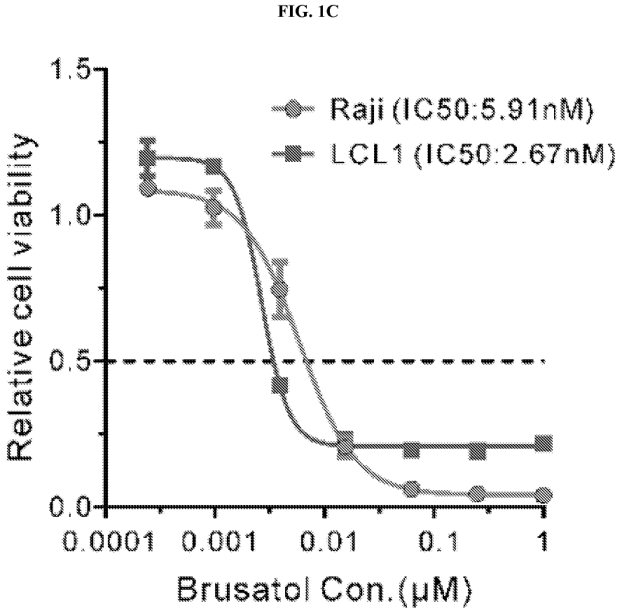

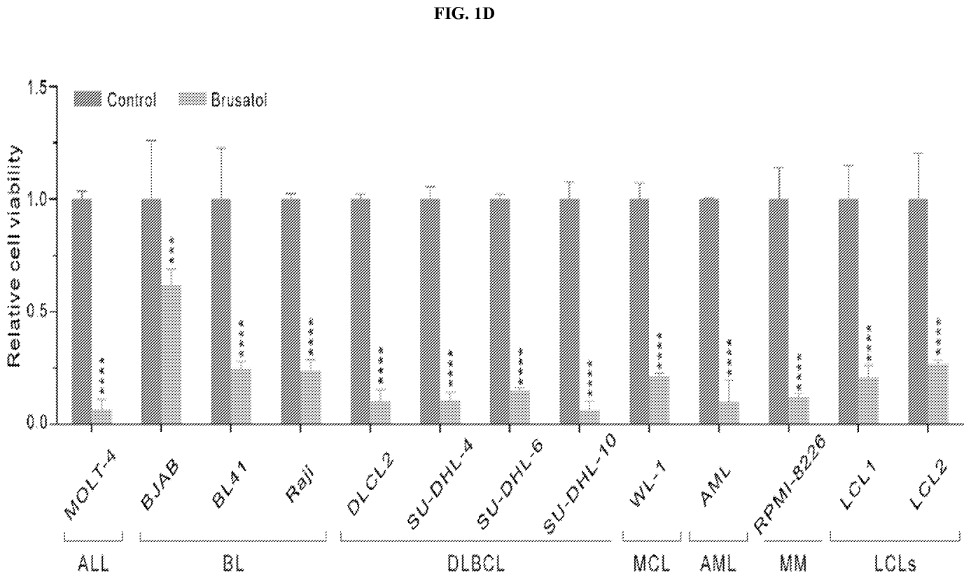

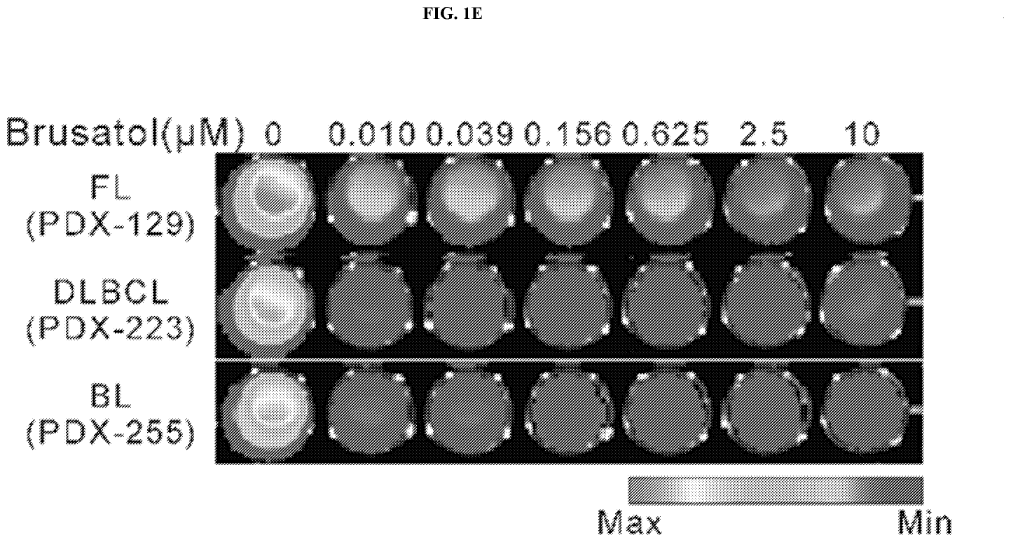

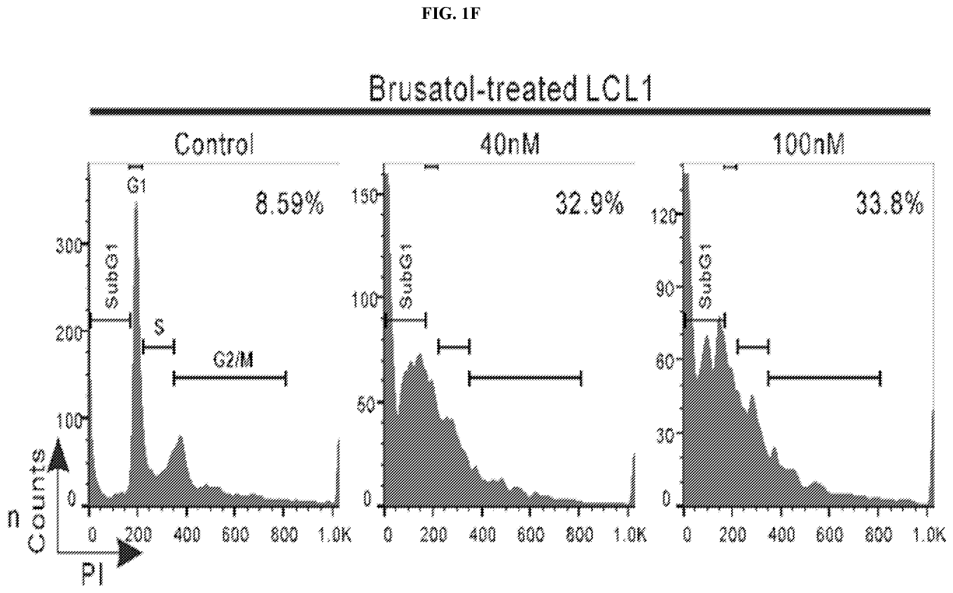

Brusatol has been previously demonstrated to show inhibitory activities towards the growth of some leukemias. To investigate its effects on EBV-associated lymphomas and other types of lymphomas, the viability of a Burkitt's lymphoma (BL) cell line Raji and another Epstein Barr virus (EBV)-transformed lymphoblastoid cell line (LCL1) were monitored when treated with Brusatol. Results showed that Brusatol effectively inhibited the growth of the BL cell line Raji even at increasing cell concentrations (FIG. 1A). Furthermore, it also exhibited strong inhibitory effects on the EBV-transformed LCL1 cells (FIG. 1B). The half-maximal inhibitory concentration (IC₅₀) was determined for both Raji and LCL1 cells. The results showed an effective IC₅₀ of between 2 nM and 6 nM for both cell lines (FIG. 1C).

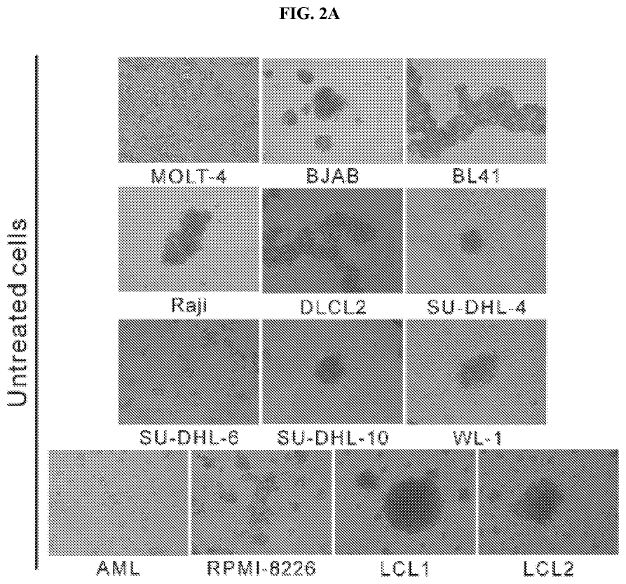

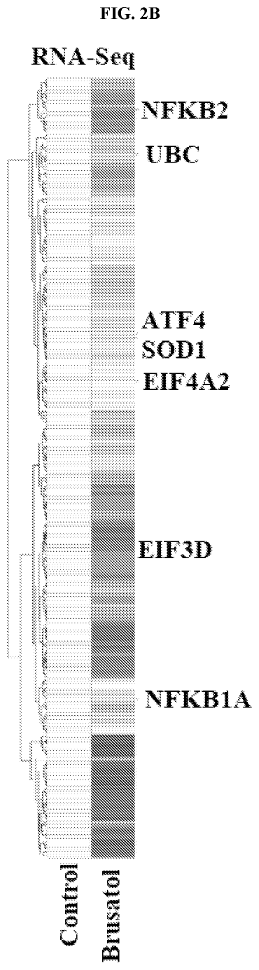

To determine the ability of Brusatol to inhibit other types of lymphomas and leukemias, its inhibition of a number of different human hematologic malignancies was evaluated in various cell lines (Table 5, FIG. 2A). The results demonstrated that Brusatol had broad and potent inhibitory effects on the majority of these cell lines in addition to EBV-associated lymphomas, which include lymphoma, leukemia, and multiple myeloma cell lines (FIG. 1D). Brusatol also inhibited hematologic malignant cells obtained from three independent patient-derived xenografts (PDX) (FIG. 1E). These results provide new evidence as to the clinical potential of Brusatol for treatment of a broad range of hematologic malignancies. Furthermore, cell cycle analyses showed that Brusatol strongly disrupted the cell cycle phases, and induced cell death 72 hours post-treatment, which was consistent with the results of cell viability assays in EBV-transformed LCLs (FIG. 1F). Its inhibitory effects were associated with inhibition of cell cycle progression with a 3.5 to 4-fold increase in subG1 population in cells treated with Brusatol (FIG. 1F). These studies demonstrated that Brusatol inhibited the growth of EBV-positive as well as multiple hematologic malignant cell lines in vitro, and has strong potential as a candidate small molecule therapeutic agent for treatment of hematologic malignancies.

TABLE 5
Cell lines used in the present disclosure

| Cells | Organism | Cell type/Disease |
|---|---|---|
| AML | human | Monocyte, acute myeloid leukemia |
| BJAB | human | B lymphocyte, Burkitt's lymphoma |
| BL41 | human | Burkitt's lymphoma |
| DLCL2 | human | Diffuse large B-cell lymphoma germinal center B-cell type, B-cell |
| HepG2 | human | Hepatocellular carcinoma |
| HL-60 | human | Promyeloblast, acute promyelocytic leukemia |
| H929 | human | B lymphocyte, plasmacytoma, myeloma |
| K562 | human | Chronic myelogenous leukemia (CML) |
| LCL1 | human | Epstein-Barr virus-transformed lymphoblastoid cell lines |
| LCL2 | human | Epstein-Barr virus-transformed lymphoblastoid cell lines |
| MOLM14 | human | Acute myeloid leukemia |
| MOLT-4 | human | T lymphoblast, acute lymphoblastic leukemia |
| NB-4 | human | Acute promyelocytic leukemia |
| PBMC | human | Primary Peripheral Blood Mononuclear Cells, normal |
| Raji | human | B lymphocyte, Burkitt's lymphoma |
| RPMI-8226 | human | Plasma cell myeloma, B lymphocyte, plasmacytoma |
| SU-DHL-4 | human | Diffuse large B-cell lymphoma germinal center B-cell type, B |
| SU-DHL-6 | human | Diffuse large B-cell lymphoma germinal center B-cell type, B |
| SU-DHL-10 | human | Diffuse large B-cell lymphoma, B lymphocyte, large cell |
| WL-1 | human | Mantle cell lymphoma |
| Toledo | human | B lymphocyte, diffuse large cell lymphoma; non-Hodgkin's B cell |
| Preiffer | human | Diffuse large cell lymphoma; non-Hodgkin’s B cell lymphoma |
| Jeko-l | human | Mantle Cell lymphoma |
| PDX129 | human | Cutaneous lymphoma |
| PDX223 | human | Diffuse large B cell lymphoma |
| PDX255 | human | Burkitt’s lymphoma |

### Example 2: Brusatol Specifically Targets the PI3K/AKT Signaling Pathway

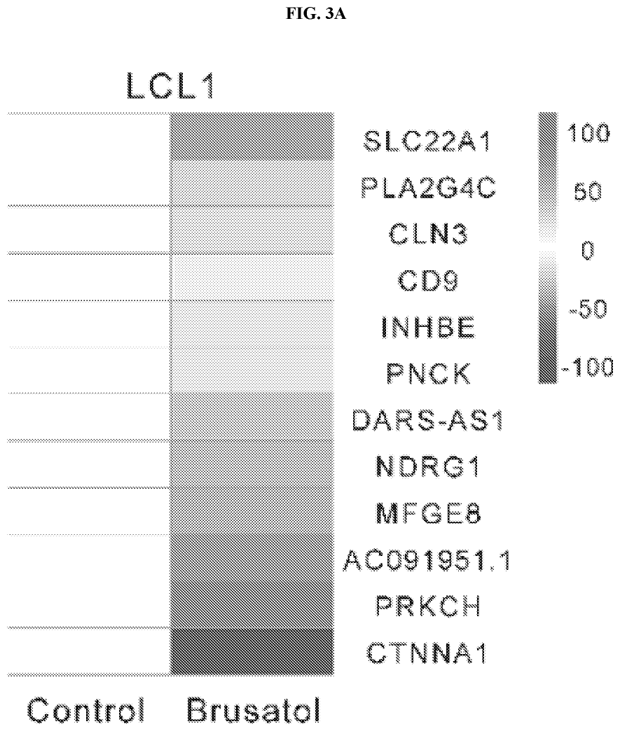

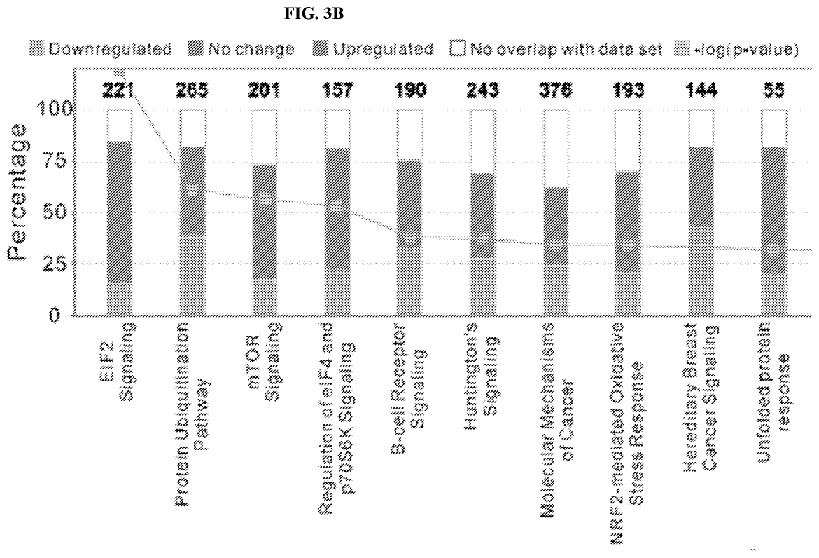

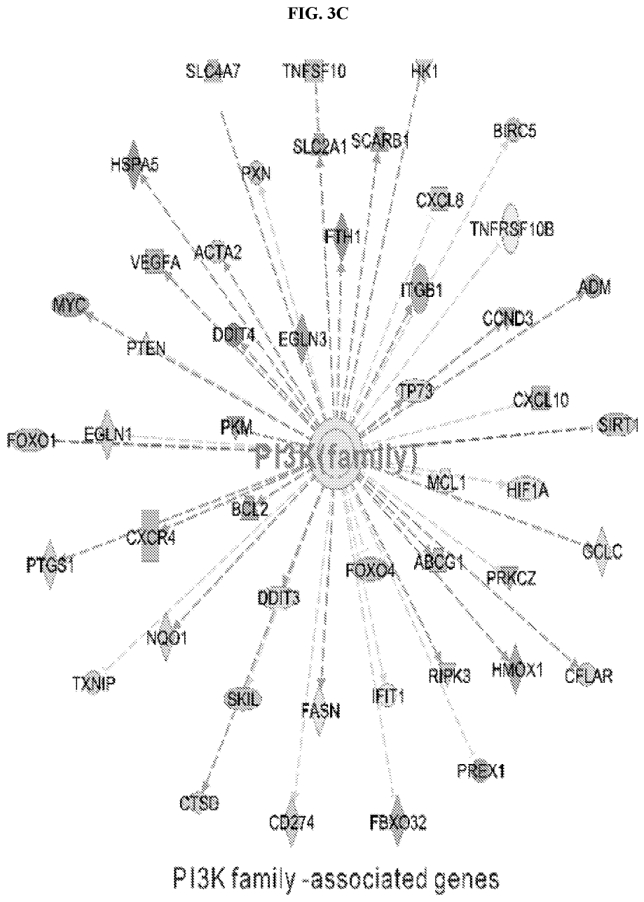

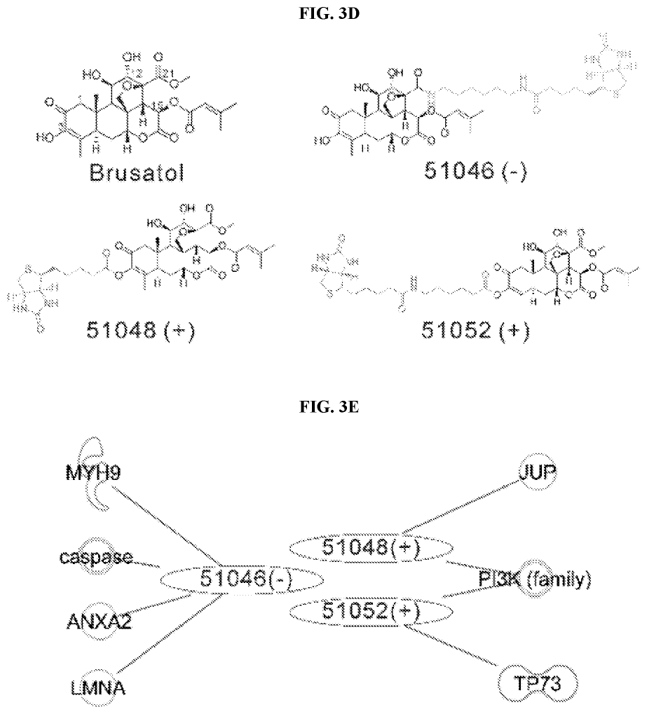

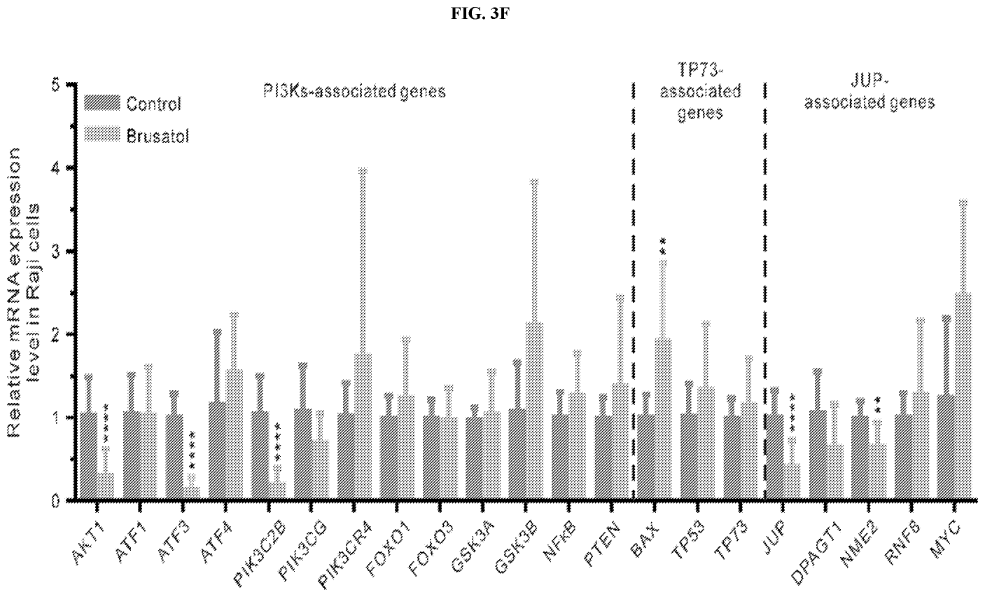

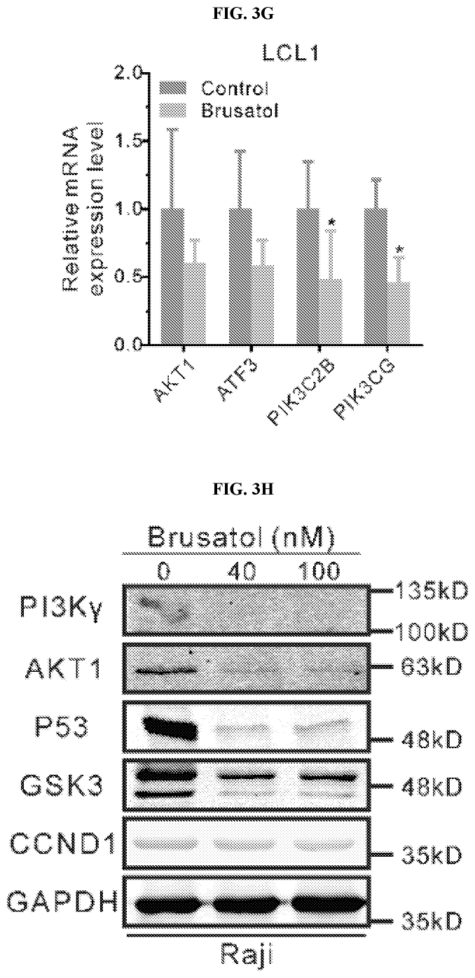

Brusatol exhibited significant inhibition of the hematologic malignancies derived cell lines. However, the mechanism of its anti-cancer activities has not been previously explored. To address this, RNA-Seq analysis was performed with Brusatol-treated EBV-transformed LCLs to characterize the transcription reprogramming events. Analysis of the results showed that SLC22A1, a twelve-membrane cation transporter, was highly upregulated and may be linked to the elimination of Brusatol. CTNNA1, the catenin family member involved in the connection of cadherin to actin filament, was dramatically downregulated. Twelve genes were shown as the most significantly altered from the transcription analyses described herein (FIG. 3A). Other significant genes involved in a broad range of cellular pathways were also affected (Table 6 and FIG. 2B). The regulated genes and their associated pathways are shown (FIGS. 2A and Table 6). Specifically, the results showed that EIF2 signaling, protein ubiquitination, and mTOR signaling, as well as regulation of eIF4 and p70S6K signaling, were critical pathways involved in Brusatol-mediated inhibition (Table 6 and FIG. 3B). These pathways are tightly associated with the regulation of protein synthesis. Therefore, it is reasonable that Brusatol was recognized as an inhibitor of protein synthesis. Additionally, the NRF2-mediated oxidative stress response was also linked to Brusatol treatment (FIG. 3B). This supports a previous study which showed that Brusatol inhibited the NRF2 pathway. NF-κB and c-Myc-associated pathways were also previously linked to Brusatol-associated inhibition. However, no definitive results have supported their role as direct effectors of Brusatol inhibition although it is certainly possible that they may have an indirect role.

TABLE 6
P-value and overlap percent of Brusatol-related signaling pathways

| Name | p-value | Overlap |
|---|---|---|
| EIF2 signaling | 1.05E−35 | 65.2% (144/221) |
| Protein ubiquitination pathway | 1.02E−18 | 50.6% (134/265) |
| mTOR signaling | 2.81E−17 | 53.2% (107/201) |
| Regulation of eIF4 and p70S6K signaling | 2.83E−16 | 56.1% (88/157) |
| B-cell receptor signaling | 6.40E−12 | 48.4% (92/190) |

To explore the results of the RNA-Seq, upstream regulator analysis using the Ingenuity Pathway Analysis (IPA) program was performed. Analysis of the mRNA profile of Brusatol-treated cells identified critical upstream regulators, which include specific PI3K family members, JUP, TP73, and TP53 (FIG. 3C). These are also potential targets of Brusatol and are likely involved in regulation of downstream signaling to promote Brusatol-mediated inhibition. However, additional biochemical data will further provide evidence of the interaction with these candidates.

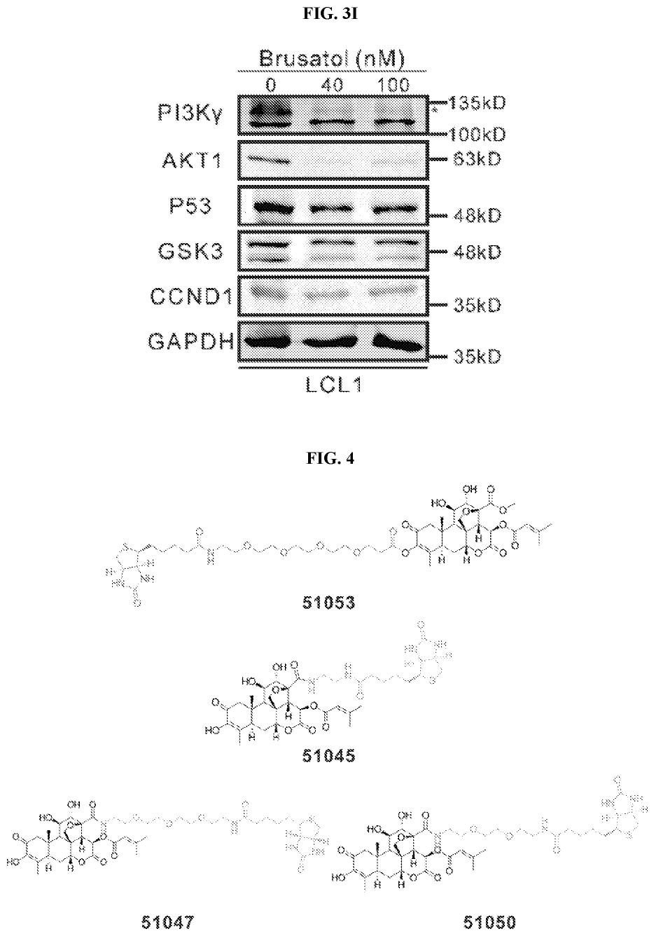

To definitively identify the direct targets of Brusatol, a series of biotin-conjugated Brusatol derivatives were synthesized to capture its targets using mass spectrometry (MS) analysis (FIG. 3D, and FIG. 4). The structure-activity relationship (SAR) was determined to identify positions that allowed for the chemical conjugation of biotin without affecting its biochemical activity. Large linear esters at C-21 led to a reduction in potency, but the incorporation of nitric oxide-releasing groups at C-3 was better tolerated and retained activity. Thus, the biotin-conjugated Brusatol analogs derived from positions C-3 and C-21 were examined to demonstrate their IC₅₀ on multiple PDXs, and lymphoma cell lines (FIG. 4). The results showed that the C-3 hydroxyl on Brusatol can be derivatized with a lipophilic tert-butoxy carbonyl (Boc) protected glycine or hydrophilic smaller amino acids such as beta-aminobutyric acid and proline, with minimal effects on its inhibitory ability, and was used to attach the biotin moiety. In contrast, a bigger isopropyl ester at C-21 resulted in a loss of activity (Table 7). Two additional amide derivatives from C-21 lost activity, indicating that a large appendant is not tolerated at this position (FIG. 4). Therefore, the active domain of Brusatol is associated with the C-21 position.

TABLE 7
IC₅₀ assays of biotin-conjugated compounds
in multiple PDXs and lymphoma cells

| IC₅₀ | Modified at C-3 position | | | Modified at C-21 position | | | |
|---|---|---|---|---|---|---|---|
| (μM) | 51048 | 51052 | 51053 | 51045 | 51046 | 51047 | 51050 |
| PDX129 | 1.726 | 0.076 | 0.656 | >10 | 1.637 | >10 | >10 |
| PDX223 | 3.565 | 0.108 | 1.056 | >10 | 8.974 | >10 | >10 |
| Toledo | >10 | 0.388 | >10 | >10 | 0.494 | >10 | >10 |
| Pfeiffer | 3.170 | 0.079 | 0.414 | >10 | 8.913 | >10 | >10 |
| Jeko-1 | 1.186 | 0.028 | 1.32 | >10 | 3.013 | >10 | >10 |

Next, C-3-biotinylated Brusatol derivatives were synthesized with different linkers (51048, 51052) and a C-21 biotin conjugate as a negative control (51046). These three biotin-conjugated Brusatol derivatives were selected for mass spectrometry (MS) experiments (FIG. 3D). Approximately 30 proteins were identified as potential targets for each derivative. To narrow down the candidates, the MS results were integrated with upstream regulators identified from the RNA-Seq analysis. Strikingly, PI3K family members were the only candidates identified with both 51048 and 51052 biotin-conjugated derivatives (FIG. 3E). Compound 51048 interacted with PIK3CG and JUP encoded proteins, while 51052 interacted with PIK3C2B and TP73 encoded proteins. PIK3CG encodes PI3Kγ and PIK3C2B encodes PI3KC2β, both members of the PI3K family. JUP encodes for the plakoglobin protein, known as junction plakoglobin or γ-catenin, which is important in acute myeloid leukemia (AML). The P73 protein is also widely studied in association with hematologic malignancies.

To validate these links of upstream regulators, their downstream associated genes were identified and the mRNA transcripts were monitored using Real-time PCR in Raji cells treated with Brusatol. The mRNA levels of PI3K family-associated AKT1, ATF3, PIK3C2B; TP73-associated BAX; and JUP-associated JUP and NME2 were significantly down-regulated after Brusatol treatment (FIG. 3F). These may all be potential targets of Brusatol. However, focus was placed on the PI3K family members, PI3Kγ and PI3KC2β, as they were the only two proteins precipitated by both active biotin-conjugated Brusatol derivatives (FIG. 3E). Moreover, a primary focus was to determine if Brusatol regulated key members of the PI3Ks signaling pathways. The results demonstrated that AKT1, ATF3, PIK3CG, and PIK3C2B were downregulated at the mRNA levels in Brusatol treated EBV-positive Raji and LCL1 cells (FIGS. 3F-3G). The levels of AKT1 and GSK3 proteins were also decreased on Brusatol treatment (FIGS. 3H-3I). This provided strong supporting evidence that Brusatol can target PI3K family members to perturb the PI3K/AKT signaling pathway.

### Example 3: PI3K Proteins are Crucial for Brusatol-Mediated Inhibitory Effects

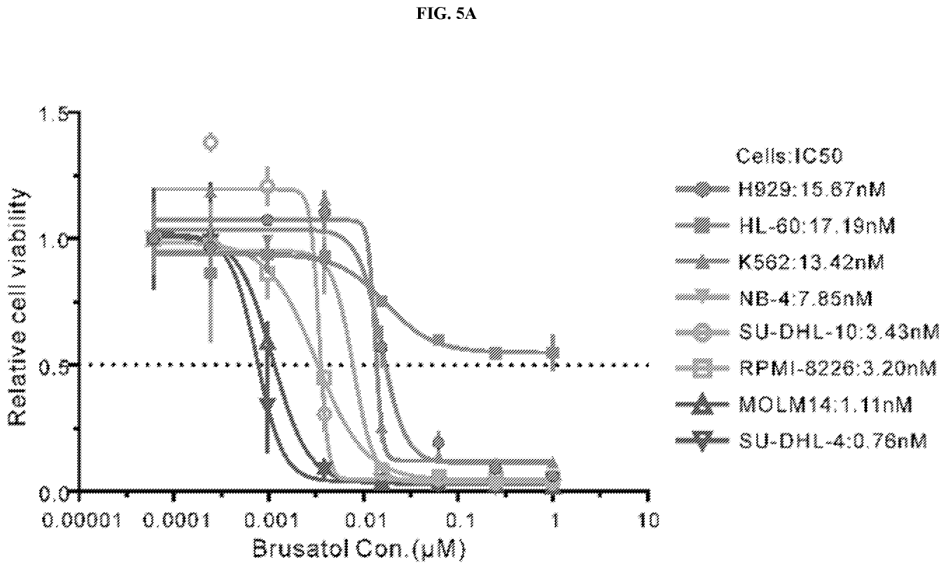

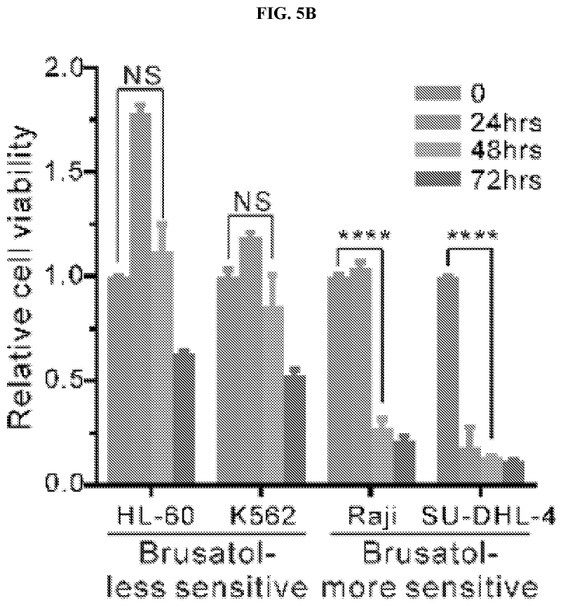

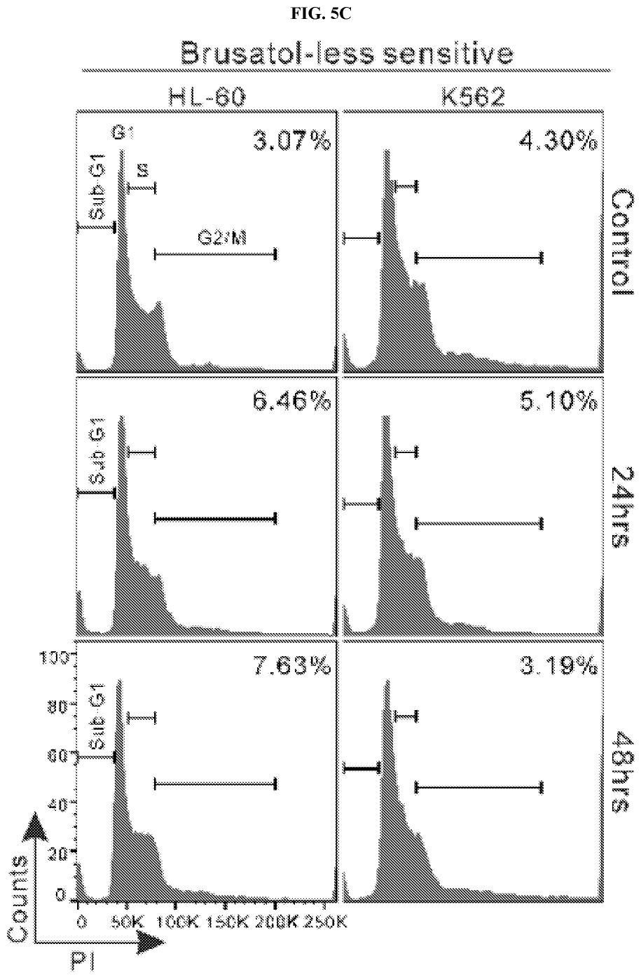

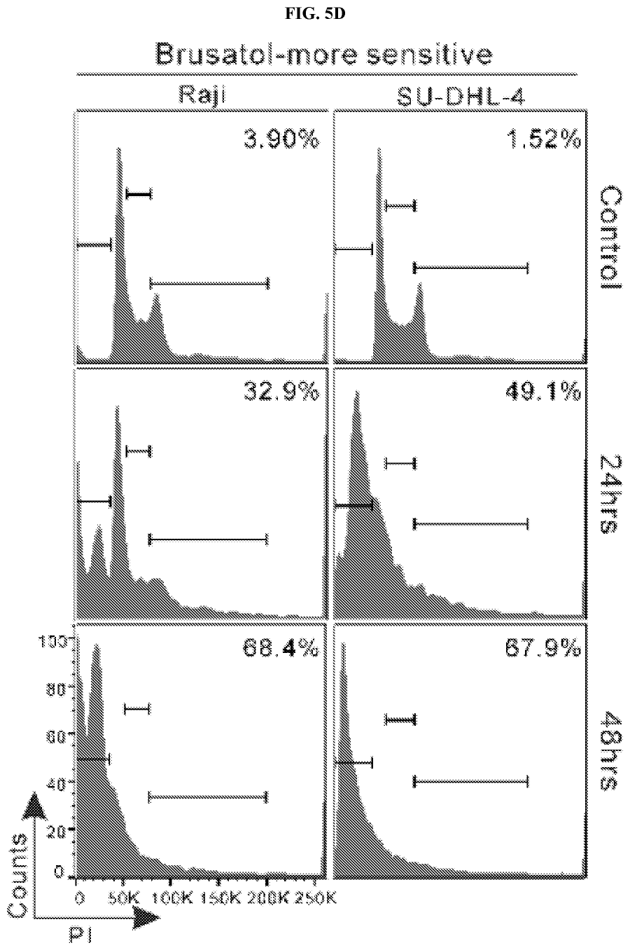

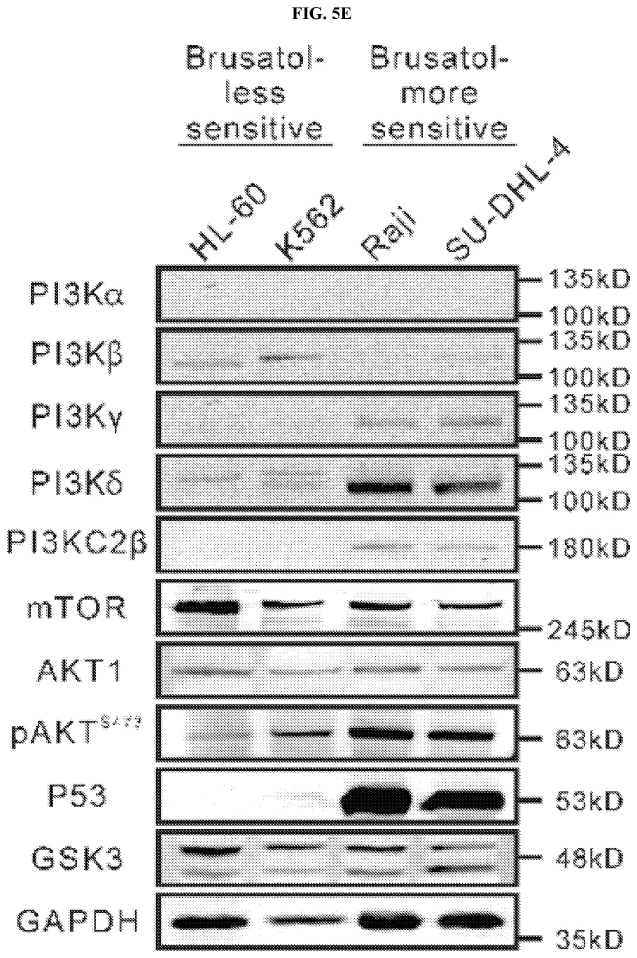

To identify the mechanism by which Brusatol targets the PI3K/AKT pathway in hematologic malignancies, several cell lines derived from different types of hematologic malignancies with varying levels of PI3Ks were screened. Notably, the IC50 showed that HL60 and K562 cell lines had reduced sensitivity to Brusatol treatment when compared to MOLM14 and SU-DHL-4 cell lines, which were about 10-fold more sensitive to Brusatol treatment at 72 hours (FIG. 5A). Furthermore, Brusatol exhibited substantially lower inhibition of HL-60 and K562 cell lines compared to Raji and SU-DHL-4 cell lines (FIG. 5B). Specifically, at even 48 hours post-treatment, Brusatol inhibited the viability of more than 80% of the Raji and SU-DHL-4 cells. However, the inhibitory effects were lower on HL-60 and K562 cell lines (FIG. 5B). Additionally, cell cycle analyses demonstrated that HL-60 and K562 cells were less sensitive than Raji and SU-DHL-4 cells. After 48 hours of treatment with Brusatol, 68.4% of Raji cells and 67.9% of SU-DHL-4 cells were in the sub-G1 phase. However, only 7.63% of HL-60 cells and 3.19% of K562 cells were in a similar phase (FIG. 5C). Therefore, HL-60 and K562 were selected as the Brusatol-less sensitive set of cells, and Raji and SU-DHL-4 as the Brusatol-more sensitive set of cells for further examination.

To compare the expression of PI3K family members and related molecules, the endogenous levels in the two sets of cell lines (Brusatol-less or -more sensitive cells) were determined. It was demonstrated that cell lines with reduced levels of PI3Kγ and PI3KC2β were less sensitive to Brusatol treatment (FIG. 5D). Interestingly, along with PI3Kγ, PI3Kδ and P53 were also highly expressed in the two Brusatol-more sensitive cell lines (FIG. 5D). Further, Brusatol-less sensitive or Brusatol-more sensitive sets were incubated with increasing concentrations (0, 50 nM, 100 nM) of Brusatol at three different time points (0, 12 hours, 24 hours). The results showed that the protein expression of AKT1, GSK3, and mTOR was significantly reduced in Raji and SU-DHL-4 cells (Brusatol-more sensitive), and minimally affected in HL-60 and K562 cells (Brusatol-less sensitive) (FIG. 5E). This provides a possible explanation of why Raji and SU-DHL-4 cells were more sensitive and HL-60 and K562 showed less sensitivity in the cell cycle and cell viability assays. Therefore, without wishing to be bound by theory, it appears that Brusatol can specifically inhibit the PI3K/AKT/GSK3/mTOR signaling pathway.

### Example 4: The Quassinoid Family Member Brusatol can Directly Target the PI3Kγ Isoform

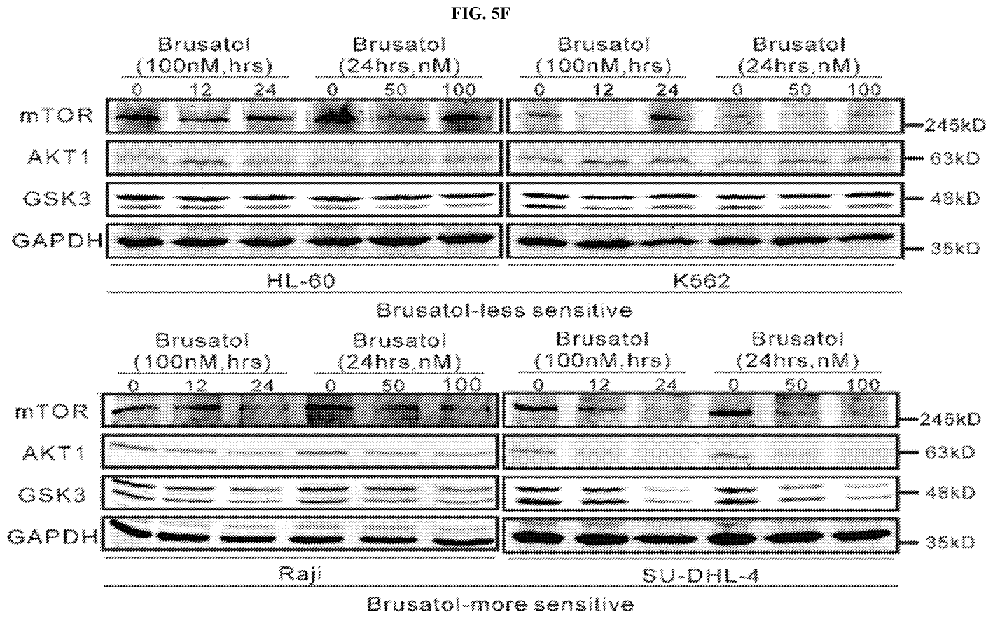

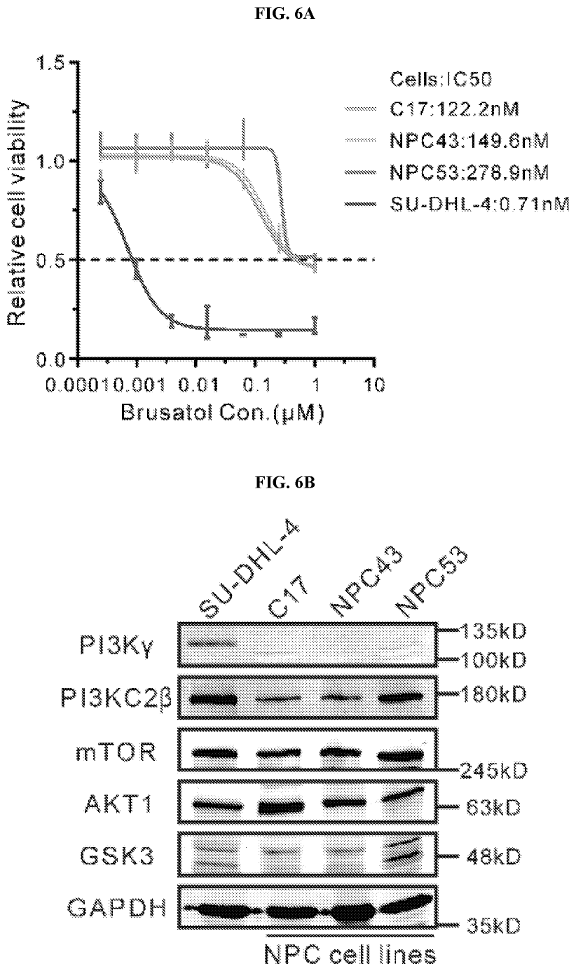

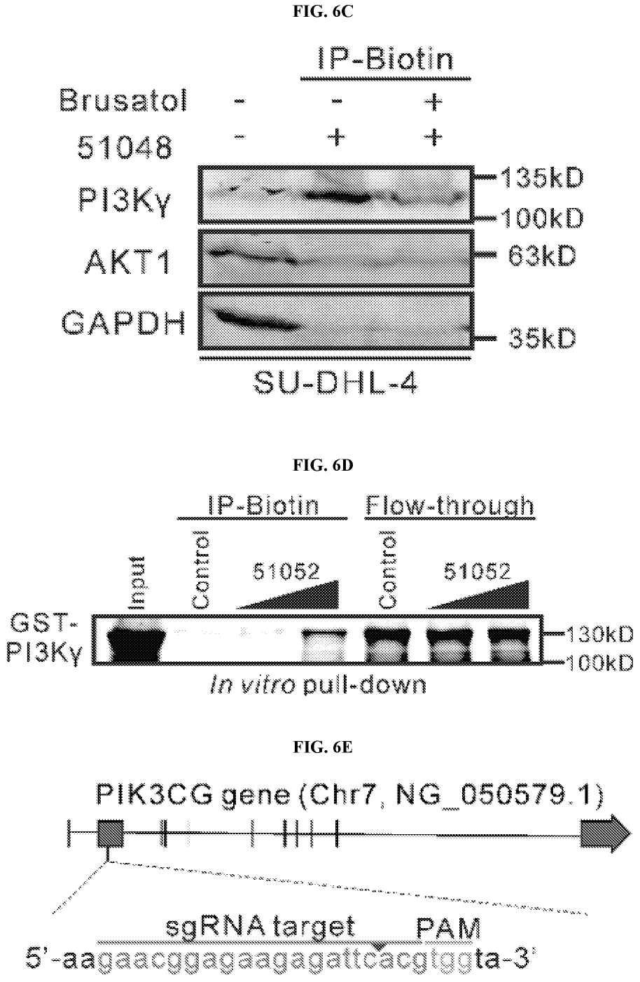

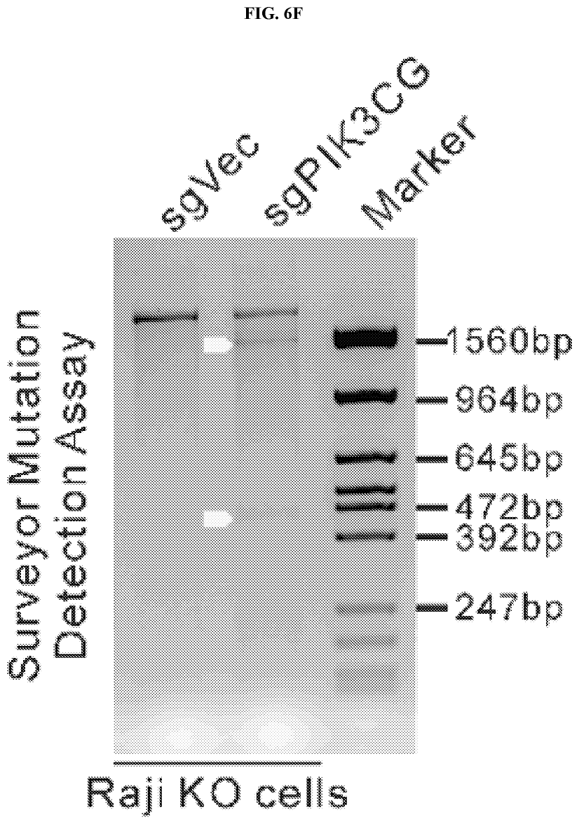

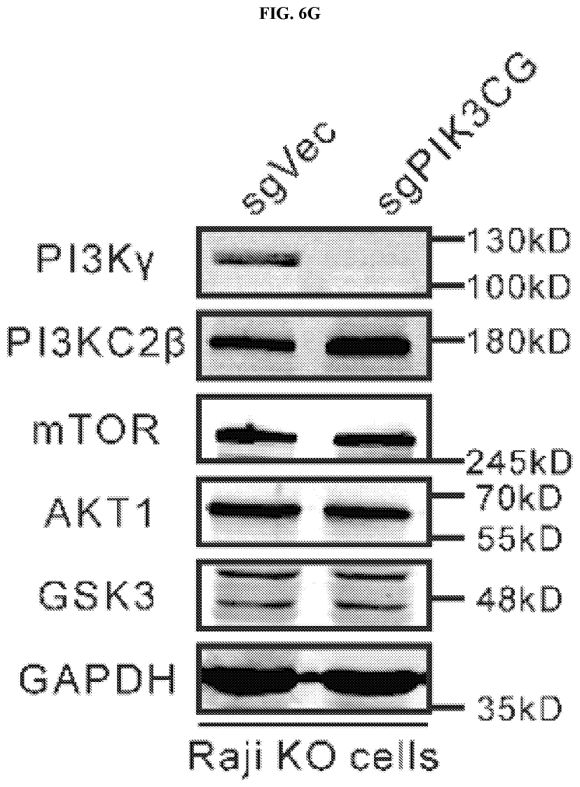

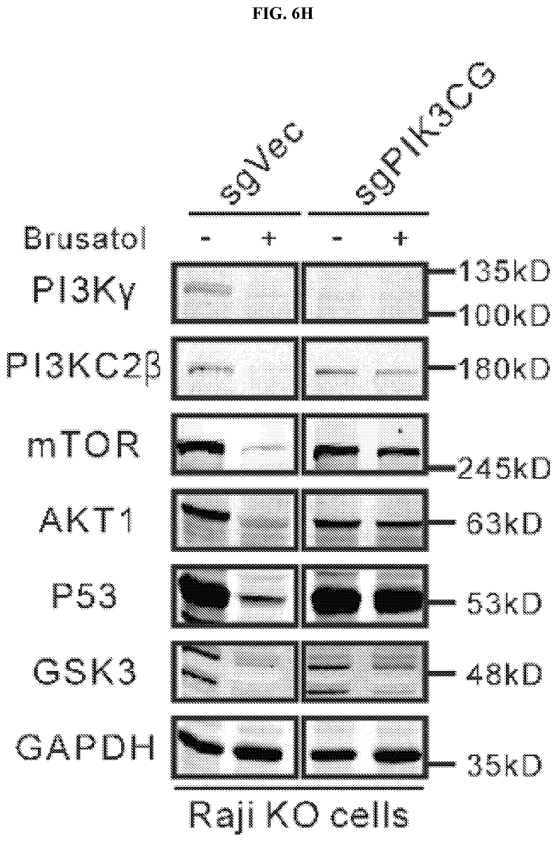

To further explore the effects of these compounds on other cancers, three patient-derived nasopharyngeal carcinoma (NPC) cell lines (C17, NPC43, and NPC53) were evaluated. Cell viability assays demonstrated that these NPC cell lines are less sensitive to Brusatol when compared to the SU-DHL-4 cell line by IC50 (FIG. 6A). Notably, these NPC cells expressed relatively high levels of PI3KC2β, but PI3Kγ was minimally detected (FIG. 6B). Therefore, these results provide additional evidence that supports PI3Kγ as a potential target of Brusatol and is a critical potential target for cancer therapies.

Next, biotin-conjugated pull-down assays were performed to examine the direct association of Brusatol with the PI3Kγ isoform. To perform the competitive binding assays, cell lysates from SU-DHL-4 cells were incubated with the biotin-conjugated Brusatol derivative (51048) alone or together with the parent Brusatol. The results showed that the binding activity of the Brusatol derivative (51048) with PI3Kγ was relatively strong but was reduced in the presence of the parent Brusatol compound (FIG. 6C). Additionally, the biotin-conjugated Brusatol derivative specifically interacted with isoform PI3Kγ, but had little or no detectable levels of AKT1 and GAPDH (FIG. 6C). Furthermore, GST-tagged PI3Kγ protein was purified to determine its association with the biotin-conjugated Brusatol derivative (51052) similar to 51048 at the C-3 position for conjugation in vitro. The pull-down assays demonstrated that GST-tagged PI3Kγ formed a complex with the biotin-conjugated Brusatol derivative (51052) in vitro (FIG. 6D).

To provide further support for these findings, sgRNA sequences were designed to target the exon of the PIK3CG gene (FIG. 6E), and generated knock-out (KO) Raji cell lines using CRISPR/Cas9 system (FIG. 6F). Notably, the knock-out of the PIK3CG gene in Raji cells did not affect expression of other downstream molecules of the PI3K/AKT pathway (FIG. 6G). However, AKT1 level showed no obvious decrease in the knock-out cell line with Brusatol treatment, suggesting that crucial downstream targets of PI3Kγ protein were specifically regulated (FIG. 6H). GSK3, mTOR, and P53 also showed less reduction in levels after Brusatol treatment in the knock-out cells (FIG. 6H). These results demonstrated that Brusatol can regulate the PI3K/AKT signaling pathway by targeting PI3Kγ to inhibit the viability of hematologic malignancies derived cancer cells.

### Example 5: Development of Novel Brusatol Analogs with Great Efficacy In Vitro and In Vivo

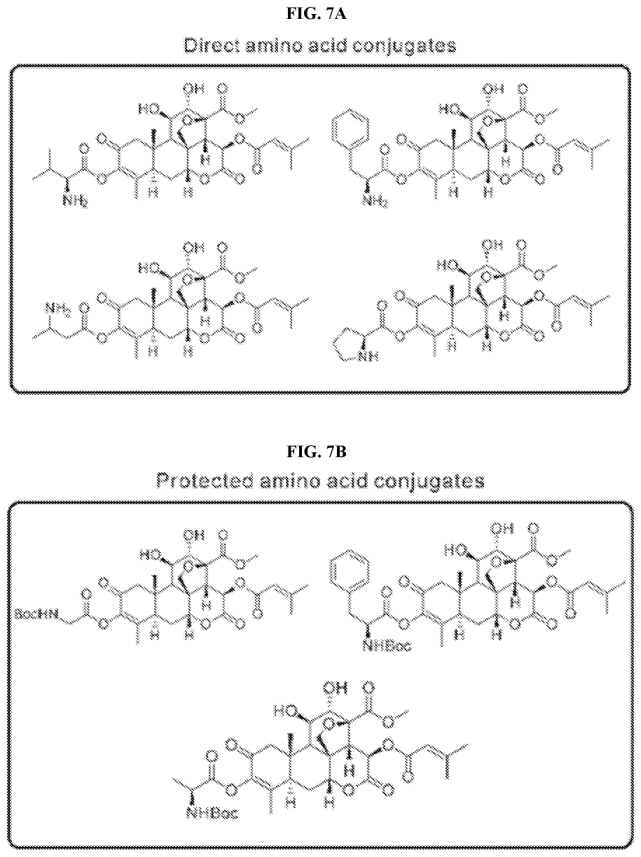

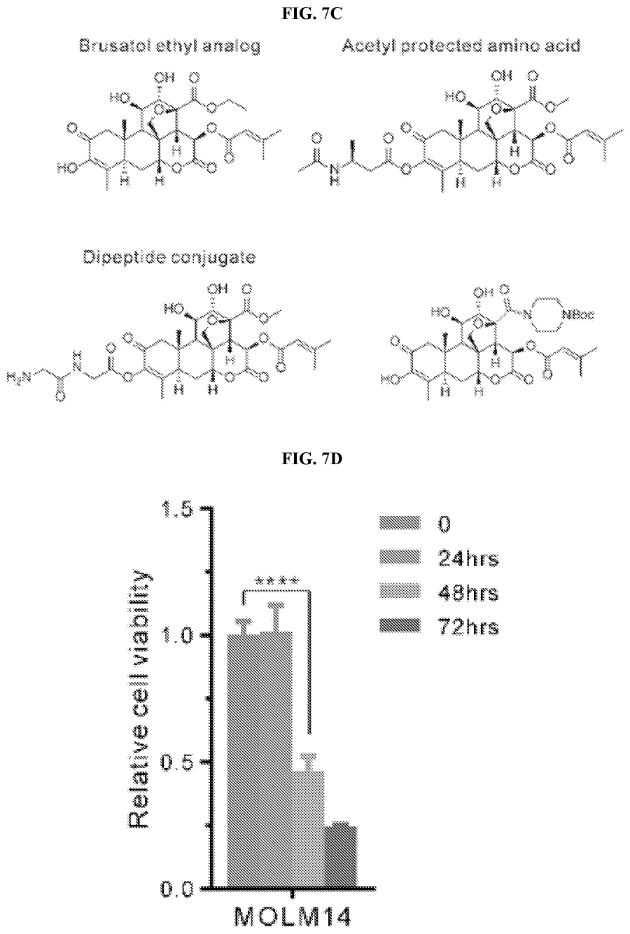

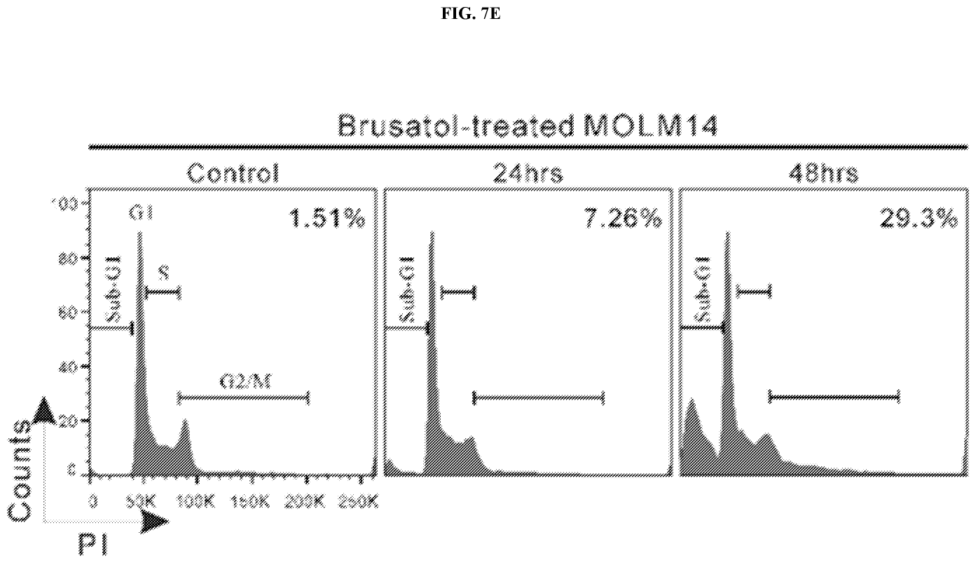

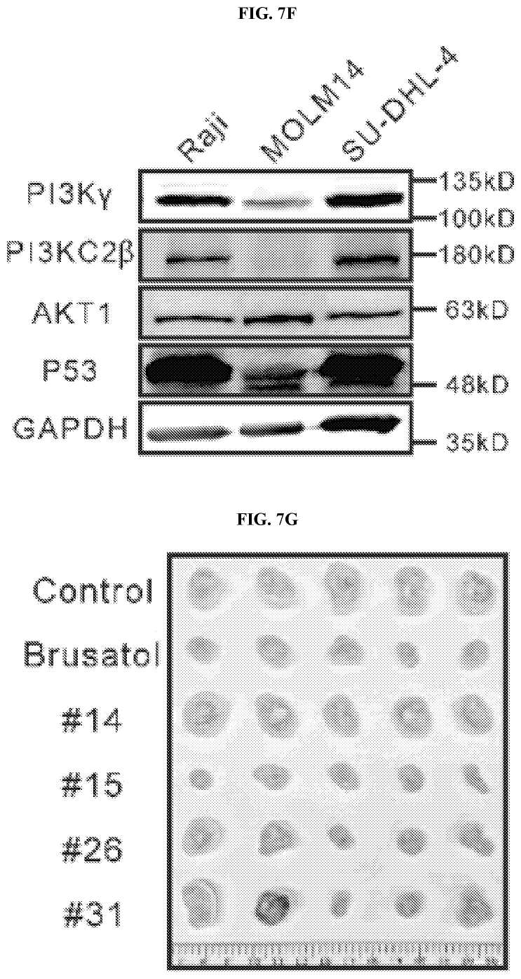

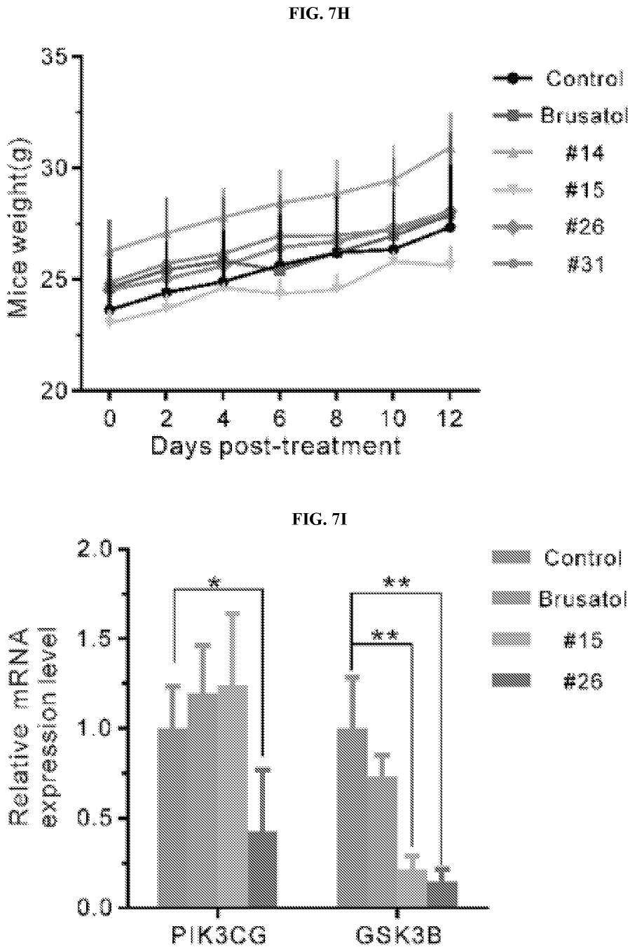

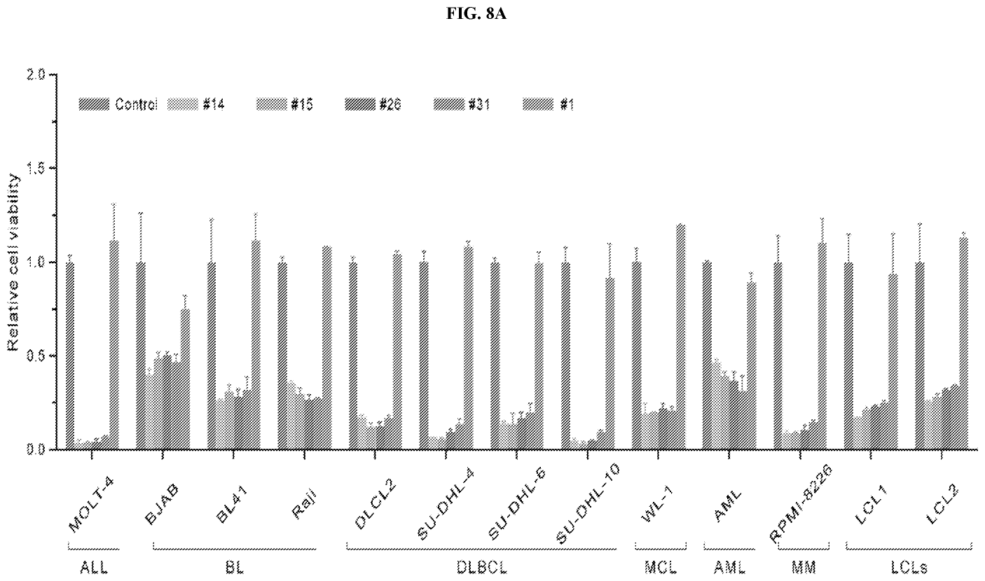

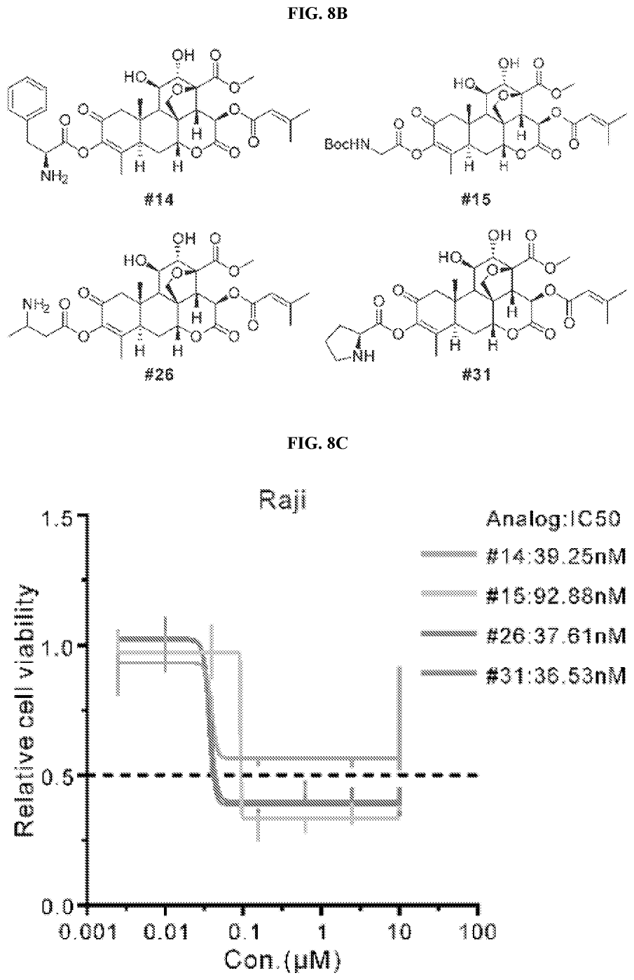

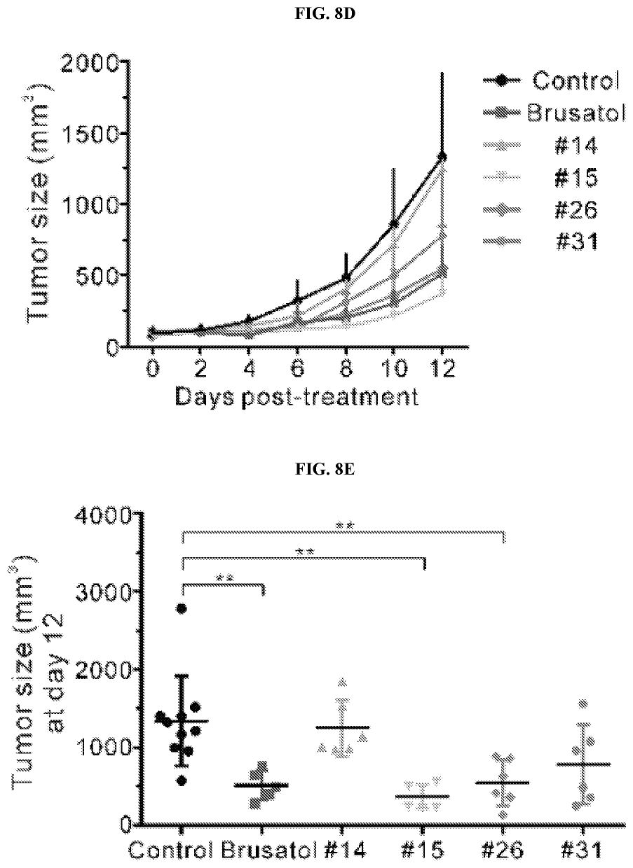

To further improve the potential of Brusatol as a therapeutic agent, a series of novel Brusatol analogs were synthesized and evaluated their activity to identify candidates with increased bioactivity and minimal toxicity. 10 selected analogs from around 70 of the strategically generated compounds maintained significant inhibitory effects on Raji cells compared to the parent Brusatol (FIGS. 7A-7C). To further investigate the effects of these active compounds, the panel of hematologic malignancy derived cell lines were used. One inactive compound #1 was set as a negative control (FIG. 7D). The results showed that four representative analogs, 14, 15, 26, and 31 were able to significantly inhibit cell viability of all 13 cell lines representing a range of hematologic malignancies (FIG. 8A). Interestingly, the selected compounds that remained active were all modified at the C-3 position (FIG. 8B), supporting the conclusion that the active domain of Brusatol is not likely to be associated with this modification (FIG. 3D and FIG. 4). These novel Brusatol analogs showed comparable efficacy in MOLT-4, SU-DHL-4, SU-DHL-10, and RPMI-8226 cells as more than 90% of viable cells were inhibited after treatment (FIG. 8A). Expectedly, the inactive compound 1 was unable to inhibit the growth of these cells in vitro (FIG. 8A). Further, the IC50 showed that these promising candidates maintained effective inhibitory effects comparable to what was previously seen above with the parent Brusatol (FIG. 8C).

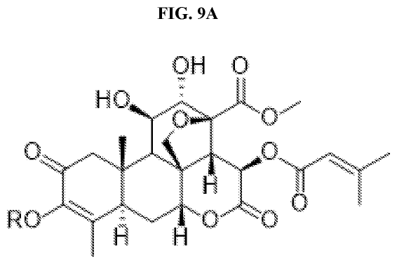

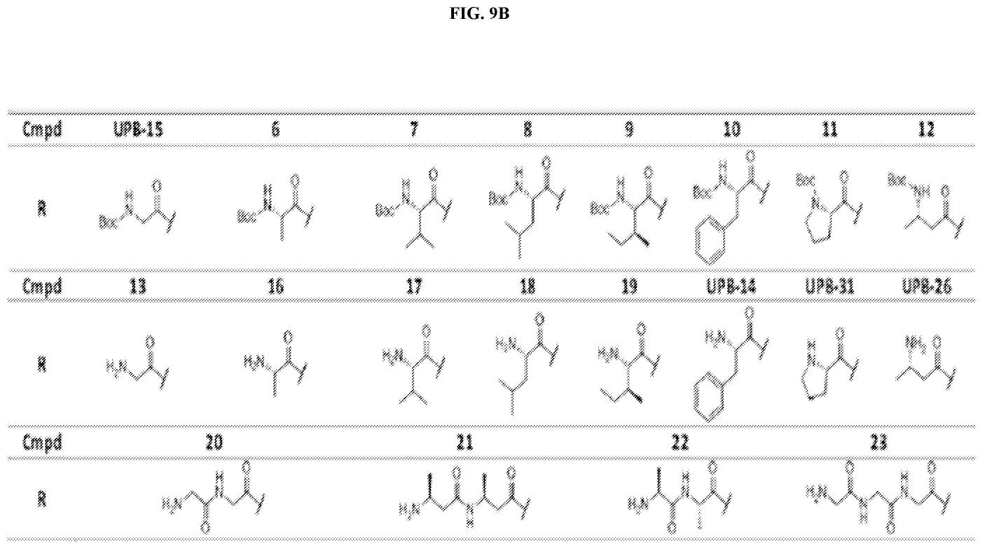

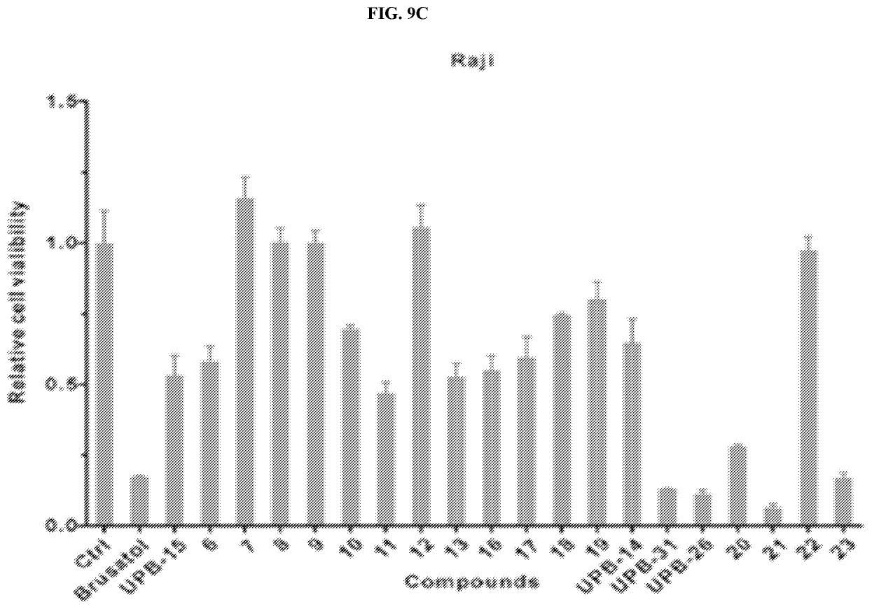

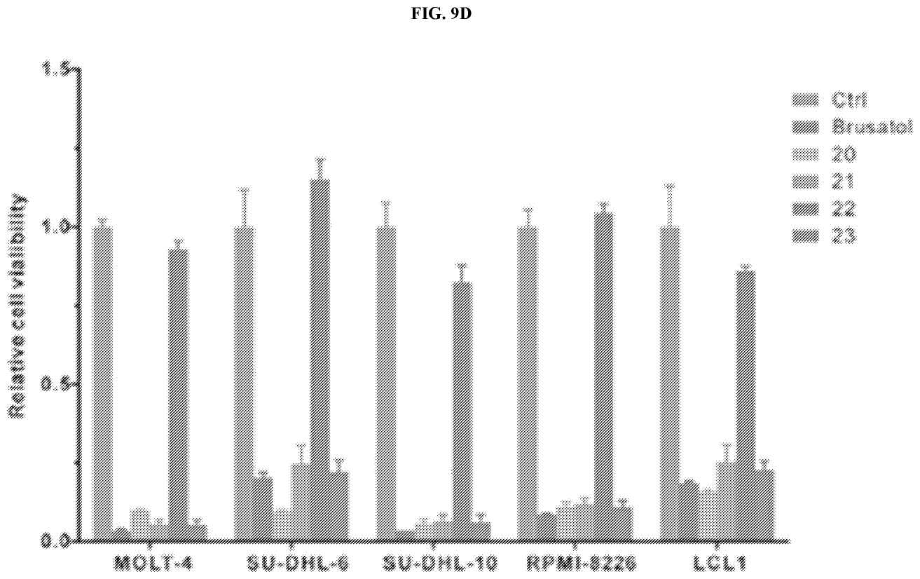

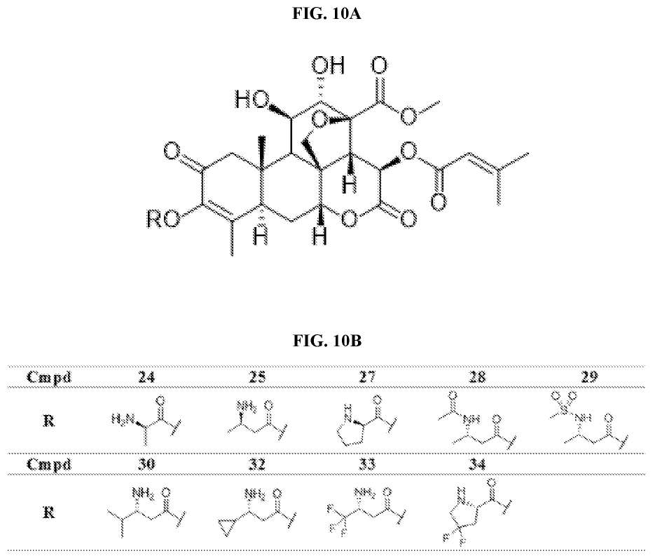

To investigate their inhibitory activities on the growth of EBV-associated lymphomas and other types of lymphomas, conjugates were first investigated for their effects on the viability of Raji cells, a Burkitt's lymphoma (BL) cell line, when treated at 100 nM for 72 h. Compounds 14, 15, 26, and 31, as described elsewhere herein, are included for comparison. As illustrated in FIGS. 9A-9D, for Boc protected amino acid conjugates, smaller amino acid esters, such as glycine 15, alanine 6, and proline 11, gave moderate inhibition, while larger amino acid esters did not show significant inhibition. In contrast, all of the unprotected amino acid conjugates demonstrated improved inhibitions over their corresponding more lipophilic Boc-protected precursors (FIG. 9C). Similarly, conjugates with smaller amino acids exhibited better potencies than those with larger amino acids. In addition to the β-homoalanine conjugate 26 and proline conjugate 31, the di-glycine peptide conjugate 20, di-homoalanine peptide conjugate 21, and tri-glycine peptide conjugate 23 also displayed potent activities comparable to or higher than the parent brusatol. Interestingly, di-alanine peptide conjugate 22 was not active. Next, the activities of these four conjugates were examined against cell lines derived from other types of human hematologic malignancies. The cell lines included: MOLT-4, acute lymphoblastic leukemia (ALL) cells; SU-DHL-6 and SU-DHL-10, diffuse large B-cell lymphoma (DLBCL) cells; RPMI-8226, multiple myeloma (MM) cells; and LCL1, an EBV-transformed lymphoblastoid cell line. These cell lines represent the cell models of a range of hematologic malignancies. The results demonstrated that the active conjugates selected from Raji cell line all had broad and potent inhibitory effects on the majority of these cell lines, including EBV-as-sociated lymphomas (LCL1). But in particular, MOLT-4 and SU-DHL-10 cells showed more sensitivity to these analogs, except 22, compared to the others (FIG. 9D).

To examine the anti-cancer effects of these Brusatol analogs in vivo, six-week-old male NOD.CB17-Prkdc^{scid}/J (NOD/SCID) mice were used as the human-in-mouse xenograft-transplantation model by injecting MOLM14 cells, which were also sensitive to Brusatol and was significantly inhibited in the cell viability and cell cycle assays (FIG. 5A and FIGS. 7E-7F). MOLM14 cells also highly express PI3Kγ but not PI3KC2p (FIG. 7G). Moreover, the MOLM14 mouse model is a straightforward and rapid in vivo assay for preclinical studies. Here, MOLM14 cells were injected subcutaneously, and when xenografts reached 100 mm³ the compounds (Brusatol, 14, 15, 26, 31) or PBS buffer were injected intraperitoneally three times weekly. After two weeks of treatment, the mice were euthanized and tumors were excised (FIG. 7H). The tumor size of the treated mice clearly showed that analogs 15 and 26 exhibited a strong and rapid inhibitory response compared to analogs 14 and 31 tested (FIGS. 8D-8E). The monitored weight of the mice did not show any significant changes (FIG. 7I). Therefore, these results show that these new analogs of Brusatol had significantly comparable bioactivity with the parent Brusatol.

### Example 6: Compounds 26 and 31 Structure-Activity Relationship (SAR)

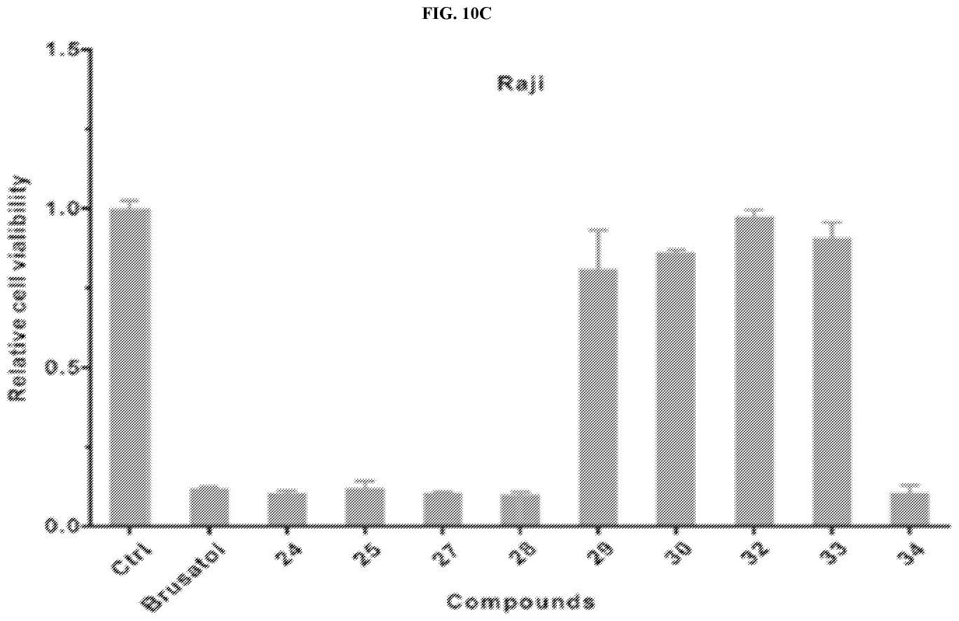

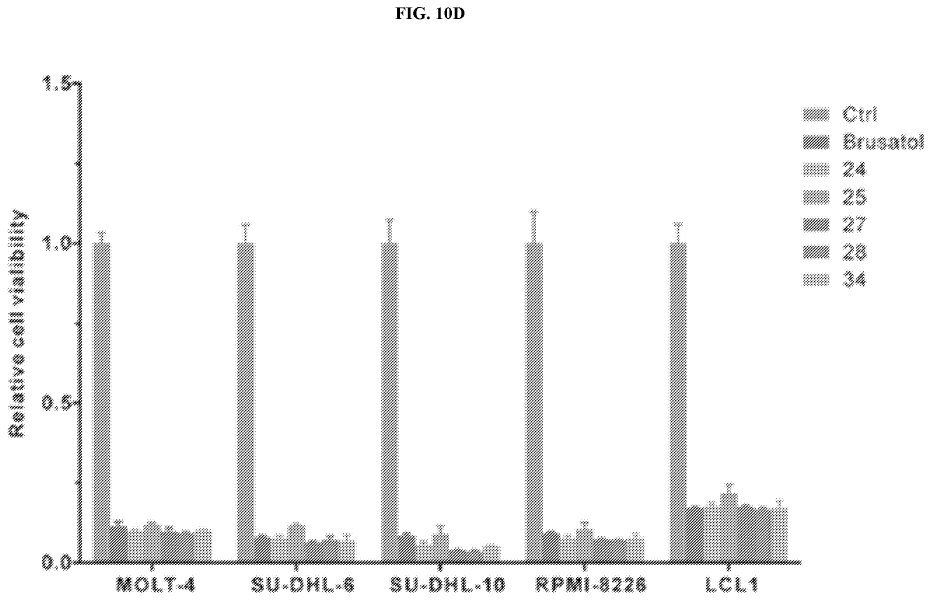

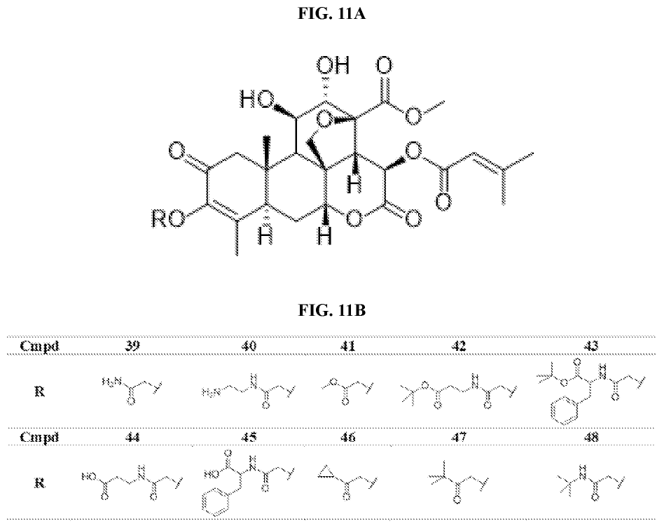

The superior activities of the compounds with homoalanine (26) and proline moieties (31) compared to other amino acids used for conjugation prompted an investigation of the structure-activity relationship around these two amino acids. Therefore, structurally close analogs including D-β-homoalanine and D-proline, derivatives of 26 like acetyl and methylsulfonyl, and 31 were prepared and evaluated for their activities on the Raji cell line (FIGS. 10A-10D). The results showed that compounds 25 and 27, with opposite amino acid configurations, and 28 and 34, with an acetyl on the β-homoalanine and a geminal difluoro substitution on the proline respectively, possessed similar activities. But 29, with a methylsulfonyl group, and 30-33, with side chains more hydrophobic than the methyl in the β-homoalanine, lost activities (FIG. 10C). The active analogs also presented potencies in the five other cell lines comparable to that of the parent brusatol (FIG. 10B).

### Example 7: Stereochemical and Hydrolytic Importance of Conjugated Group

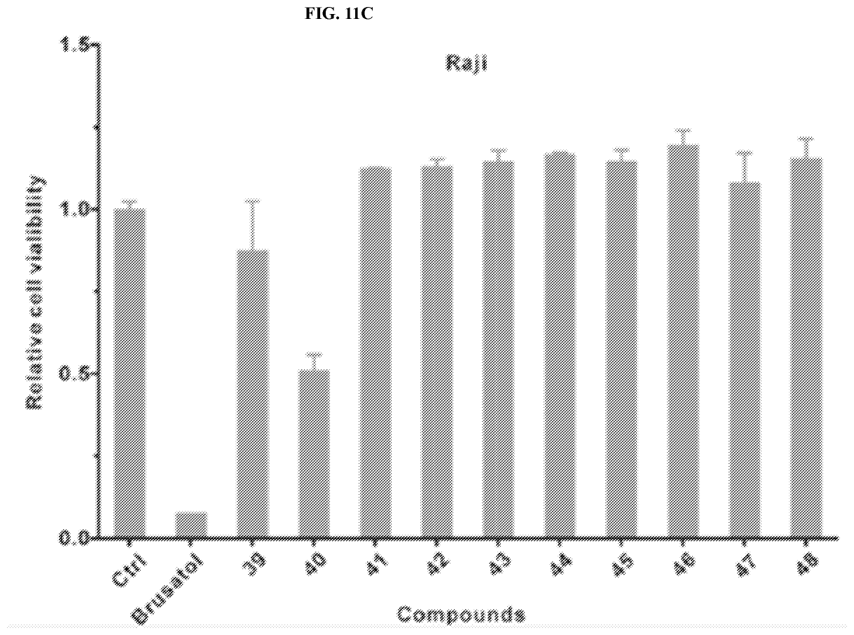

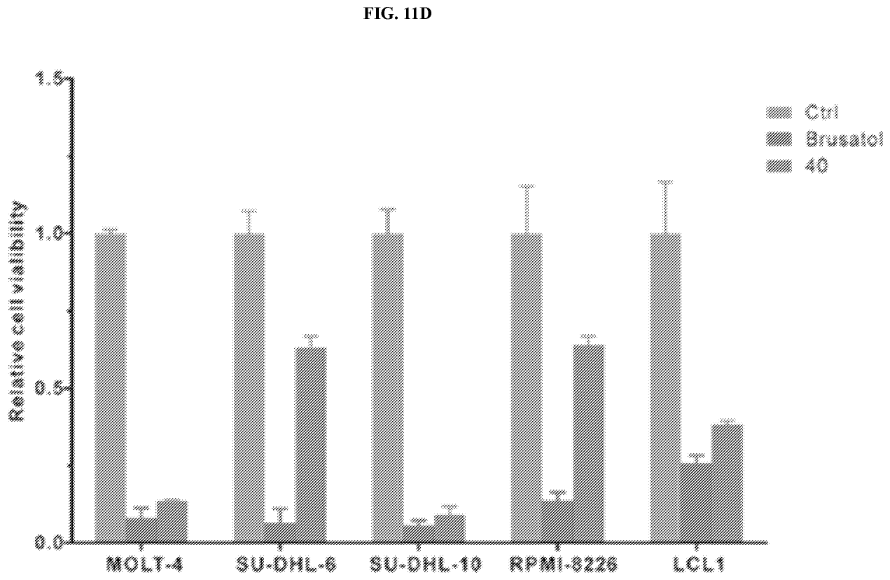

The non-stereoselective feature of β-homoalanine and proline in terms of chirality when conjugated to brusatol (26 vs. 25 and 31 vs. 27) triggered an inquisition as to whether the activities of these conjugates were from brusatol instead of from the conjugates, and if the function of these amino acids or peptides was just being a delivery tool before being cleaved off by esterases. To address this possibility, analogs of 15 and 20 were synthesized (i.e. compounds 39-48) which have polar or hydrophobic terminals connected via a covalent ether bond to brusatol, as opposed to an ester. Due to the non-cleavable nature of these ether bonds under regular conditions, these compounds were not expected to release brusatol by interactions with esterase. In vitro tests revealed that the majority of these analogs were not active, indicating that introduction of these hydrophobic groups, polar amide, and acids had a detrimental effect on the activity of the brusatol pharmacophore. However, a diglycine peptide analog (i.e. compound 40) which bears a free amine group like in 26 and is a close analog of diglycine conjugate 20, demonstrated moderate activity on the Raji cell line (FIGS. 11A-11C) and moderate to good activities on other extended cell lines (FIG. 11D) as well, suggesting that a conjugate itself may have some activity, albeit with reduced potency as compared to the parent brusatol.

### Example 8: Analog Stability

Stability is an important factor to consider for prodrug conjugates. With several brusatol conjugates which have comparable potencies in hand, 15 and 26 were selected from two different families in order to evaluate and to compare to brusatol using in vitro assays that are related to intravenous (IV) and oral (PO) drug administrations (Table 8). Compound 15 has relatively low stability in simulated gastric fluid (SGF) and simulated intestinal fluid (SIF), and thus is not a good candidate for PO administration. Compound 26 has a good solubility in water despite being slightly less soluble than brusatol. Brusatol is stable in both human and rat plasmas.

TABLE 8
solubility and stability evaluation of 26 compared to Brusatol

| | | | | Stability in | Stability in |
|---|---|---|---|---|---|
| | | Plasma stability remaining | | simulated | simulated |
| | Water solubility | % at 60 min | | gastric fluid | intestinal fluid |
| Compound | (μg/mL) | human | rat | (% at 4 h) | (% at 4 h) |
| brusatol | 902 | 91 | 100 | 109 | 100 |
| 26-TFA | 738 | 44 (47)^{a} | 39 (60)^{a} | 97 | 76 |
| 15 | NA | NA | NA | 25 | 42 |

^{a}percentage of brusatol converted from 26

However, a decrease in the amount of 26 and the generation of brusatol were observed. At 60 min, 44% of 26 remained and 47% of UPB-26 was converted into brusatol (Table 8) in human plasma; while only 39% was left and 60% of conversion to brusatol was recorded in rat plasma. These results demonstrated that 26 can be transformed into brusatol in both human and rat plasmas at moderate rates. When these two compounds were tested in both SGF and SIF, high stabilities were observed for brusatol. Compound 26 also displayed good percentage of recovery, with 97% and 76% respectively after 4 h of incubation.

### Example 9: Microsomal Metabolism Changes in Mice Upon Administration of 26

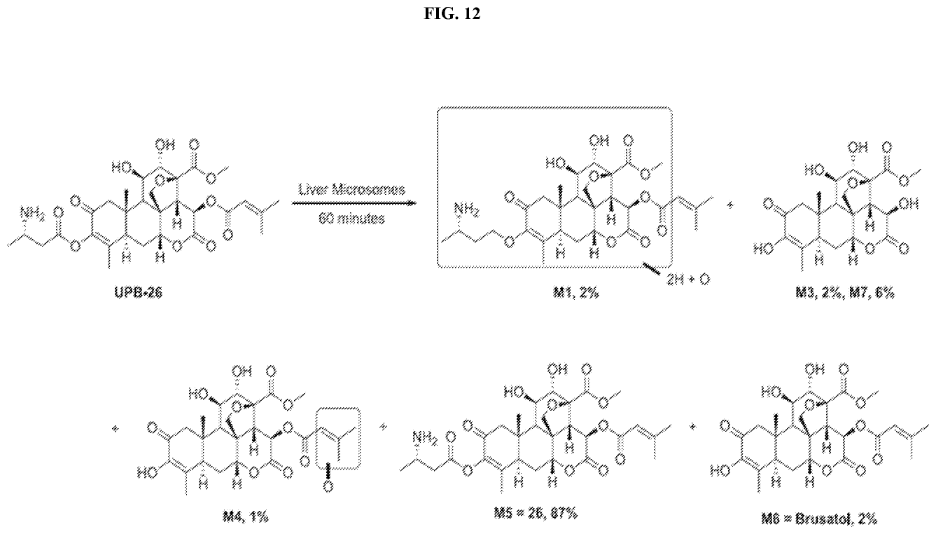

In order to determine if 26 induces liver microsomal metabolism changes, the MetID profile of 26 in mouse liver microsomes was investigated under the same conditions as for brusatol. As illustrated in FIG. 12, a different pattern of metabolism was observed for 26, as compared to that of brusatol. However, 87% of 26 (area %) was detected after 60 min of incubation while five identified metabolites were all in relatively small percentages. In comparison to the performance of the parent compound brusatol (40%) 26 has significantly improved metabolic stability in the mouse liver.

### Example 10: One Novel Brusatol Analog is a Potential PI3K Inhibitor with Minimal Toxicity

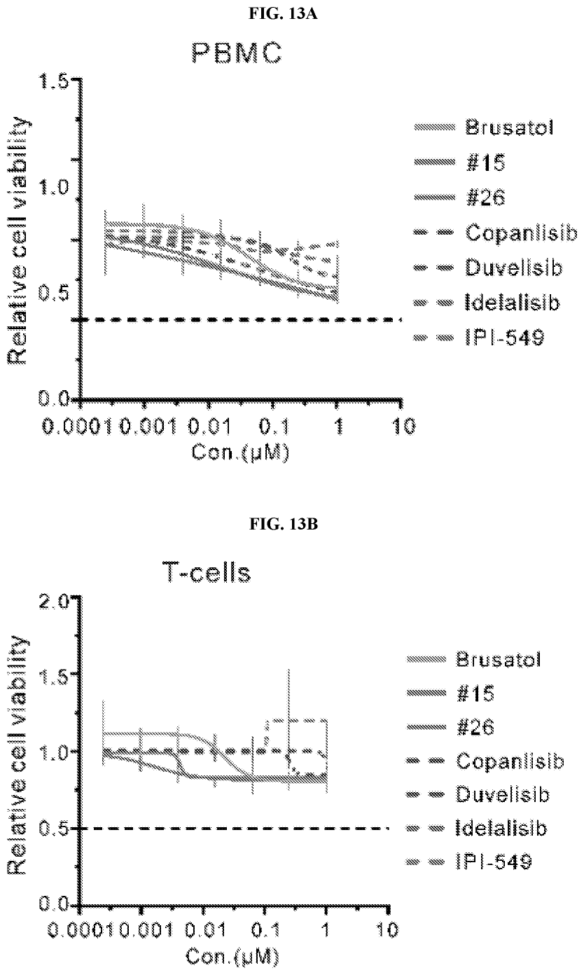

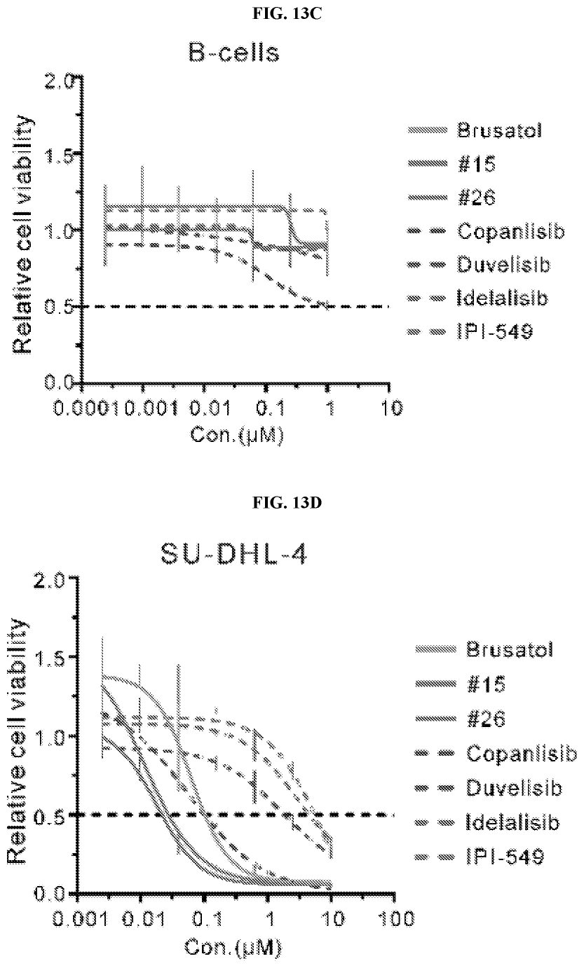

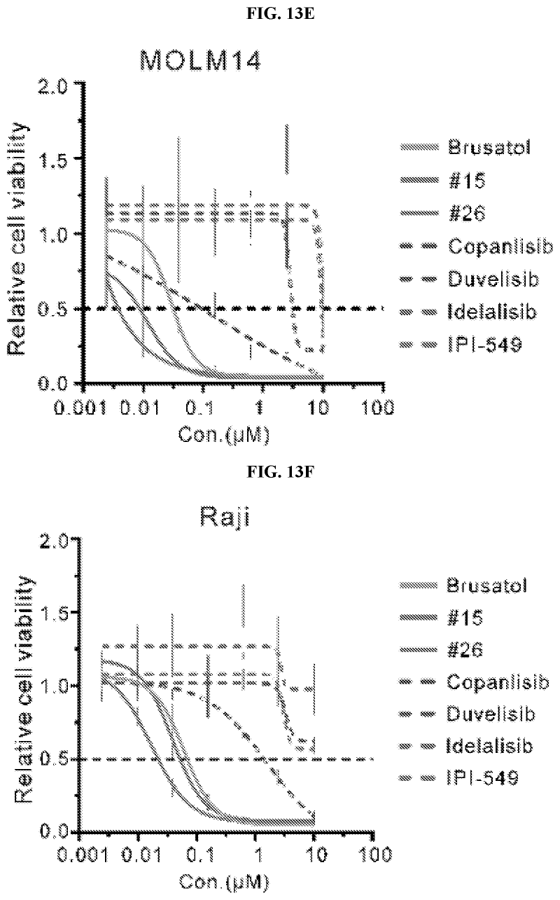

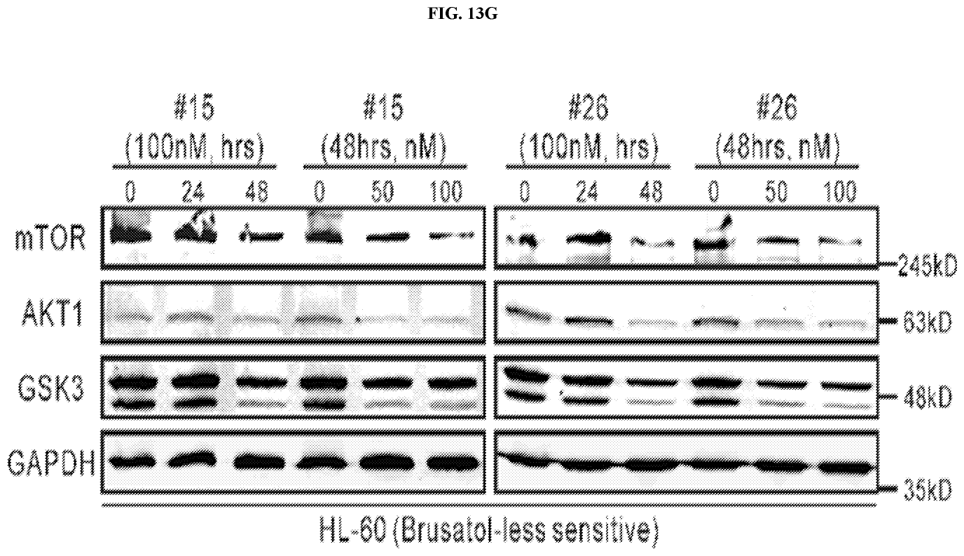

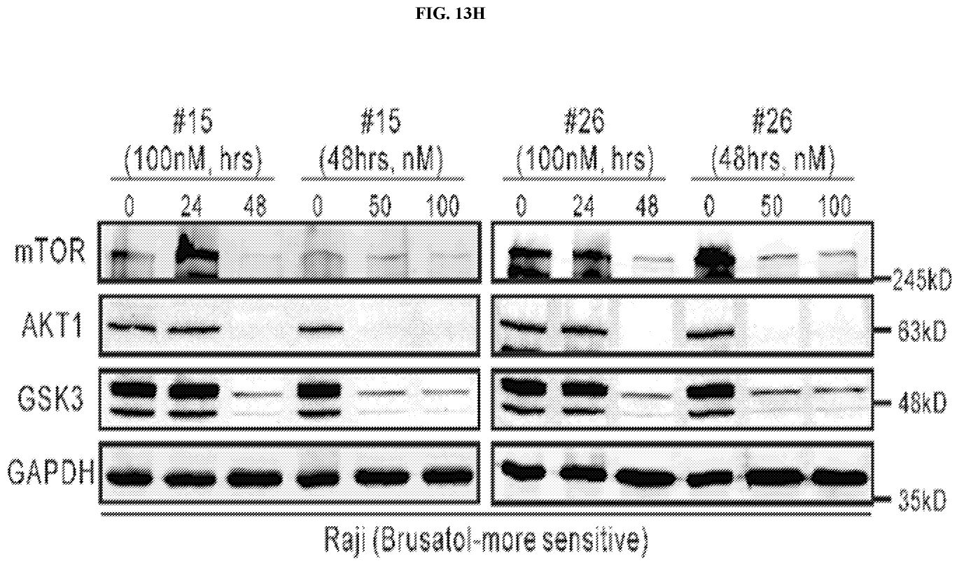

A crucial limitation in the development of Brusatol as a therapeutic agent was linked to its associated toxicities. To examine the potential cytotoxicity of these compounds, human PBMC, T-cells, and B-cells were treated with these compounds (Brusatol, 15, and 26), three FDA approved inhibitors (Copanlisib, Duvelisib, and Idelalisib), and IPI-549 in clinical trials. Importantly, the analogs did not show any significant cytotoxicity in these normal human cell lines when compared with the approved PI3K inhibitors (FIGS. 13A-13C). Furthermore, cell viability assessed in Brusatol-sensitive cells, including SU-DHL-4, MOLM14, and Raji, showed that inhibition exhibited by the two analogs were comparable to the pan-PI3K inhibitor Copanlisib, and had greater efficacy compared to the other PI3K inhibitors (FIG. 13D-13F). These results provide strong evidence that Brusatol and the novel analogs have the potential for clinical use as PI3K inhibitors. It should be noted that overexpressed PI3Kδ in the Brusatol-sensitive cells may also be a potential target as it was also upregulated (FIG. 5D).

To investigate whether the two novel analogs 15 and 26 also specifically targeted the PI3K/AKT signaling pathway, the Brusatol-less sensitive HL-60 cells, and Brusatol-more sensitive Raji cells were treated with these analogs for different periods. Western blot analyses showed that the developed analogs were largely similar to Brusatol in the suppression of AKT1, GSK3, and mTOR levels in Raji, but not HL-60 cells (FIGS. 13G-13H). These findings demonstrated that these analogs also targeted the PI3K/AKT signaling pathway. However, 26 analog inhibited both PI3KCG and GSK3B mRNA expression in vivo, which was different from the inhibitory effects of Brusatol and 15 (FIG. 7J).

The toxicity of these compounds were next tested in vivo. The active compounds (Brusatol, 15, and 26) were intraperitoneally injected with 10 mg/kg every other day. The mice were monitored and euthanized after 24 days of treatment. Brusatol and analog 15 were obviously lethal for the majority of mice within the first 4 days. However, analog 26 did not show any obvious signs of toxicity even after more than three weeks of treatment (FIG. 13I). These results demonstrated that 26 analog has a significantly reduced toxicity compared to Brusatol and analog 15. Therefore, the novel analog (26) had significant inhibitory effects combined with less toxicity, demonstrating strong potential for development as a small molecule therapeutic for the treatment of EBV-positive and other hematologic malignancies.

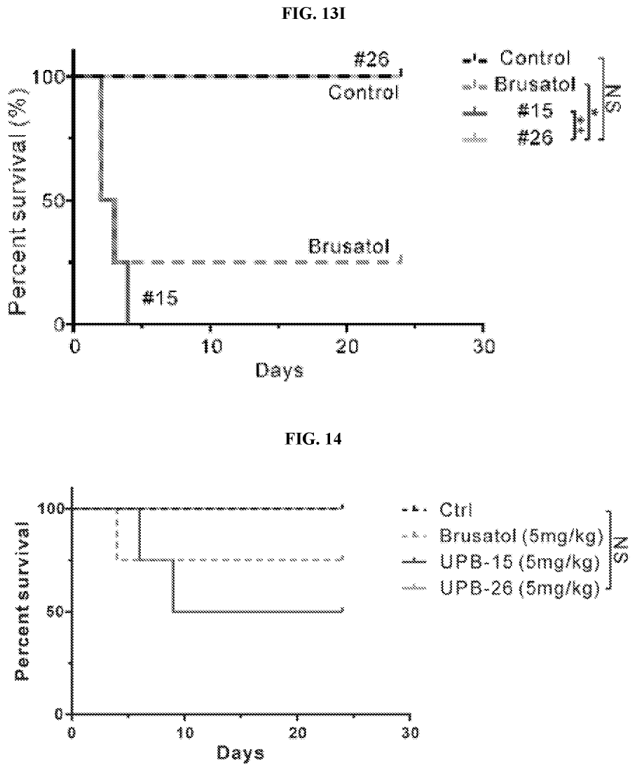

To further assess the toxicity profile of these conjugates of brusatol, the toxicity of 15 and 26 in comparison to brusatol was examined. Six-week-old male NOD.CB17PrkdcSCID/J (NOD/SCID) mice (Jackson Labs, Bar Harbor, Me., USA; 4 mice per group) were intraperitoneally injected with a dose of 5 mg/kg of one of three compounds—brusatol, 15, or 26—every other day, three times per week. The mice were monitored and euthanized after 24 days of treatment. The survival curve, shown in FIG. 14, appears that 26 is less toxic than brusatol. At 5 mg/kg, all four mice survived in the group treated with 26, compared to the three from the group with brusatol treatment and two with 15 treatment. Previously, the toxicities of UPB-15, UPB-26, and brusatol were evaluated at 10 mg/kg. From these two studies it is apparent that there is a dose dependence of brusatol on the toxic effect, with 75% of survival at 5 mg/kg but 25% at 10 mg/kg. Compound 15 appears to be even more toxic with 50% of survival at 5 mg/kg and 0% at 10 mg/kg. However, conjugate 26 did not show any obvious signs of toxicity even after more than three weeks of treatment at both doses. These results indicate that conjugate 26 exhibits much less toxicity than brusatol. Because brusatol is released from conjugate prodrug 26, without wishing to be bound by theory, the reduced toxicity is likely attributed to the improved stability in liver microsomes and moderate rate of conversion to brusatol in plasma.

### Example 11: Pharmacokinetic/Pharmacological Studies

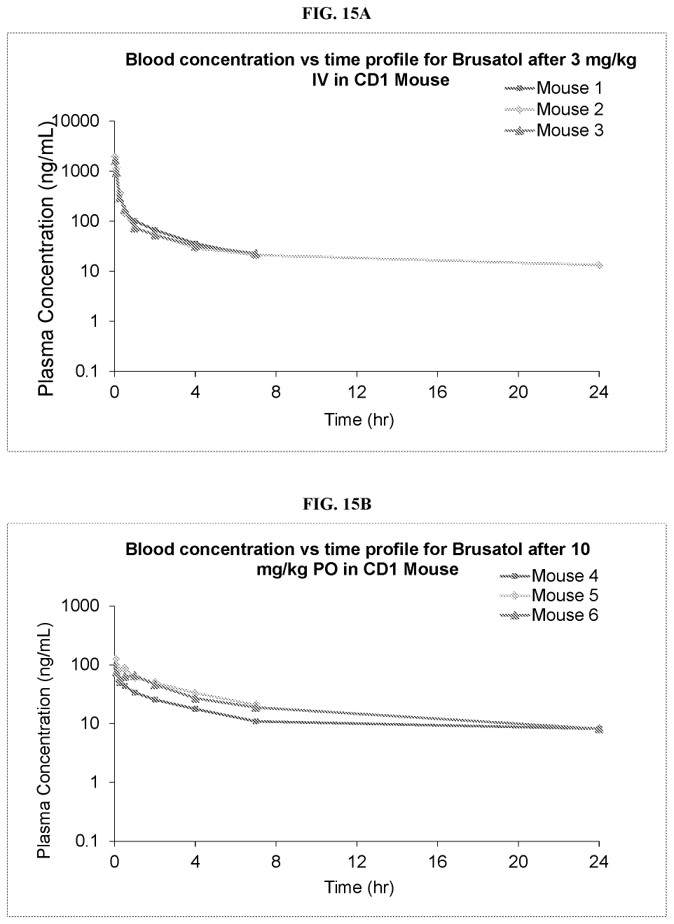

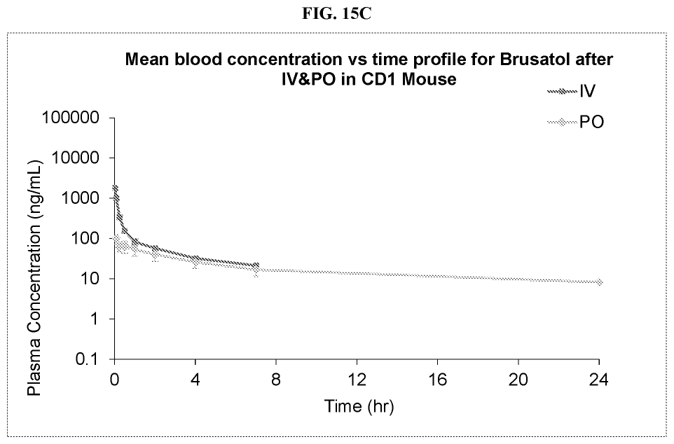

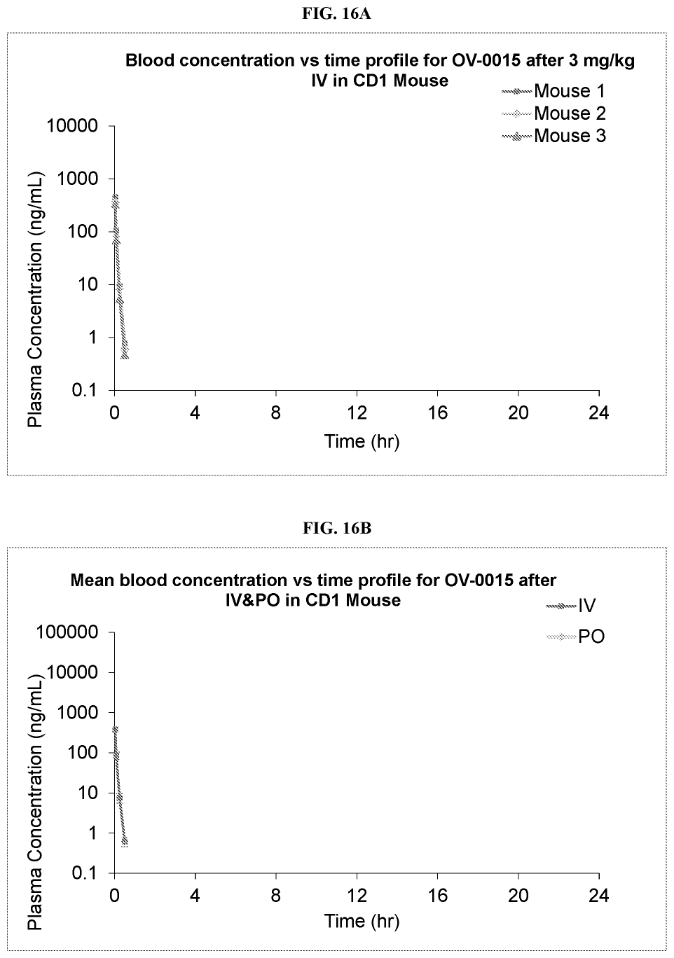

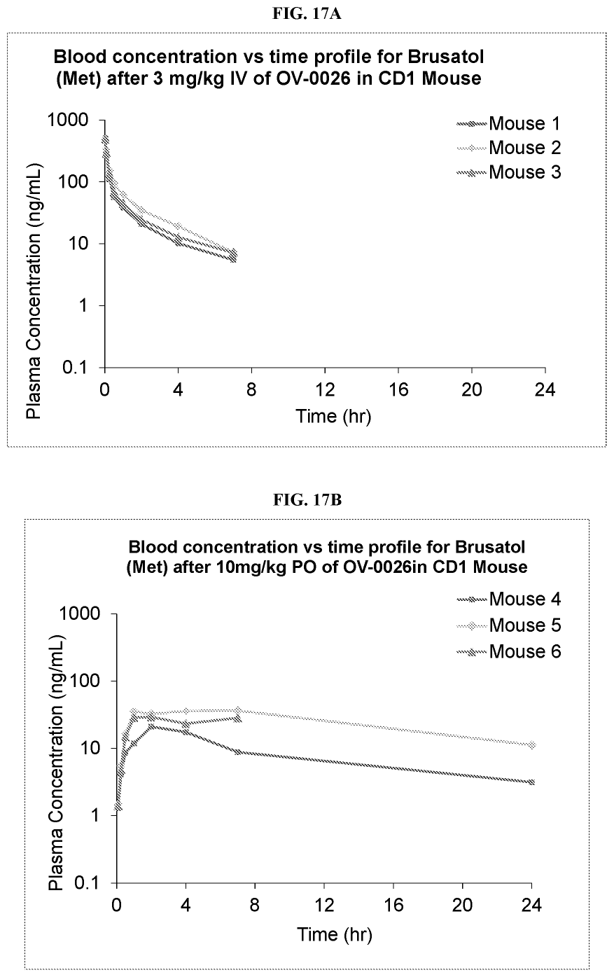

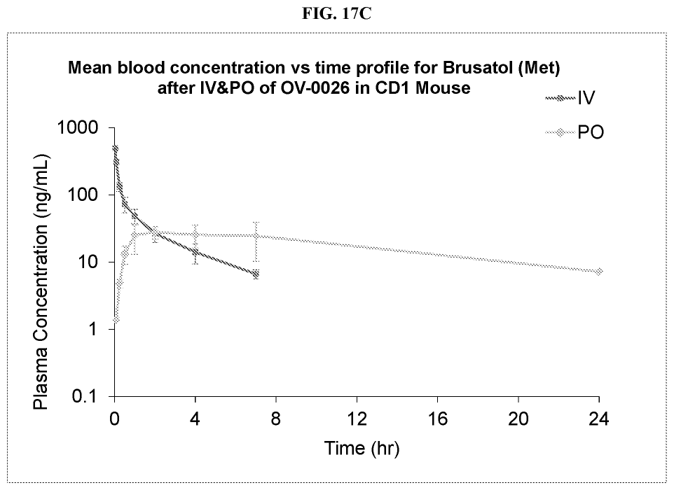

Pharmacokinetic/pharmacological properties of selected compounds of the present disclosure (i.e. compounds 1, 15, and 26) were studied, the results of which are provided herein (FIGS. 15A-15C, FIGS. 16A-16B, and FIGS. 17A-17C).

Compounds were administered to mice intravenously (IV) and orally (PO). Mice were observed for signs of toxicity, including weight loss, general behavior, motor activity, respiratory patterns, and changes in skin. Plasma samples obtained from dosed animals, at ten time points, including 5, 15, and 30 minutes, and 1, 1.5, 2, 4, 6, 8, and 24 h post-dose, were prepared for analysis by means of a single step protein precipitation technique, and analyzed by LC-MS/MS. PK parameters are calculated using established non-compartmental methods.

### Example 12: Synthetic Details

### Synthesis of methyl (1R,2S,3S,3a5,3a1R,4R,6aR,7aR,11aS)-9-(((tert-butoxycarbonyl)glycyl)oxy)-1,2-dihydroxy-8,11a-dimethyl-4-((3-methylbut-2-enoyl)oxy)-5,10-dioxo-1,4,5,6a,7,7a,10,11,11a,11b-decahydro-2H-3,3a1-(epoxymethano)dibenzo[de,g]chromene-3(3aH)-carboxylate (15)

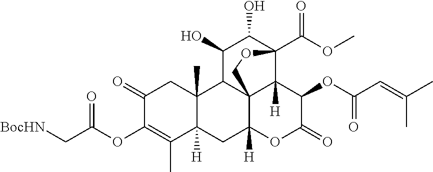

Boc-glycine (25 mg, 0.144 mmol), EDC.HCl (37 mg, 0.192 mmol), and 4-DMAP (12 mg, 0.0962 mmol) was added to brusatol (50 mg, 0.0962 mmol) in 2 mL THF. The reaction was stirred overnight. After diluting with EtOAc, the reaction mixture was washed with saturated aqueous NH4Cl twice and then with brine once. The organic phase was concentrated and purified with HPLC with acetonitrile (0.1% TFA) in water (0.1% TFA) at a gradient of 34-69% in 10 min, to provide the title compound as a white solid (36.4 mg, 55.9%). ¹H NMR (300 MHz, CDCl₃): δ 5.59 (s, 1H), 4.78 (broad s, 1H), 4.69 (d, J=7.5 Hz, 1H), 4.18-4.10 (m, 2H), 4.10-4.04 (m, 2H), 3.75-3.68 (m, 4H), 3.36-3.32 (m, 1H), 3.20-3.08 (m, 1H), 3.06-2.96 (m, 1H), 2.94-2.64 (m, 2H), 2.43-2.28 (m, 2H), 2.13 (s, 3H), 2.08-2.02 (m, 1H), 1.88 (s, 3H), 1.85-1.72 (m, 1H), 1.77 (s, 3H), 1.42 (s, 3H), 1.40 (s, 9H); Calculated for C₃₃H₄₃NO₁₄, 677.69; observed (M+H)+ 678.8.

### Synthesis of methyl (1R,2S,3S,3a5,3a1R,4R,6aR,7aR,11aS)-9-((L-phenylalanyl)oxy)-1,2-dihydroxy-8,11a-dimethyl-4-((3-methylbut-2-enoyl)oxy)-5,10-dioxo-1,4,5,6a,7,7a,10,11,11a,11b-decahydro-2H-3,3a1-(epoxymethano)dibenzo[de,g]chromene-3(3aH)-carboxylate (14)

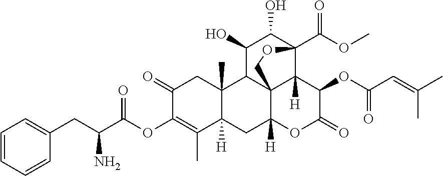

According to the procedure for preparation of compound 15, brusatol (20 mg, 0.0385 mmol) was treated with Boc-L-phenylalanine (15 mg, 0.0577 mmol), EDC.HCl (15 mg, 0.769 mmol), and 4-DMAP (4.7 mg, 0.0385 mmol) afford 10 [HPLC purification with a gradient of 41-76% of acetonitrile (0.1% TFA) in water (0.1% TFA) in 10 min], which was converted to 14 as a clear oil upon acidic deprotection. ¹H NMR (300 MHz, CDCl₃): δ 7.34-7.26 (m, 5H), 5.576 (s, 1H), 4.774 (s, 1H), 4.656 (d, J=7.5 Hz, 1H), 4.48-4.32 (m, 1H), 4.44-4.34 (m, 1H), 4.16-4.06 (m, 2H), 3.70 (s, 3H), 3.62-3.52 (m, 2H), 3.32-3.26 (m, 2H), 3.22-2.95 (m, 1H), 2.95-2.84 (m, 2H), 2.43-2.25 (m, 2H), 2.11 (s, 3H), 2.07-2.00 (m, 1H), 1.86 (s, 3H), 1.75-1.63 (m, 3H), 1.62-1.56 (m, 1H), 1.42-1.16 (m, 3H); Calculated for C₃₅H₄₁NO₁₂, 667.70; observed (M+H)+ 668.8.

### Synthesis of methyl (1R,2S,3S,3a5,3a1R,4R,6aR,7aR,11aS)-9-(((S)-3-aminobutanoyl)oxy)-1,2-dihydroxy-8,11a-dimethyl-4-((3-methylbut-2-enoyl)oxy)-5,10-dioxo-1,4,5,6a,7,7a,10,11,11a,11b-decahydro-2H-3,3a1-(epoxymethano)dibenzo[de,g]chromene-3(3aH)-carboxylate (26)

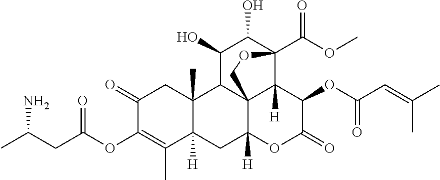

According to the procedure for preparation of compound 15, brusatol (50 mg, 0.0962 mmol) was treated with Boc-β-HoAla-OH (29 mg, 0.144 mmol), EDC.HCl (37 mg, 0.192 mmol), and 4-DMAP (12 mg, 0.0962 mmol) afford 12 [HPLC purification with a gradient of 34-69% of acetonitrile (0.1% TFA) in water (0.1% TFA) in 10 min], which was converted to 26 upon acidic deprotection. There were two isomers in the mixture, which were separated by HPLC with a gradient of 20-55% over 10 min. The title product was obtained in the second fraction as a white solid (20.1 mg, 29.0% in two steps). ¹H NMR (300 MHz, CDCl₃): δ 5.59 (s, 1H), 4.79 (s, 1H), 4.68 (d, J=8.1 Hz, 1H), 4.18-4.10 (m, 2H), 3.76-3.62 (m, 4H), 3.36-3.30 (m, 2H), 3.21-3.11 (m, 1H), 3.09-3.00 (m, 1H), 2.95-2.78 (m, 5H), 2.46-2.28 (m, 2H), 2.13 (s, 3H), 2.10-2.02 (m, 1H), 1.88 (s, 3H), 1.86-1.80 (m, 1H), 1.78 (s, 3H), 1.41 (s, 3H), 1.39 (s, 3H); Calculated for C₃₀H₃₉NO₁₂, 605.63; observed (M+H)+ 606.8.

### Synthesis of (1R,2S,3S,3a5,3a1R,4R,6aR,7aR,11aS)-1,2-dihydroxy-3-(methoxycarbonyl)-8,11a-dimethyl-4-(3-methylbut-2-enoyl)oxy)-5,10-dioxo-1,3,3a,4,5,6a,7,7a,10,11,11a,11b-dodecahydro-2H-3,3a1-(epoxymethano)dibenzo[de,g]chromen-9-yl L-prolinate (31)

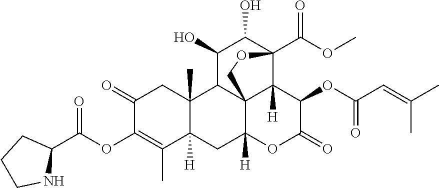

According to the procedure for preparation of compound 15, brusatol (20 mg, 0.0385 mmol) was treated with Boc-Pro-OH (12 mg, 0.0577 mmol), EDCI.HCl (15 mg, 0.769 mmol), and 4-DMAP (4.7 mg, 0.0385 mmol) afford 11 [HPLC purification with a gradient of 35-70% of acetonitrile (0.1% TFA) in water (0.1% TFA) in 10 min], which was converted to 31 as a clear oil (10.7 mg, 38.2%) upon acidic deprotection. ¹H NMR (300 MHz, CDCl₃): δ 5.59 (s, 1H), 4.79 (broad s, 1H), 4.71-4.64 (m, 2H), 4.17-4.09 (m, 2H), 3.75-3.65 (m, 5H), 3.64-3.54 (m, 2H), 3.36-3.30 (m, 2H), 3.20-3.70 (m, 2) 2.58-2.44 (m, 2H), 2.44-2.28 (m, 2H), 2.13 (s, 3H), 2.18-2.02 (m, 3H), 2.00-1.92 (m, 2H), 1.88 (s, 3H), 1.84-1.74 (m, 2H), 1.64-1.40 (m, 3H); Calculated for C₃₁H₃₀NO₁₂, 617.64; observed (M+H)+ 618.8.

### Synthesis of (1R,2S,3S,3aS,3a1R,4R,6aR,7aR,11aS)-1,2,9-trihydroxy-3-((2-methoxyethyl)carbamoyl)-8,11a-dimethyl-5,10-dioxo-1,3,3a,4,5,6a,7,7a,10,11,11a,11b-dodecahydro-2H-3,3a1-(epoxymethano)dibenzo[de,g]chromen-4-yl 3-methylbut-2-enoate (4a)

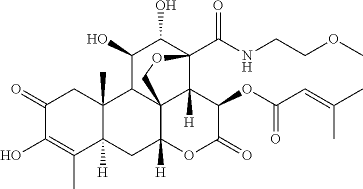

LiOH(aq) (1 M, 1.1 eq) was added to brusatol in THF, and the reaction was stirred overnight. The resulting intermediate was purified by HPLC with acetonitrile (0.1% TFA) in water (0.1% TFA) to yield the corresponding carboxylic acid species. ¹H NMR (CDCl₃-MeOD): 6.33 (broad s, 1H, H¹⁵), 5.68 (s, 1H, H^{2′}), 4.80 (s, 1H, H⁷), 4.74 (d, J=7.6 Hz, 1H, H^{20b}), 4.22 (s, 1H, H¹²), 4.13 (s, 1H, H¹¹), 3.80 (d, J=7.3 Hz, 1H, H¹⁴), 3.09 (d, J=10.3 Hz, 1H, H^{20a}), 2.98 (s, 1H, H^{1b}), 2.93 (s, 1H, H⁵), 2.50-2.30 (m, 2H, H⁹ & H^{1b}), 2.18 (s, 3H), 2.21-2.10 (m, 1H, H^{6a}), 1.91 (s, 3H), 1.85 (s, 3H), 1.84-1.68 (m, 1H, H^{6b}), 1.39 (s, 3H); Calculated for C₂₅H₃₀O₁₁, 506.2; observed 507.6.

To the carboxylic acid species (10 mg, 0.020 mmol) with excess triethylamine in 2 mL DCM, was added EDC.HCl (19 mg, 0.099 mmol), HOBT.H₂O (13 mg, 0.099 mmol), and 2-methoxyethanamine (9 μL, 0.099 mmol). The reaction was stirred at room temperature overnight. Then it was partitioned between EtOAc and saturated aqueous NH₄Cl. The organic phase was concentrated and purified by HPLC, resulting in the product as a white solid. ¹H NMR (300 MHz, CDCl₃): δ 5.59 (s, 1H), 4.78-4.68 (m, 2H), 4.14-4.09 (m, 1H), 3.91 (s, 1H), 3.71 (d, J=7.5 Hz, 1H), 3.42-3.36 (m, 2H), 3.36-3.31 (m, 3H), 3.31-3.28 (m, 2H), 2.72-2.61 (m, 2H), 2.39-2.25 (m, 2H), 2.12 (s, 3H), 2.10-2.07 (m, 1H), 1.86 (s, 3H), 1.81-1.76 (m, 3H), 1.75-1.63 (m, 1H), 1.32 (s, 3H). Calculated for C₂₈H₃₇NO₁₁, 563.59; observed (M+H)+ 564.7.

### Synthesis of (1R,2S,3S,3a5,3a1R,4R,6aR,7aR,11aS)-1,2,9-trihydroxy-3-(isopentylcarbamoyl)-8,11a-dimethyl-5,10-dioxo-1,3,3a,4,5,6a,7,7a,10,11,11a,11b-dodecahydro-2H-3,3a1-(epoxymethano)dibenzo[de,g]chromen-4-yl 3-methylbut-2-enoate (4b)

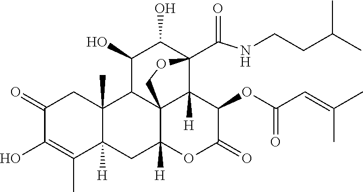

According to the procedure for preparation of compound 4a, 3 (10 mg, 0.020 mmol) was treated with EDC.HCl (19 mg, 0.099 mmol), HOBT.H₂O (13 mg, 0.099 mmol), and isoamylamine (11 μL, 0.099 mmol) to afford 4b as a white solid. ¹H NMR (300 MHz, CDCl₃): δ 5.64 (s, 1H), 4.79 (d, J=7.5 Hz, 1H), 4.73 (broad s, 1H), 4.19-4.15 (m, 1H), 3.93 (broad s, 1H), 3.71 (d, J=7.5 Hz, 1H), 3.46-3.36 (m, 1H), 3.36-3.22 (m, 1H), 3.18-3.04 (m, 1H), 2.98-2.86 (m, 2H), 2.42-2.28 (m, 2H), 2.25-2.10 (m, 3H), 1.91-1.86 (m, 4H), 1.75-1.65 (m, 3H), 1.64-1.50 (m, 2H), 1.41-1.36 (m, 2H), 1.35 (s, 3H), 0.92-0.85 (m, 7H)); Calculated for C₃₀H₄₁NO₁₀, 575.65; observed (M+H)+ 576.8.

### Synthesis of methyl (1R,2S,3S,3a5,3a1R,4R,6aR,7aR,11aS)-1,2,9-trihydroxy-8,11a-dimethyl-4-((3-methylbut-2-enoyl)oxy)-5,10-dioxo-1,4,5,6a, 7,7a,10,11,11a,11b-decahydro-2H-3,3a1-(epoxymethano)dibenzo[de,g]chromene-3(3aH)-carboxylate (1, Brusatol)

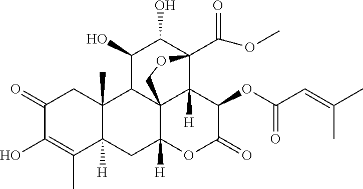

To 3 (15 mg, 0.030 mmol) in 2 mL MeOH, EDC.HCl (11 mg, 0.059 mmol) and 4-DMAP (4 mg, 0.030 mmol) was added. The reaction was stirred at room temperature overnight. Then it was partitioned between EtOAc and saturated aqueous NH₄Cl. The organic phase was concentrated down and purified by HPLC, resulting in the product as a white solid (4.9 mg, 32%). ¹H NMR (300 MHz, methanol-d₄): δ 5.67 (s, 1H), 4.83 (broad s, 1H), 4.70 (d, J=7.6 Hz, 1H), 4.22-4.14 (m, 2H), 3.74-3.68 (m, 4H), 3.40-3.20 (m, 2H), 3.01-2.92 (m, 1H), 2.88-2.79 (m, 1H), 2.57-2.48 (m, 1H), 2.35-2.25 (m, 1H), 2.23-2.19 (m, 1H), 2.17 (s, 3H), 1.94 (s, 3H), 1.89-1.85 (m, 1H), 1.84 (s, 3H), 1.37 (s, 3H); Calculated for C₂₅H₃₀O₁₁, 520.53; observed (M+H)+ 521.7.

### Synthesis of isopropyl (1R,2S,3S,3aS,3a1R,4R,6aR,7aR,11aS)-1,2,9-trihydroxy-8,11a-dimethyl-4-((3-methylbut-2-enoyl)oxy)-5,10-dioxo-1,4,5,6a,7,7a,10,11,11a,11b-decahydro-2H-3,3a1-(epoxymethano)dibenzo[de,g]chromene-3(3aH)-carboxylate (5)

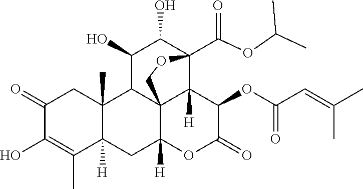

According to the procedure for preparation of compound 1, compound 3 (15 mg, 0.030 mmol) was treated with EDC.HCl (11 mg, 0.059 mmol) and 4-DMAP (4 mg, 0.030 mmol), and isopropanol (2 mL) to afford 5 as a white solid (3.5 mg, 21%). ¹H NMR (300 MHz, methanol-d₄): δ 5.67 (s, 1H), 4.83-4.79 (m, 1H), 4.69 (d, J=7.6 Hz, 1H), 4.21-4.14 (m, 2H), 3.75-3.66 (m, 1H), 3.40-2.20 (m, 3H), 3.02-2.92 (m, 1H), 2.88-2.80 (m, 1H), 2.58-2.48 (m, 1H), 2.35-2.25 (m, 1H), 2.25-2.19 (m, 1H), 2.16 (s, 3H), 2.00-1.89 (m, 4H), 1.89-1.80 (m, 3H), 1.37 (s, 3H), 1.33-1.22 (m, 6H); Calculated for C₂₈H₃₆O₁₁, 548.58; observed (M+H)+ 549.7.

### Synthesis of methyl (1R,2S,3S,3a5,3a1R,4R,6aR,7aR,11aS)-1,2-dihydroxy-8,11a-dimethyl-4-((3-methylbut-2-enoyl)oxy)-5,10-dioxo-9-((5-((3aS,4S,6aR)-2-oxohexahydro-1H-thieno[3,4-d]imidazol-4-yl)pentanoyl)oxy)-1,4,5,6a,7,7a,10,11,11a,11b-decahydro-2H-3,3a1-(epoxymethano)dibenzo[de,g]chromene-3(3aH)-carboxylate (51048)

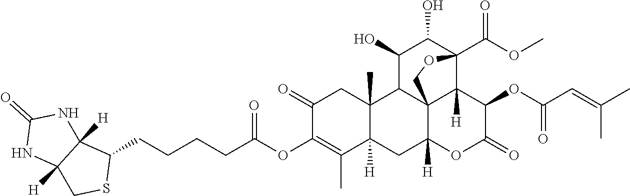

To brusatol (20 mg, 0.0385 mmol) in 2 mL DCM, EDC.HCl (15 mg, 0.769 mmol), and 4-DMAP (4.7 mg, 0.0385 mmol), was added D-biotin (9 mg, 0.0385 mmol). The reaction was stirred at room temperature for 2 days. Then it was diluted with EtOAc and washed with saturated aqueous NH₄Cl and brine. The organic phase was concentrated down and then purified by HPLC with acetonitrile (0.1% TFA) in water (0.1% TFA) at a gradient of 20-60% in 12 min, resulting in the product as a white solid (13.7 mg, 47.7%). ¹H NMR (CDCl₃-methanol-d₄): (ppm) 5.62 (s, 1H), 4.78 (s, 1H), 4.75-4.60 (m, 1H), 4.55-4.35 (m, 2H), 4.34-4.03 (m, 2H), 3.79 (s, 3H), 3.85-3.70 (m, 1H), 3.30-3.00 (m, 3H), 3.00-2.75 (m, 2H), 2.70-2.55 (m, 1H), 2.45-2.00 (m, 5H), 1.98-1.30 (m, 18H). Calculated for C₃₆H₄₆N₂O₁₃S, 746.3; observed 747.4.

### Synthesis of methyl (1R,2S,3S,3aS,3a1R,4R,6aR,7aR,11aS)-1,2-dihydroxy-8,11a-dimethyl-4-((3-methylbut-2-enoyl)oxy)-5,10-dioxo-9-((6-(5-((3aS,4S,6aR)-2-oxohexahydro-1H-thieno[3,4-d]imidazol-4-yl)pentanamido)hexanoyl)oxy)-1,4,5,6a,7,7a,10,11,11a,11b-decahydro-2H-3,3a1-(epoxymethano)dibenzo[de,g]chromene-3(3aH)-carboxylate (51052)

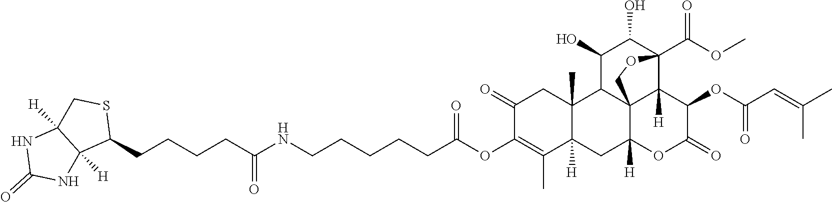

Brusatol (10.2 mg, 0.02 mmol), N-(+)-Biotinyl-6-aminohexanoic acid (7.0 mg, 0.02 mmol), EDCI (7.7 mg, 0.04 mmol), DMAP (2.4 mg, 0.04 mmol) were dissolved in DCM (1 mL). The mixture was stirred under argon at room temperature for a week. Solvent was removed in vacuo, and the residue was partitioned between ethyl acetate and brine. The organic phase was concentrated. The desired conjugate (1.7 mg) was obtained as a white solid after HPLC separation (gradient: 20-53% of acetonitrile:water over 15 minutes) and lyophilization. ¹H NMR (CDCl₃-methanol-d₄): (ppm) 5.69-5.63 (m, 1H, 2′), 4.86-4.81 (m, 1H, 7), 4.80-4.72 (m, 1H, H12), 4.58-4.48 (m, 1H) 4.34-4.27 (m, 2H), 4.27-4.22 (m, 1H), 3.78 (s, 3H), 3.82-3.72 (m, 1H), 3.30-3.10 (m, 4H), 3.03-2.75 (m, 3H), 2.65-2.35 (m, 2H), 2.25-2.15 (m, 7H), 1.90 (s, 3H), 1.90-1.30 (m, 21H); Calculated for C₄₂H₅₇N₃O₁₄S, 859.4; observed 861.0.

### Synthesis of (1R,2S,3S,3a5,3a1R,4R,6aR,7aR,11aS)-1,2,9-trihydroxy-8,11a-dimethyl-5,10-dioxo-3-((6-(4-((3aS,4S,6aR)-2-oxohexahydro-1H-thieno[3,4-d]imidazol-4-yl)butanamido)hexyl)carbamoyl)-1,3,3a,4,5,6a,7,7a,10,11,11a,11b-dodecahydro-2H-3,3a1-(epoxymethano)dibenzo[de,g]chromen-4-yl 3-methylbut-2-enoate (51046)

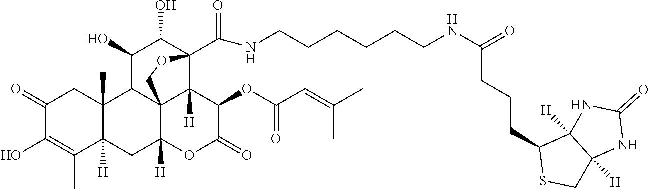

To compound 3 (12 mg, 0.024 mmol) and excess triethylamine in 2 mL DCM, EDC.HCl (23 mg, 0.12 mmol), HOBT.H₂O (16 mg, 0.12 mmol), and N-(6-aminohexyl)-biotinamide (41 mg, 0.12 mmol) was added. The reaction was stirred at room temperature for 2 days. Then it was partitioned between EtOAc and saturated aqueous NH₄Cl. The organic phase was concentrated and then purified by HPLC at a gradient of 20-70% in 20 min to provide a white solid (3.7 mg, %) after lyophilization. ¹H NMR (CDCl₃-methanol-d₄): 7.50-7.38 (m, 1H), 5.64 (s, 1H), 4.90-4.75 (m, 2H), 4.55-4.45 (m, 1H), 4.38-4.25 (m, 1H), 4.23-4.15 (m, 1H), 3.96 (s, 1H), 3.85-3.75 (m, 1H), 3.60-3.30 (m, 1H), 3.30-3.10 (m, 6H), 3.00-2.85 (m, 3H), 2.83-2.68 (m, 1H), 2.50-2.30 (m, 2H), 2.24-2.10 (m, 6H,), 1.93 (s, 3H), 1.85 (s, 3H), 1.85-1.20 (m, 18H).

Compounds 3, 6-13, 16-25, 27-30, 32-34, and 39-59 can be prepared according to the synthetic routes described elsewhere herein and/or methods known to those skilled in the art in view of the teachings provided herein.

### Example 13: Compounds of the Present Disclosure

TABLE 9
Compounds of the present disclosure

| Cmpd | Structure |
|---|---|
| 1 (Brusatol) | 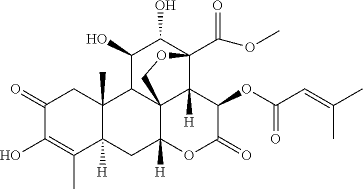 |
| | methyl (1R,2S,3S,3aS,3a1R,4R,6aR,7aR,11aS)-1,2,9-trihydroxy-8,11a-dimethyl- |
| | 4-((3-methylbut-2-enoyl)oxy)-5,10-dioxo-1,4,5,6a,7,7a,10,11,11a,11b- |
| | decahydro-2H-3,3a1-(epoxymethano)dibenzo[de,g]chromene-3(3aH)-carboxylate |
| 3 | 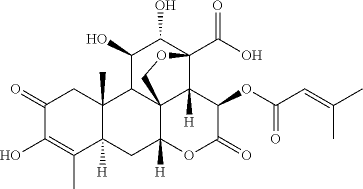 |
| | (1R,2S,3S,3aS,3a1R,4R,6aR,7aR,11aS)-1,2,9-trihydroxy-8,11a-dimethyl-4-((3- |
| | methylbut-2-enoyl)oxy)-5,10-dioxo-1,4,5,6a,7,7a,10,11,11a,11b-decahydro-2H- |
| | 3,3a1-(epoxymethano)dibenzo[de,g]chromene-3(3aH)-carboxylic acid |
| 4a | 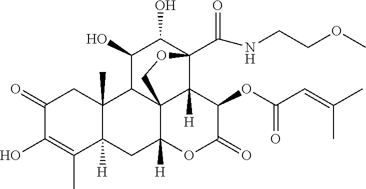 |
| | (1R,2S,3S,3aS,3a1R,4R,6aR,7aR,11aS)-1,2,9-trihydroxy-3-((2- |
| | methoxyethyl)carbamoyl)-8,11a-dimethyl-5,10-dioxo- |
| | 1,3,3a,4,5,6a,7,7a,10,11,11a,11b-dodecahydro-2H-3,3a1- |
| | (epoxymethano)dibenzo[de,g]chromen-4-yl 3-methylbut-2-enoate |
| 4b | 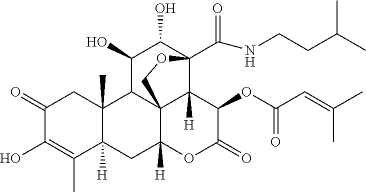 |
| | (1R,2S,3S,3aS,3a1R,4R,6aR,7aR,11aS)-1,2,9-trihydroxy-3- |
| | (isopentylcarbamoyl)-8,11a-dimethyl-5,10-dioxo- |
| | 1,3,3 a,4,5,6a,7,7a,10,11,11a,11b-dodecahydro-2H-3,3a1- |
| | (epoxymethano)dibenzo[de,g]chromen-4-yl 3-methylbut-2-enoate |
| 5 | 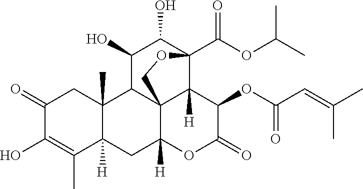 |
| | isopropyl (1R,2S,3S,3aS,3a1R,4R,6aR,7aR,11aS)-1,2,9-trihydroxy-8,11a- |
| | dimethyl-4-((3-methylbut-2-enoyl)oxy)-5,10-dioxo-1,4,5,6a,7,7a,10,11,11a,11b- |
| | decahydro-2H-3,3a1-(epoxymethano)dibenzo[de,g]chromene-3(3aH)-carboxylate |
| 6 | 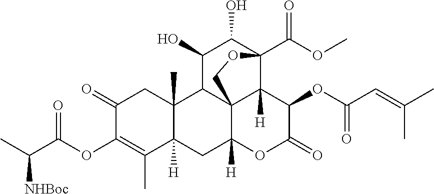 |
| | methyl (1R,2S,3S,3aS,3a1R,4R,6aR,7aR,11aS)-9-((L-alanyl)oxy)-1,2-dihydroxy- |
| | 8,11a-dimethyl-4-((3-methylbut-2-enoyl)oxy)-5,10-dioxo- |
| | 1,4,5,6a,7,7a,10,11,11a,11b-decahydro-2H-3,3a1- |
| | (epoxymethano)dibenzo[de,g]chromene-3(3aH)-carboxylate |
| 7 | 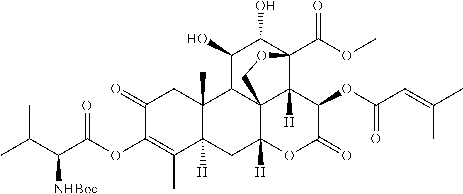 |
| | methyl (1R,2S,3S,3aS,3a1R,4R,6aR,7aR,11aS)-9-(((tert-butoxycarbonyl)-L- |
| | valyl)oxy)-1,2-dihydroxy-8,11a-dimethyl-4-((3-methylbut-2-enoyl)oxy)-5,10- |
| | dioxo-1,4,5,6a,7,7a,10,11,11a,11b-decahydro-2H-3,3a1- |
| | (epoxymethano)dibenzo[de,g]chromene-3(3aH)-carboxylate |
| 8 | 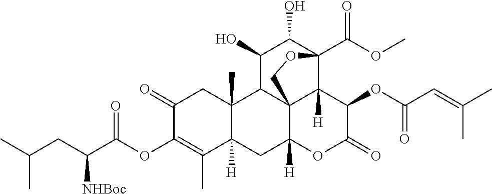 |
| | methyl (1R,2S,3S,3aS,3a1R,4R,6aR,7aR,11aS)-9-(((tert-butoxycarbonyl)-L- |
| | leucyl)oxy)-1,2-dihydroxy-8,11a-dimethyl-4-((3-methylbut-2-enoyl)oxy)-5,10- |
| | dioxo-1,4,5,6a,7,7a,10,11,11a,11b-decahydro-2H-3,3a1- |
| | (epoxymethano)dibenzo[de,g]chromene-3(3aH)-carboxylate |
| 9 | 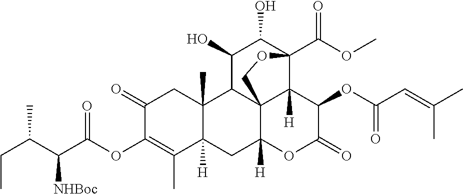 |
| | methyl (1R,2S,3S,3aS,3a1R,4R,6aR,7aR,11aS)-9-(((tert-butoxycarbonyl)-L- |
| | isoleucyl)oxy)-1,2-dihydroxy-8,11a-dimethyl-4-((3-methylbut-2-enoyl)oxy)- |
| | 5,10-dioxo-1,4,5,6a,7,7a,10,11,11a,11b-decahydro-2H-3,3a1- |
| | (epoxymethano)dibenzo[de,g]chromene-3(3aH)-carboxylate |
| 10 | 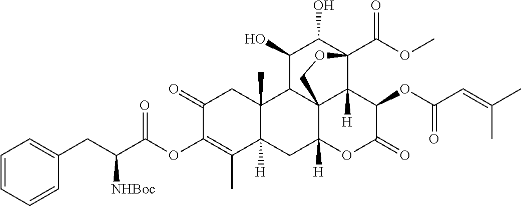 |
| | methyl (1R,2S,3S,3aS,3a1R,4R,6aR,7aR,11aS)-9-(((tert-butoxycarbonyl)-L- |
| | phenylalanyl)oxy)-1,2-dihydroxy-8,11a-dimethyl-4-((3-methylbut-2-enoyl)oxy)- |
| | 5,10-dioxo-1,4,5,6a,7,7a,10,11,11a,11b-decahydro-2H-3,3a1- |
| | (epoxymethano)dibenzo[de,g]chromene-3(3aH)-carboxylate |
| 11 | 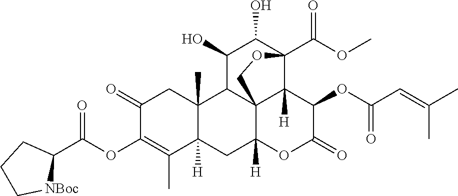 |
| | 1-(tert-butyl) 2-((1R,2S,3S,3aS,3a1R,4R,6aR,7aR,11aS)-1,2-dihydroxy-3- |
| | (methoxycarbonyl)-8,11a-dimethyl-4-((3-methylbut-2-enoyl)oxy)-5,10-dioxo- |
| | 1,3,3a,4,5,6a,7,7a,10,11,11a,11b-dodecahydro-2H-3,3a1- |
| | (epoxymethano)dibenzo[de,g]chromen-9-yl)(2S)-pyrrolidine-1,2-dicarboxylate |
| 12 | 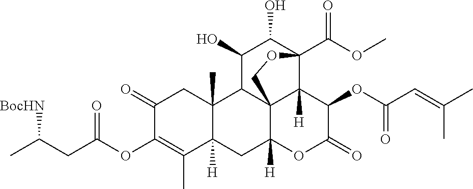 |
| | methyl (1R,2S,3S,3aS,3a1R,4R,6aR,7aR,11aS)-9-(((S)-3-((tert- |
| | butoxycarbonyl)amino)butanoyl)oxy)-1,2-dihydroxy-8,11a-dimethyl-4-((3- |
| | methylbut-2-enoyl)oxy)-5,10-dioxo-1,4,5,6a,7,7a,10,11,11a,11b-decahydro-2H- |
| | 3,3a1-(epoxymethano)dibenzo[de,g]chromene-3(3aH)-carboxylate |
| 13 | 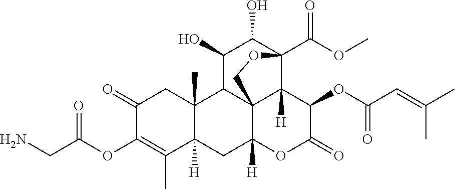 |
| | methyl (1R,2S,3S,3aS,3alR,4R,6aR,7aR,11aS)-9-(glycyloxy)-1,2-dihydroxy- |
| | 8,11a-dimethyl-4-((3-methylbut-2-enoyl)oxy)-5,10-dioxo- |
| | 1,4,5,6a,7,7a,10,11,11a,11b-decahydro-2H-3,3a1- |
| | (epoxymethano)dibenzo[de,g]chromene-3(3aH)-carboxylate |
| 14 | 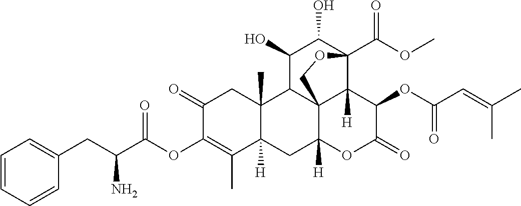 |
| | methyl (1R,2S,3S,3aS,3a1R,4R,6aR,7aR,11aS)-9-((L-phenylalanyl)oxy)-1,2- |
| | dihydroxy-8,11a-dimethyl-4-((3-methylbut-2-enoyl)oxy)-5,10-dioxo- |
| | 1,4,5,6a,7,7a,10,11,11a,11b-decahydro-2H-3,3a1- |
| | (epoxymethano)dibenzo[de,g]chromene-3(3aH)-carboxylate |
| 15 | 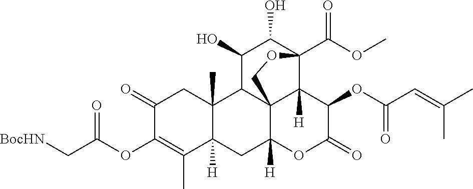 |
| | methyl (1R,2S,3S,3aS,3a1R,4R,6aR,7aR,11aS)-9-(((tert- |
| | butoxycarbonyl)glycyl)oxy)-1,2-dihydroxy-8,11a-dimethyl-4-((3-methylbut-2- |
| | enoyl)oxy)-5,10-dioxo-1,4,5,6a,7,7a,10,11,11a,11b-decahydro-2H-3,3a1- |
| | (epoxymethano)dibenzo[de,g]chromene-3(3aH)-carboxylate |
| 16 | 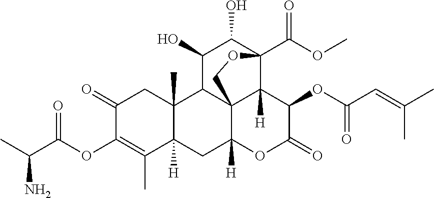 |
| | methyl (1R,2S,3S,3aS,3a1R,4R,6aR,7aR,11aS)-9-((L-alanyl)oxy)-1,2-dihydroxy- |
| | 8,11a-dimethyl-4-((3-methylbut-2-enoyl)oxy)-5,10-dioxo- |
| | 1,4,5,6a,7,7a,10,11,11a,11b-decahydro-2H-3,3a1- |
| | (epoxymethano)dibenzo[de,g]chromene-3(3aH)-carboxylate |
| 17 | 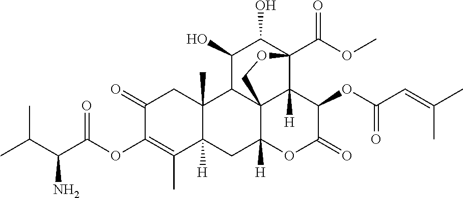 |
| | methyl (1R,2S,3S,3aS,3a1R,4R,6aR,7aR,11aS)-9-((L-valyl)oxy)-1,2-dihydroxy- |
| | 8,11a-dimethyl-4-((3-methylbut-2-enoyl)oxy)-5,10-dioxo- |
| | 1,4,5,6a,7,7a,10,11,11a,11b-decahydro-2H-3,3a1- |
| | (epoxymethano)dibenzo[de,g]chromene-3(3aH)-carboxylate |
| 18 | 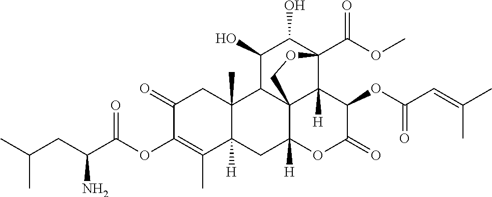 |
| | methyl (1R,2S,3S,3aS,3a1R,4R,6aR,7aR,11aS)-9-((L-leucyl)oxy)-1,2-dihydroxy- |
| | 8,11a-dimethyl-4-((3-methylbut-2-enoyl)oxy)-5,10-dioxo- |
| | 1,4,5,6a,7,7a,10,11,11a,11b-decahydro-2H-3,3a1- |
| | (epoxymethano)dibenzo[de,g]chromene-3(3aH)-carboxylate |
| 19 | 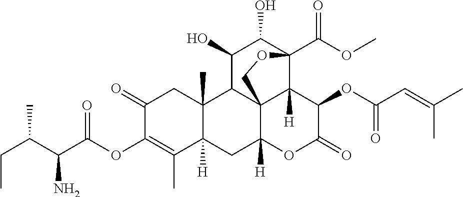 |
| | methyl (1R,2S,3S,3aS,3a1R,4R,6aR,7aR,11aS)-9-((L-isoleucyl)oxy)-1,2- |
| | dihydroxy-8,11a-dimethyl-4-((3-methylbut-2-enoyl)oxy)-5,10-dioxo- |
| | 1,4,5,6a,7,7a,10,11,11a,11b-decahydro-2H-3,3a1- |
| | (epoxymethano)dibenzo[de,g]chromene-3(3aH)-carboxylate |
| 20 | 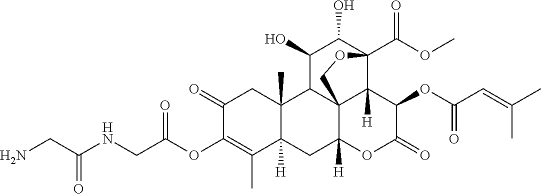 |
| | methyl (1R,2S,3S,3aS,3a1R,4R,6aR,7aR,11aS)-9-((glycylglycyl)oxy)-1,2- |
| | dihydroxy-8,11a-dimethyl-4-((3-methylbut-2-enoyl)oxy)-5,10-dioxo- |
| | 1,4,5,6a,7,7a,10,11,11a,11b-decahydro-2H-3,3a1- |
| | (epoxymethano)dibenzo[de,g]chromene-3(3aH)-carboxylate |
| 21 | 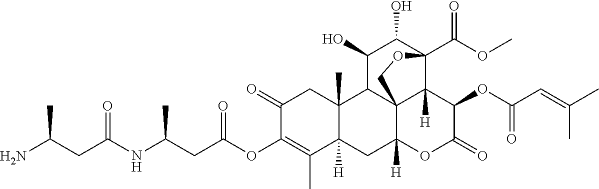 |
| | methyl (1R,2S,3S,3aS,3a1R,4R,6aR,7aR,11aS)-9-(((S)-3-((S)-3- |
| | aminobutanamido)butanoyl)oxy)-1,2-dihydroxy-8,11a-dimethyl-4-((3-methylbut- |
| | 2-enoyl)oxy)-5,10-dioxo-1,4,5,6a,7,7a,10,11,11a,11b-decahydro-2H-3,3a1- |
| | (epoxymethano)dibenzo[de,g]chromene-3(3aH)-carboxylate |
| 22 | 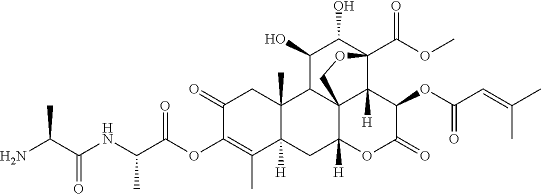 |
| | methyl (1R,2S,3S,3aS,3a1R,4R,6aR,7aR,11aS)-9-((L-alanyl-L-alanyl)oxy)-1,2- |
| | dihydroxy-8,11a-dimethyl-4-((3-methylbut-2-enoyl)oxy)-5,10-dioxo- |
| | 1,4,5,6a,7,7a,10,11,11a,11b-decahydro-2H-3,3a1- |
| | (epoxymethano)dibenzo[de,g]chromene-3(3aH)-carboxylate |
| 23 | 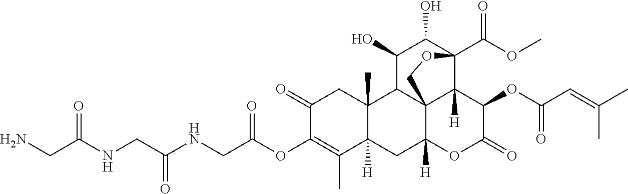 |
| | methyl (1R,2S,3S,3aS,3a1R,4R,6aR,7aR,11aS)-9-((glycylglycylglycyl)oxy)-1,2- |
| | dihydroxy-8,11a-dimethyl-4-((3-methylbut-2-enoyl)oxy)-5,10-dioxo- |
| | 1,4,5,6a,7,7a,10,11,11a,11b-decahydro-2H-3,3a1- |
| | (epoxymethano)dibenzo[de,g]chromene-3(3aH)-carboxylate |
| 24 | 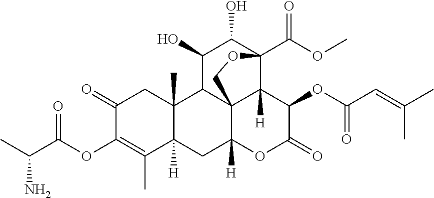 |
| | methyl (1R,2S,3S,3aS,3a1R,4R,6aR,7aR,11aS)-9-((D-alanyl)oxy)-1,2-dihydroxy- |
| | 8,11a-dimethyl-4-((3-methylbut-2-enoyl)oxy)-5,10-dioxo- |
| | 1,4,5,6a,7,7a,10,11,11a,11b-decahydro-2H-3,3a1- |
| | (epoxymethano)dibenzo[de,g]chromene-3(3aH)-carboxylate |
| 25 | 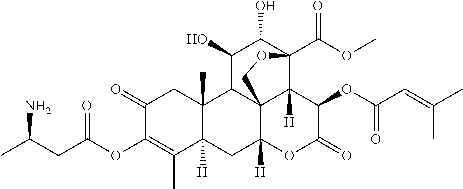 |
| | methyl (1R,2S,3S,3aS,3a1R,4R,6aR,7aR,11aS)-9-(((R)-3-aminobutanoyl)oxy)- |
| | 1,2-dihydroxy-8,11a-dimethyl-4-((3-methylbut-2-enoyl)oxy)-5,10-dioxo- |
| | 1,4,5,6a,7,7a,10,11,11a,11b-decahydro-2H-3,3a1- |
| | (epoxymethano)dibenzo[de,g]chromene-3(3aH)-carboxylate |
| 26 | 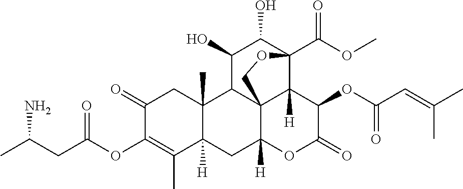 |
| | methyl (1R,2S,3S,3aS,3a1R,4R,6aR,7aR,11aS)-9-(((S)-3-aminobutanoyl)oxy)- |
| | 1,2-dihydroxy-8,11a-dimethyl-4-((3-methylbut-2-enoyl)oxy)-5,10-dioxo- |
| | 1,4,5,6a,7,7a,10,11,11a,11b-decahydro-2H-3,3a1- |
| | (epoxymethano)dibenzo[de,g]chromene-3(3aH)-carboxylate |
| 27 | 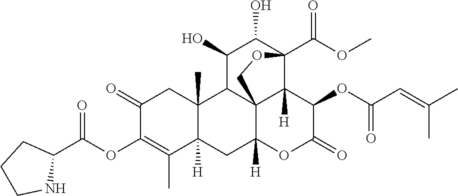 |
| | (1R,2S,3S,3aS,3a1R,4R,6aR,7aR,11aS)-1,2-dihydroxy-3-(methoxycarbonyl)- |
| | 8,11a-dimethyl-4-((3-methylbut-2-enoyl)oxy)-5,10-dioxo- |
| | 1,3,3a,4,5,6a,7,7a,10,11,11a,11b-dodecahydro-2H-3,3a1- |
| | (epoxymethano)dibenzo[de,g]chromen-9-yl D-prolinate |
| 28 | 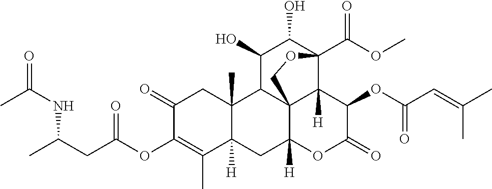 |
| | methyl (1R,2S,3S,3aS,3a1R,4R,6aR,7aR,11aS)-9-(((S)-3- |
| | acetamidobutanoyl)oxy)-1,2-dihydroxy-8,11a-dimethyl-4-((3-methylbut-2- |
| | enoyl)oxy)-5,10-dioxo-1,4,5,6a,7,7a,10,11,11a,11b-decahydro-2H-3,3a1- |
| | (epoxymethano)dibenzo[de,g]chromene-3(3aH)-carboxylate |
| 29 | 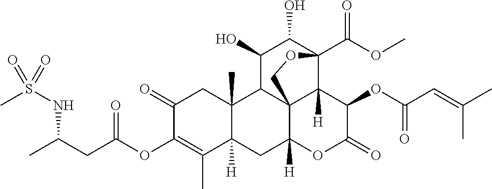 |
| | methyl (1R,2S,3S,3aS,3a1R,4R,6aR,7aR,11aS)-1,2-dihydroxy-8,11a-dimethyl-4- |
| | ((3-methylbut-2-enoyl)oxy)-9-(((S)-3-(methylsulfonamido)butanoyl)oxy)-5,10- |
| | dioxo-1,4,5,6a,7,7a,10,11,11a,11b-decahydro-2H-3,3a1- |
| | (epoxymethano)dibenzo[de,g]chromene-3(3aH)-carboxylate |
| 30 | 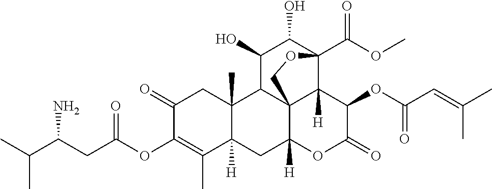 |
| | methyl (1R,2S,3S,3aS,3a1R,4R,6aR,7aR,11aS)-9-(((R)-3-amino-4- |
| | methylpentanoyl)oxy)-1,2-dihydroxy-8,11a-dimethyl-4-((3-methylbut-2- |
| | enoyl)oxy)-5,10-dioxo-1,4,5,6a,7,7a,10,11,11a,11b-decahydro-2H-3,3a1- |
| | (epoxymethano)dibenzo[de,g]chromene-3(3aH)-carboxylate |
| 31 | 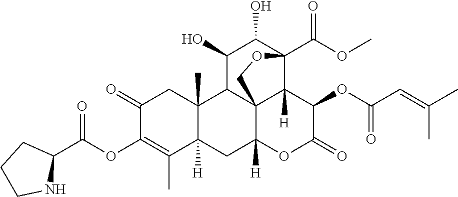 |
| | (1R,2S,3S,3aS,3a1R,4R,6aR,7aR,11aS)-1,2-dihydroxy-3-(methoxycarbonyl)- |
| | 8,11a-dimethyl-4-((3-methylbut-2-enoyl)oxy)-5,10-dioxo- |
| | 1,3,3a,4,5,6a,7,7a,10,11,11a,11b-dodecahydro-2H-3,3a1- |
| | (epoxymethano)dibenzo[de,g]chromen-9-yl L-prolinate |
| 32 | 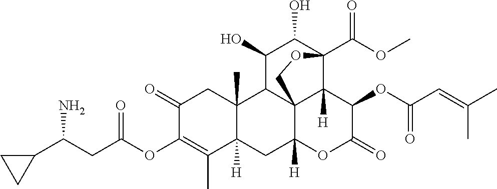 |
| | methyl (1R,2S,3S,3aS,3a1R,4R,6aR,7aR,11aS)-9-(((R)-3-amino-3- |
| | cyclopropylpropanoyl)oxy)-1,2-dihydroxy-8,11a-dimethyl-4-((3-methylbut-2- |
| | enoyl)oxy)-5,10-dioxo-1,4,5,6a,7,7a,10,11,11a,11b-decahydro-2H-3,3a1- |
| | (epoxymethano)dibenzo[de,g]chromene-3(3aH)-carboxylate |
| 33 | 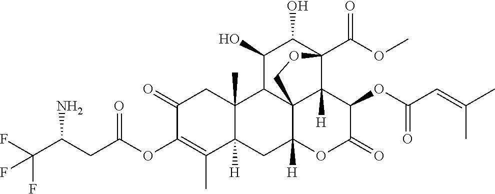 |
| | methyl (1R,2S,3S,3aS,3a1R,4R,6aR,7aR,11aS)-9-(((R)-3-amino-4,4,4- |
| | trifluorobutanoyl)oxy)-1,2-dihydroxy-8,11a-dimethyl-4-((3-methylbut-2- |
| | enoyl)oxy)-5,10-dioxo-1,4,5,6a,7,7a,10,11,11a,11b-decahydro-2H-3,3a1- |
| | (epoxymethano)dibenzo[de,g]chromene-3(3aH)-carboxylate |
| 34 | 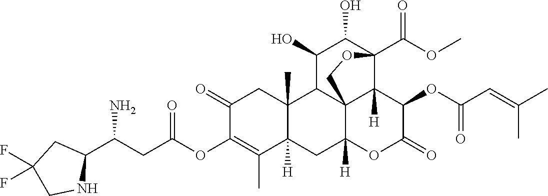 |
| | methyl (1R,2S,3S,3aS,3a1R,4R,6aR,7aR,11aS)-9-(((R)-3-amino-3-((S)-4,4- |
| | difluoropyrrolidin-2-yl)propanoyl)oxy)-1,2-dihydroxy-8,11a-dimethyl-4-((3- |
| | methylbut-2-enoyl)oxy)-5,10-dioxo-1,4,5,6a,7,7a,10,11,11a,11b-decahydro-2H- |
| | 3,3a1-(epoxymethano)dibenzo[de,g]chromene-3(3aH)-carboxylate |
| 39 | 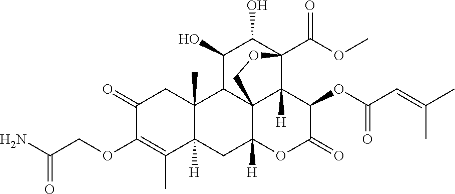 |
| | methyl (1R,2S,3S,3aS,3a1R,4R,6aR,7aR,11aS)-9-(2-amino-2-oxoethoxy)-1,2- |
| | dihydroxy-8,11a-dimethyl-4-((3-methylbut-2-enoyl)oxy)-5,10-dioxo- |
| | 1,4,5,6a,7,7a,10,11,11a,11b-decahydro-2H-3,3a1- |
| | (epoxymethano)dibenzo[de,g]chromene-3(3aH)-carboxylate |
| 40 | 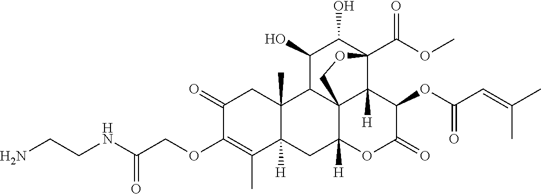 |
| | methyl (1R,2S,3S,3aS,3a1R,4R,6aR,7aR,11aS)-9-(2-((2-aminoethyl)amino)-2- |
| | oxoethoxy)-1,2-dihydroxy-8,11a-dimethyl-4-((3-methylbut-2-enoyl)oxy)-5,10- |
| | dioxo-1,4,5,6a,7,7a,10,11,11a,11b-decahydro-2H-3,3a1- |
| | (epoxymethano)dibenzo[de,g]chromene-3(3aH)-carboxylate |
| 41 | 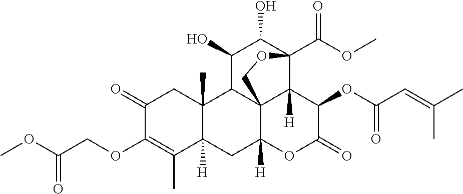 |
| | methyl (1R,2S,3S,3aS,3a1R,4R,6aR,7aR,11aS)-1,2-dihydroxy-9-(2-methoxy-2- |
| | oxoethoxy)-8,11a-dimethyl-4-((3-methylbut-2-enoyl)oxy)-5,10-dioxo- |
| | 1,4,5,6a,7,7a,10,11,11a,11b-decahydro-2H-3,3a1- |
| | (epoxymethano)dibenzo[de,g]chromene-3(3aH)-carboxylate |
| 42 | 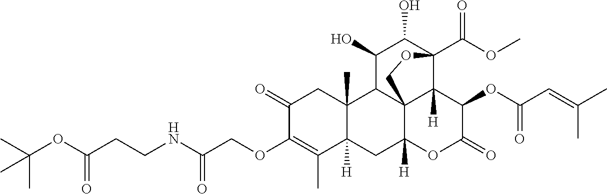 |
| | methyl (1R,2S,3S,3aS,3a1R,4R,6aR,7aR,11aS)-9-(2-((3-(tert-butoxy)-3- |
| | oxopropyl)amino)-2-oxoethoxy)-1,2-dihydroxy-8,11a-dimethyl-4-((3-methylbut- |
| | 2-enoyl)oxy)-5,10-dioxo-1,4,5,6a,7,7a,10,11,11a,11b-decahydro-2H-3,3a1- |
| | (epoxymethano)dibenzo[de,g]chromene-3(3aH)-carboxylate |
| 43 | 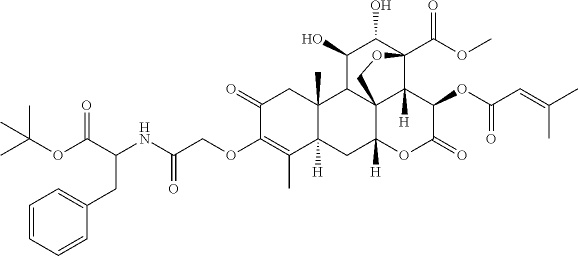 |
| | methyl (1R,2S,3S,3aS,3a1R,4R,6aR,7aR,11aS)-9-(2-((1-(tert-butoxy)-1-oxo-3- |
| | phenylpropan-2-yl)amino)-2-oxoethoxy)-1,2-dihydroxy-8,11a-dimethyl-4-((3- |
| | methylbut-2-enoyl)oxy)-5,10-dioxo-1,4,5,6a,7,7a,10,11,11a,11b-decahydro-2H- |
| | 3,3a1-(epoxymethano)dibenzo[de,g]chromene-3(3aH)-carboxylate |
| 44 | 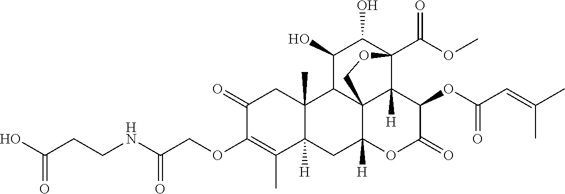 |
| | 3-(2-(((1R,2S,3S,3aS,3a1R,4R,6aR,7aR,11aS)-1,2-dihydroxy-3- |
| | (methoxycarbonyl)-8,11a-dimethyl-4-((3-methylbut-2-enoyl)oxy)-5,10-dioxo- |
| | 1,3,3a,4,5,6a,7,7a,10,11,11a,11b-dodecahydro-2H-3,3a1- |
| | (epoxymethano)dibenzo[de,g]chromen-9-yl)oxy)acetamido)propanoic acid |
| 45 | 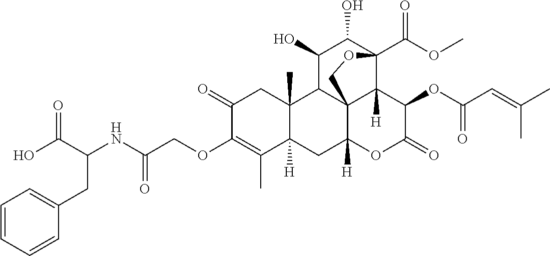 |
| | (2-(((1R,2S,3S,3aS,3a1R,4R,6aR,7aR,11aS)-1,2-dihydroxy-3- |
| | (methoxycarbonyl)-8,11a-dimethyl-4-((3-methylbut-2-enoyl)oxy)-5,10-dioxo- |
| | 1,3,3a,4,5,6a,7,7a,10,11,11a,11b-dodecahydro-2H-3,3a1- |
| | (epoxymethano)dibenzo[de,g]chromen-9-yl)oxy)acetyl)phenylalanine |
| 46 | 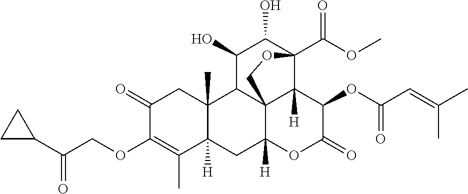 |
| | methyl (1R,2S,3S,3aS,3a1R,4R,6aR,7aR,11aS)-9-(2-cyclopropyl-2-oxoethoxy)- |
| | 1,2-dihydroxy-8,11a-dimethyl-4-((3-methylbut-2-enoyl)oxy)-5,10-dioxo- |
| | 1,4,5,6a,7,7a,10,11,11a,11b-decahydro-2H-3,3a1- |
| | (epoxymethano)dibenzo[de,g]chromene-3(3aH)-carboxylate |
| 47 | 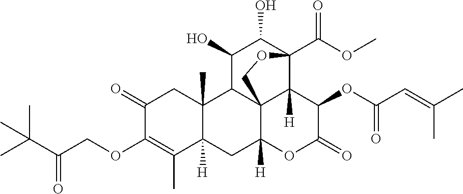 |
| | methyl (1R,2S,3S,3aS,3a1R,4R,6aR,7aR,11aS)-9-(3,3-dimethyl-2-oxobutoxy)- |
| | 1,2-dihydroxy-8,11a-dimethyl-4-((3-methylbut-2-enoyl)oxy)-5,10-dioxo- |
| | 1,4,5,6a,7,7a,10,11,11a,11b-decahydro-2H-3,3a1- |
| | (epoxymethano)dibenzo[de,g]chromene-3(3aH)-carboxylate |
| 48 | 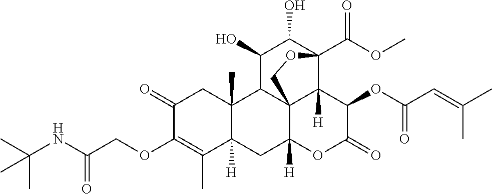 |
| | methyl (1R,2S,3S,3aS,3a1R,4R,6aR,7aR,11aS)-9-(2-(tert-butylamino)-2- |
| | oxoethoxy)-1,2-dihydroxy-8,11a-dimethyl-4-((3-methylbut-2-enoyl)oxy)-5,10- |
| | dioxo-1,4,5,6a,7,7a,10,11,11a,11b-decahydro-2H-3,3a1- |
| | (epoxymethano)dibenzo[de,g]chromene-3(3aH)-carboxylate |
| 49 | 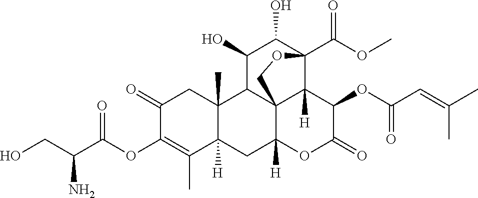 |
| | methyl (1R,2S,3S,3aS,3a1R,4R,6aR,7aR,11aS)-9-((L-seryl)oxy)-1,2-dihydroxy- |
| | 8,11a-dimethyl-4-((3-methylbut-2-enoyl)oxy)-5,10-dioxo- |
| | 1,4,5,6a,7,7a,10,11,11a,11b-decahydro-2H-3,3a1- |
| | (epoxymethano)dibenzo[de,g]chromene-3(3aH)-carboxylate |
| 50 | 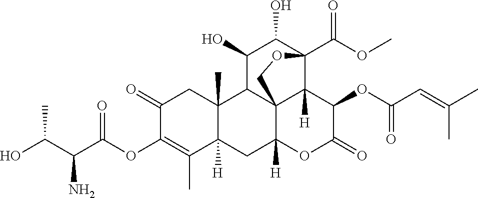 |
| | methyl (1R,2S,3S,3aS,3a1R,4R,6aR,7aR,11aS)-9-((L-threonyl)oxy)-1,2- |
| | dihydroxy-8,11a-dimethyl-4-((3-methylbut-2-enoyl)oxy)-5,10-dioxo- |
| | 1,4,5,6a,7,7a,10,11,11a,11b-decahydro-2H-3,3a1- |
| | (epoxymethano)dibenzo[de,g]chromene-3(3aH)-carboxylate |
| 51 | 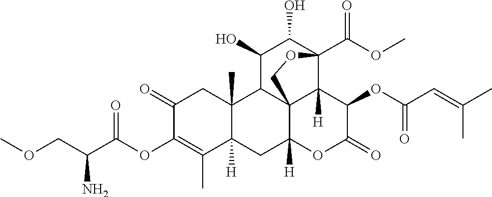 |
| | methyl (1R,2S,3S,3aS,3a1R,4R,6aR,7aR,11aS)-1,2-dihydroxy-8,11a-dimethyl-9- |
| | ((O-methyl-L-seryl)oxy)-4-((3-methylbut-2-enoyl)oxy)-5,10-dioxo- |
| | 1,4,5,6a,7,7a,10,11,11a,11b-decahydro-2H-3,3a1- |
| | (epoxymethano)dibenzo[de,g]chromene-3(3aH)-carboxylate |
| 52 | 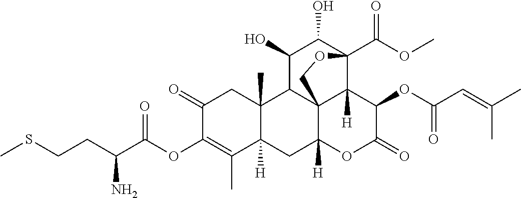 |
| | methyl (1R,2S,3S,3aS,3a1R,4R,6aR,7aR,11aS)-9-((L-methionyl)oxy)-1,2- |
| | dihydroxy-8,11a-dimethyl-4-((3-methylbut-2-enoyl)oxy)-5,10-dioxo- |
| | 1,4,5,6a,7,7a,10,11,11a,11b-decahydro-2H-3,3a1- |
| | (epoxymethano)dibenzo[de,g]chromene-3(3aH)-carboxylate |
| 53 | 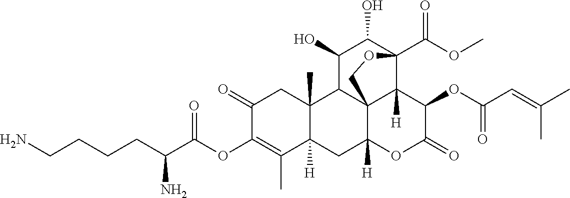 |
| | methyl (1R,2S,3S,3aS,3a1R,4R,6aR,7aR,11aS)-9-((L-methionyl)oxy)-1,2- |
| | dihydroxy-8,11a-dimethyl-4-((3-methylbut-2-enoyl)oxy)-5,10-dioxo- |
| | 1,4,5,6a,7,7a,10,11,11a,11b-decahydro-2H-3,3a1- |
| | (epoxymethano)dibenzo[de,g]chromene-3(3aH)-carboxylate |
| 54 | 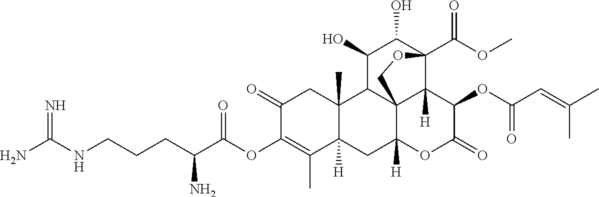 |
| | methyl (1R,2S,3S,3aS,3a1R,4R,6aR,7aR,11aS)-9-((L-arginyl)oxy)-1,2- |
| | dihydroxy-8,11a-dimethyl-4-((3-methylbut-2-enoyl)oxy)-5,10-dioxo- |
| | 1,4,5,6a,7,7a,10,11,11a,11b-decahydro-2H-3,3a1- |
| | (epoxymethano)dibenzo[de,g]chromene-3(3aH)-carboxylate |
| 55 | 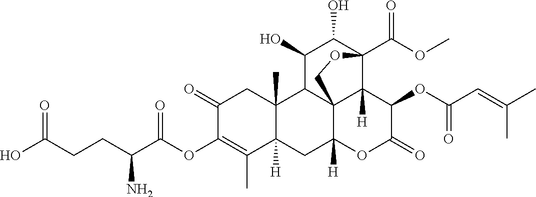 |
| | (4S)-4-amino-5-(((1R,2S,3S,3aS,3a1R,4R,6aR,7aR,11aS)-1,2-dihydroxy-3- |
| | (methoxycarbonyl)-8,11a-dimethyl-4-((3-methylbut-2-enoyl)oxy)-5,10-dioxo- |
| | 1,3,3a,4,5,6a,7,7a,10,11,11a,11b-dodecahydro-2H-3,3a1- |
| | (epoxymethano)dibenzo[de,g]chromen-9-yl)oxy)-5-oxopentanoic acid |
| 56 | 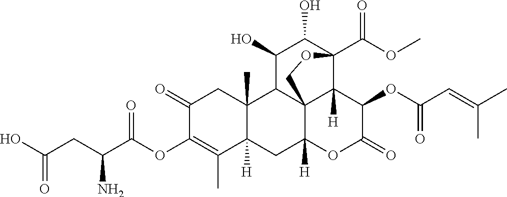 |
| | (3S)-3-amino-4-(((1R,2S,3S,3aS,3a1R,4R,6aR,7aR,11aS)-1,2-dihydroxy-3- |
| | (methoxycarbonyl)-8,11a-dimethyl-4-((3-methylbut-2-enoyl)oxy)-5,10-dioxo- |
| | 1,3,3a,4,5,6a,7,7a,10,11,11a,11b-dodecahydro-2H-3,3a1- |
| | (epoxymethano)dibenzo[de,g]chromen-9-yl)oxy)-4-oxobutanoic acid |
| 57 | 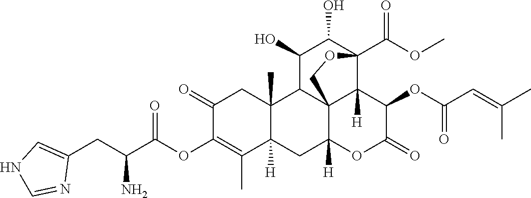 |
| | methyl (1R,2S,3S,3aS,3a1R,4R,6aR,7aR,11aS)-9-((L-histidyl)oxy)-1,2- |
| | dihydroxy-8,11a-dimethyl-4-((3-methylbut-2-enoyl)oxy)-5,10-dioxo- |
| | 1,4,5,6a,7,7a,10,11,11a,11b-decahydro-2H-3,3a1- |
| | (epoxymethano)dibenzo[de,g]chromene-3(3aH)-carboxylate |
| 58 | 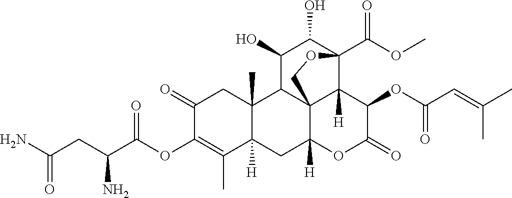 |
| | methyl (1R,2S,3S,3aS,3a1R,4R,6aR,7aR,11aS)-9-((L-asparaginyl)oxy)-1,2- |
| | dihydroxy-8,11a-dimethyl-4-((3-methylbut-2-enoyl)oxy)-5,10-dioxo- |
| | 1,4,5,6a,7,7a,10,11,11a,11b-decahydro-2H-3,3a1- |
| | (epoxymethano)dibenzo[de,g]chromene-3(3aH)-carboxylate |
| 59 | 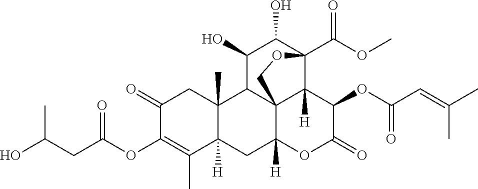 |
| | methyl (1R,2S,3S,3aS,3a1R,4R,6aR,7aR,11aS)-1,2-dihydroxy-9-((3- |
| | hydroxybutanoyl)oxy)-8,11a-dimethyl-4-((3-methylbut-2-enoyl)oxy)-5,10-dioxo- |
| | 1,4,5,6a,7,7a,10,11,11a,11b-decahydro-2H-3,3a1- |
| | (epoxymethano)dibenzo[de,g]chromene-3(3aH)-carboxylate |
| 51048 | 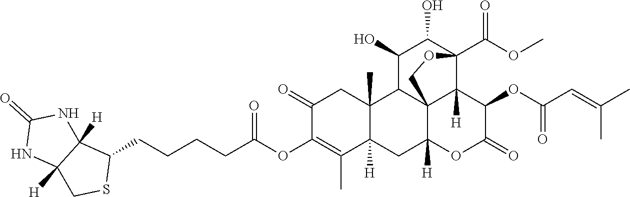 |
| | methyl (1R,2S,3S,3aS,3a1R,4R,6aR,7aR,11aS)-1,2-dihydroxy-8,11a-dimethyl-4- |
| | ((3-methylbut-2-enoyl)oxy)-5,10-dioxo-9-((5-((3aS,4S,6aR)-2-oxohexahydro- |
| | 1H-thieno[3,4-d]imidazol-4-yl)pentanoyl)oxy)-1,4,5,6a,7,7a,10,11,11a,11b- |
| | decahydro-2H-3,3a1-(epoxymethano)dibenzo[de,g]chromene-3(3aH)-carboxylate |
| 51052 | 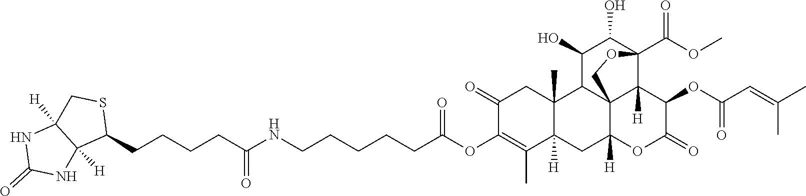 |
| | methyl (1R,2S,3S,3aS,3a1R,4R,6aR,7aR,11aS)-1,2-dihydroxy-8,11a-dimethyl-4- |
| | ((3-methylbut-2-enoyl)oxy)-5,10-dioxo-9-((6-(5-((3aS,4S,6aR)-2-oxohexahydro- |
| | 1H-thieno[3,4-d]imidazol-4-yl)pentanamido)hexanoyl)oxy)- |
| | 1,4,5,6a,7,7a,10,11,11a,11b-decahydro-2H-3,3a1- |
| | (epoxymethano)dibenzo[de,g]chromene-3(3aH)-carboxylate |
| 51046 | 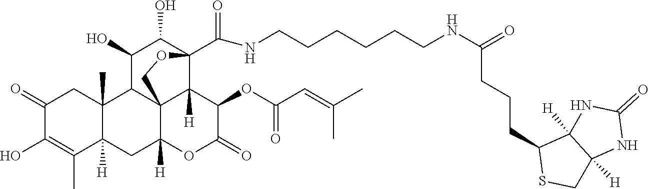 |
| | (1R,2S,3S,3aS,3a1R,4R,6aR,7aR,11aS)-1,2,9-trihydroxy-8,11a-dimethyl-5,10- |
| | dioxo-3-((6-(4-((3aS,4S,6aR)-2-oxohexahydro-1H-thieno[3,4-d]imidazol-4- |
| | yl)butanamido)hexyl)carbamoyl)-1,3,3a,4,5,6a,7,7a,10,11,11a,11b-dodecahydro- |
| | 2H-3,3a1-(epoxymethano)dibenzo[de,g]chromen-4-yl 3-methylbut-2-enoate |

### Enumerated Embodiments

The following enumerated embodiments are provided, the numbering of which is not to be construed as designating levels of importance.

Embodiment 1 provides a composition comprising a compound of formula (I):

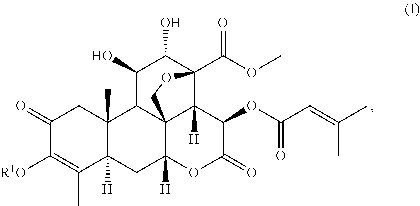

wherein:

R¹ is selected from the group consisting of

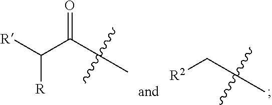

R and R′ are each independently selected from the group consisting of H, optionally substituted C₁-C₇ alkyl, optionally substituted C₁-C₆ aminoalkyl, optionally substituted C₃-C₇ cycloalkyl, optionally substituted C₆-C₁₀ aryl, optionally substituted benzyl, N(R^{a})(R^{b}), SR^{a}, OR^{a}, and C(═O)OR^{a},
or R and R′ combine with the atom to which they are bound to form

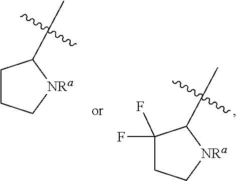

wherein each optional substituent in R and R′ is at least one selected from the group consisting of N(R^{a})(R^{b}), benzyl, C₁-C₆ alkyl, C₃-C₇ cycloalkyl, C₁-C₆ heterocycloalkyl, C(═O)C₁-C₃ alkyl, C(═O)C₃-C₇ cycloalkyl, C(═O)(C₁-C₃ alkyl)N(R^{a})(R^{b}), C₁-C₃ haloalkyl, halogen, C(═O)OR^{a}, OR^{a}, SR^{a}, imidazolyl, and N(R^{a})C(═NR^{e})N(R^{e})(R^{b});

R² is selected from the group consisting of C(═O)N(R^{e})(R^{c}), C(═O)OR^{a}, and C(═O)R^{a};

each occurrence of R^{a} and R^{b} is independently selected from the group consisting of H, C₁-C₆ alkyl, C₃-C₇ cycloalkyl, C(═O)C₁-C₆ alkyl, C(═O)O(C₁-C₆ alkyl), C(═O)(C₁-C₃ alkyl)NH₂, C(═O)(C₁-C₃ alkyl)NHC(═O)(C₁-C₃ alkyl)NH₂, and S(═O)₂(C₁-C₃ alkyl);

each occurrence of R^{c} is independently selected from the group consisting of H, C₁-C₃ alkyl-N(R^{a})(R^{b}), C₁-C₃ alkyl-C(═O)OR^{a}, and CH(CO₂R^{a})CH₂Ph;

each occurrence of R^{e} is independently selected from the group consisting of H, C₁-C₆ alkyl, and C₃-C₇ cycloalkyl;

or a pharmaceutically acceptable salt, solvate, or polymorph thereof.

Embodiment 2 provides the composition of Embodiment 1, wherein R is C₁-C₃ alkyl optionally substituted with NH₂ or NHBoc.

Embodiment 3 provides the composition of any of Embodiments 1-2, wherein the compound is selected from the group consisting of:

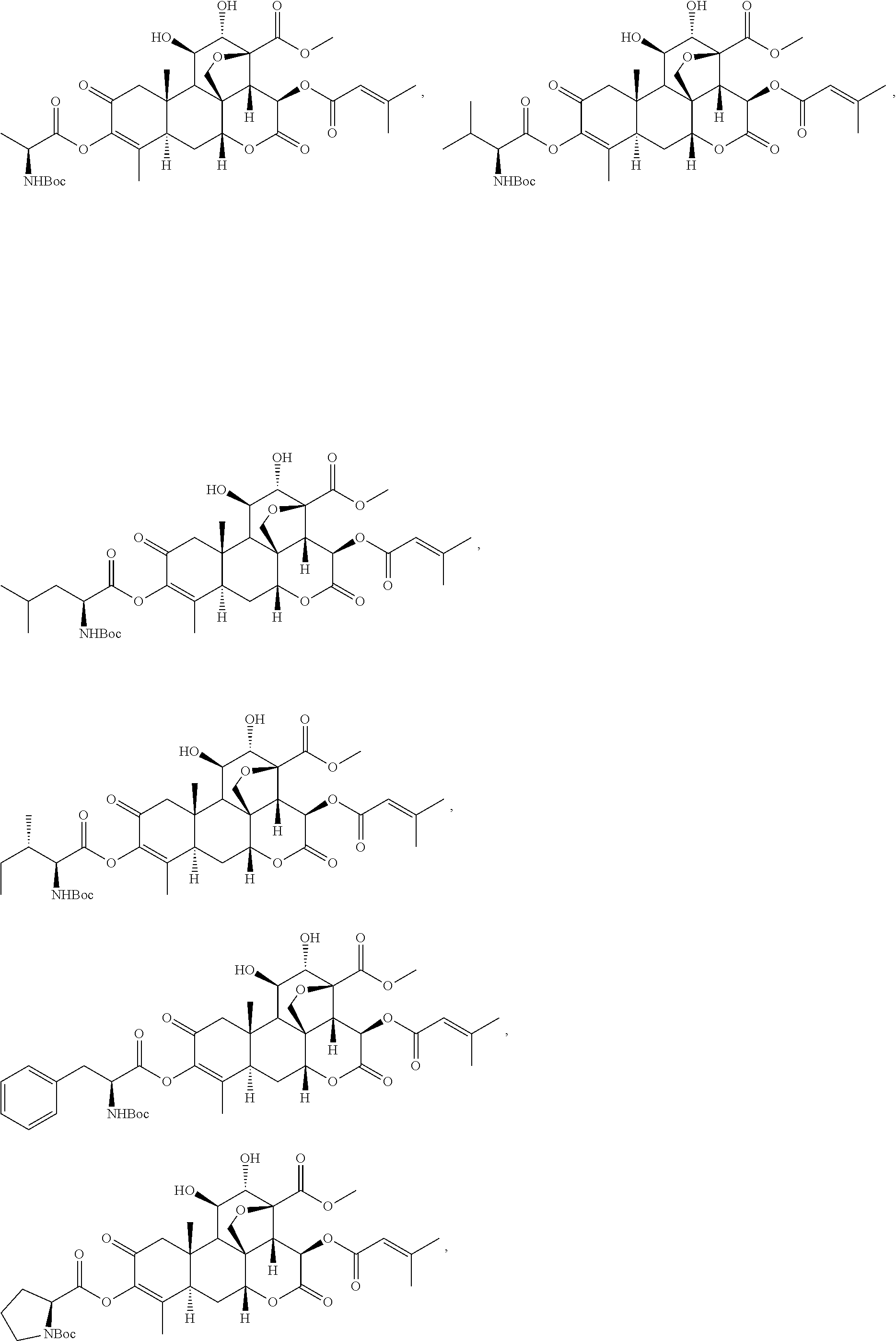

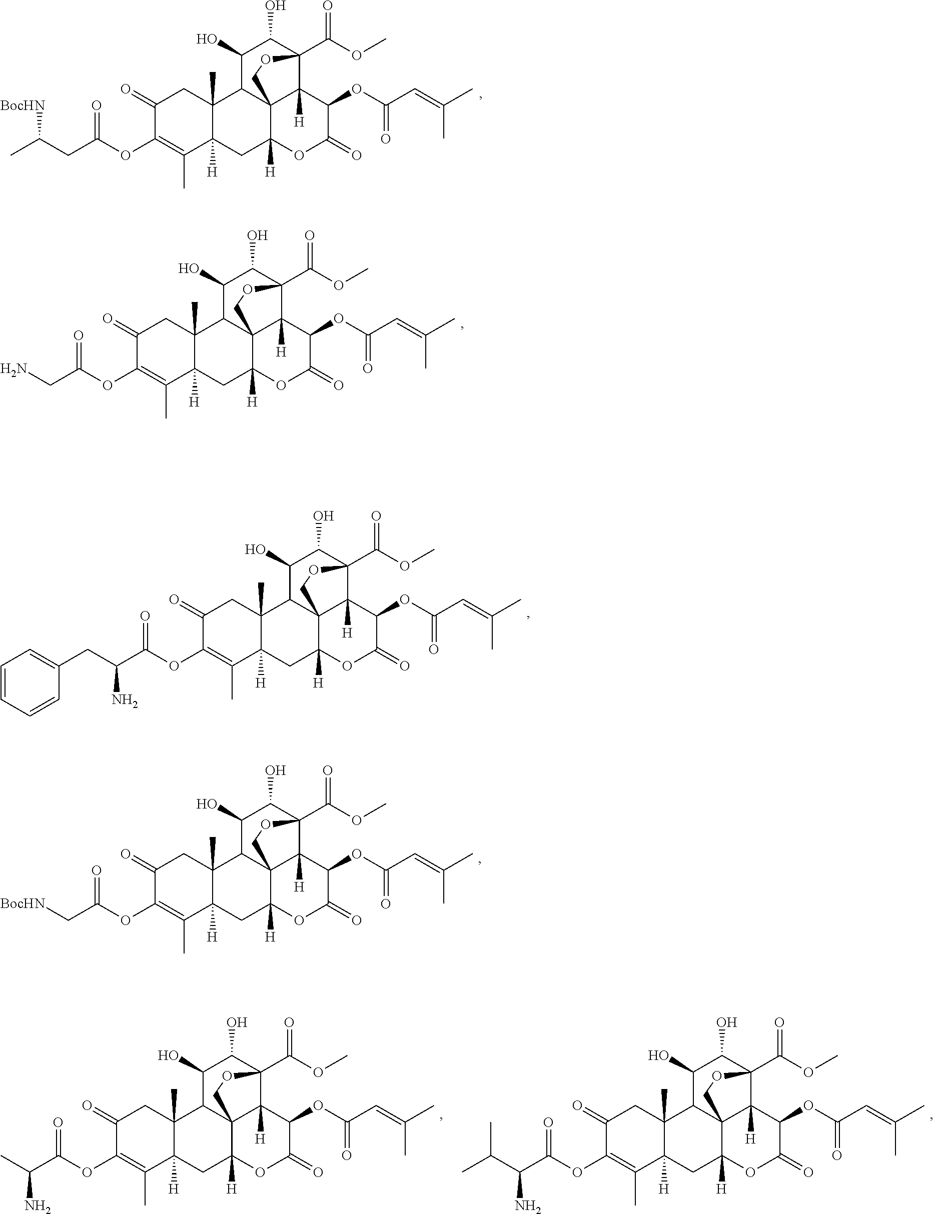

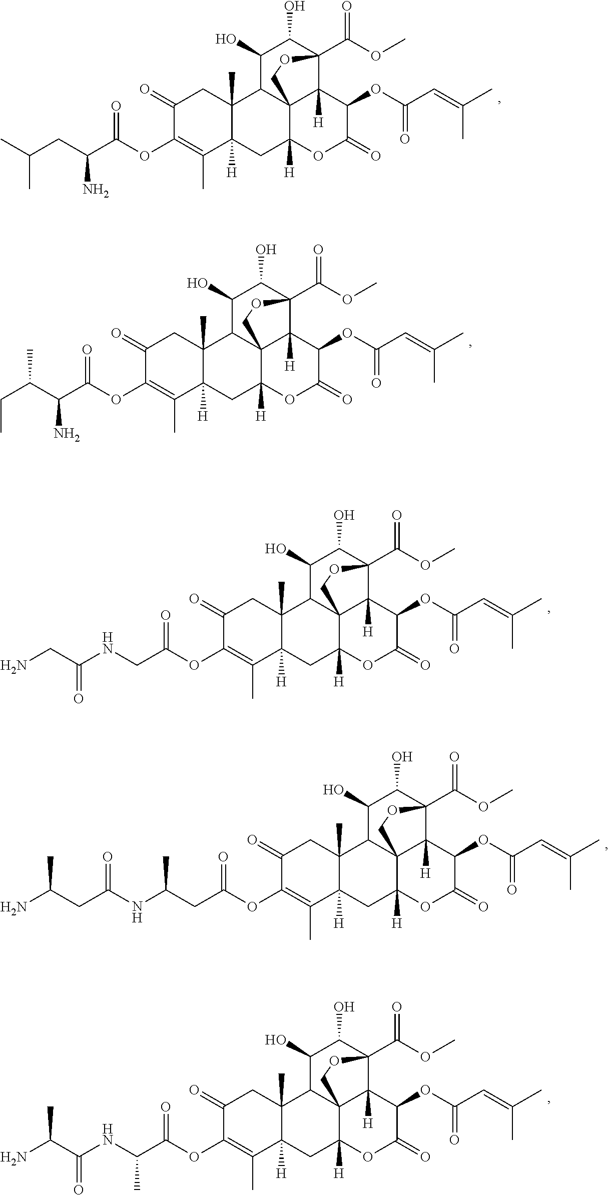

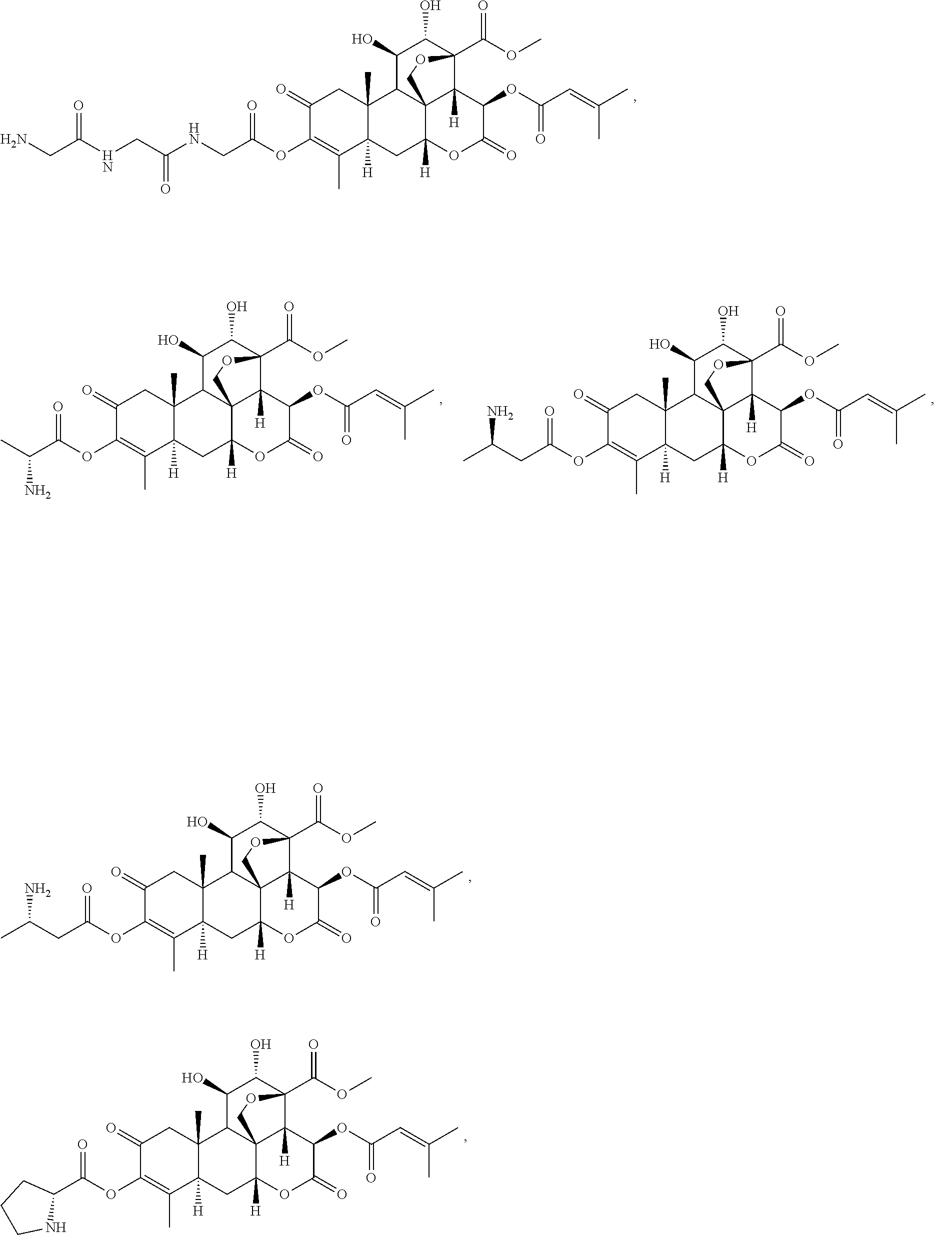

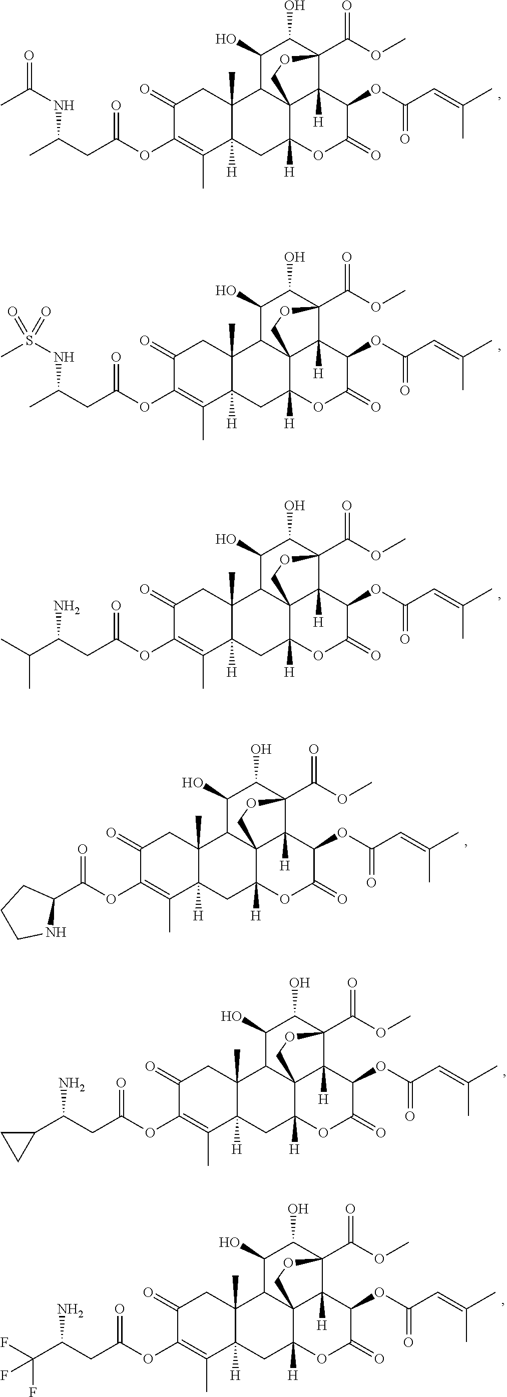

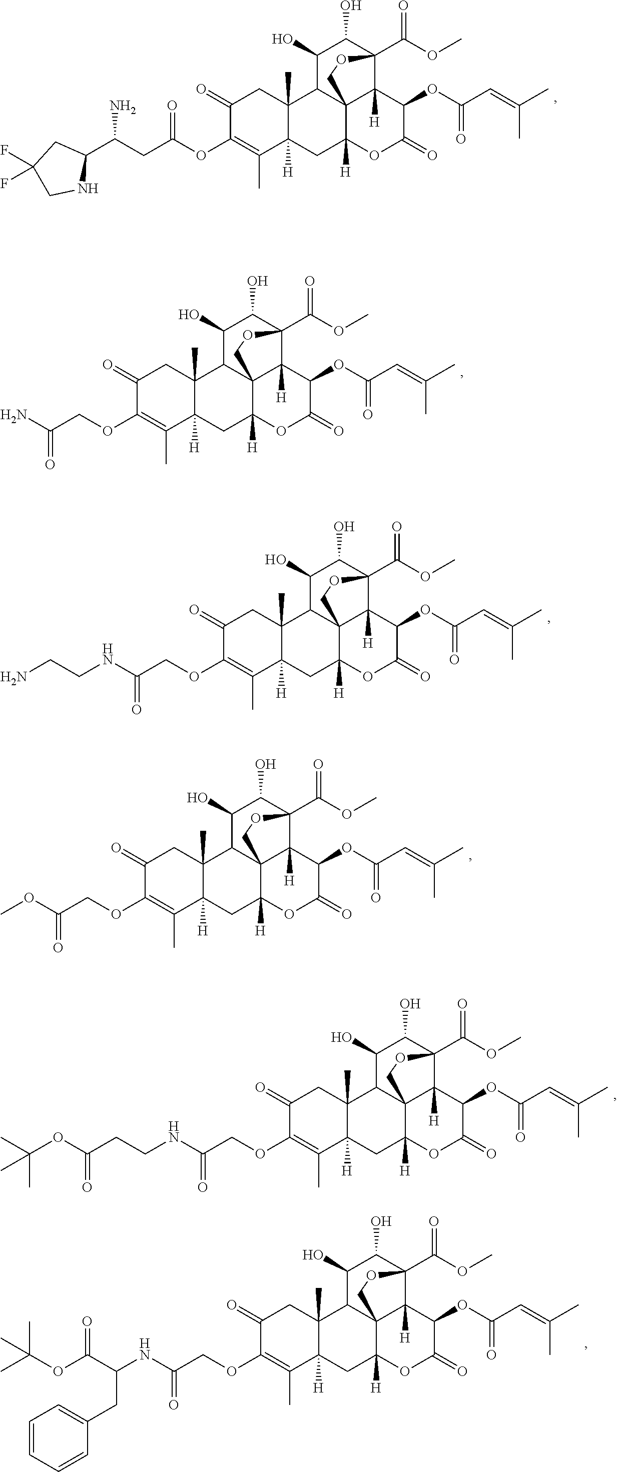

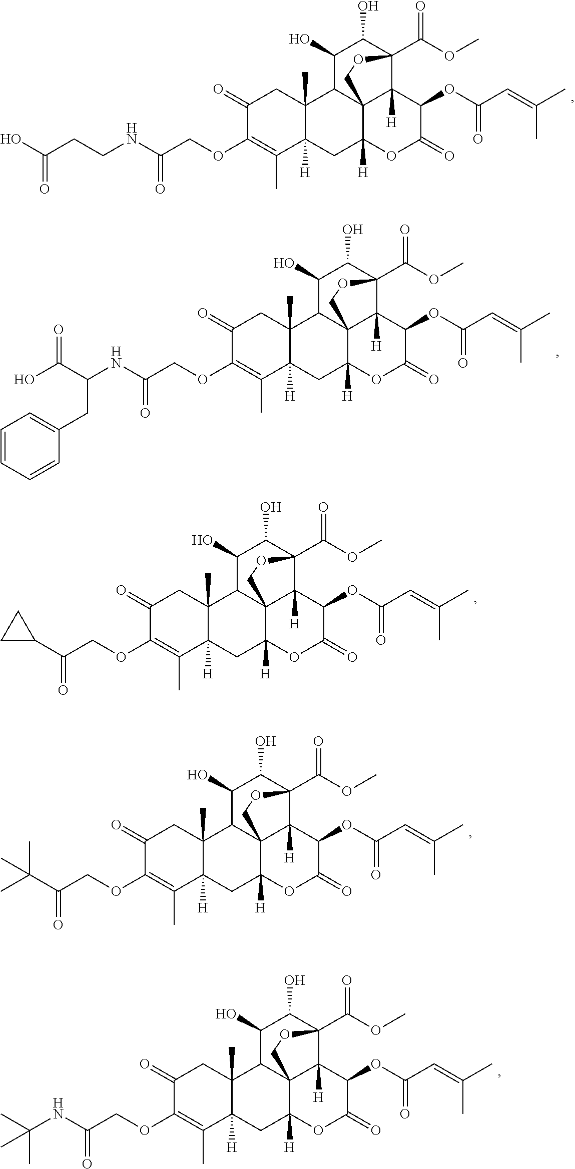

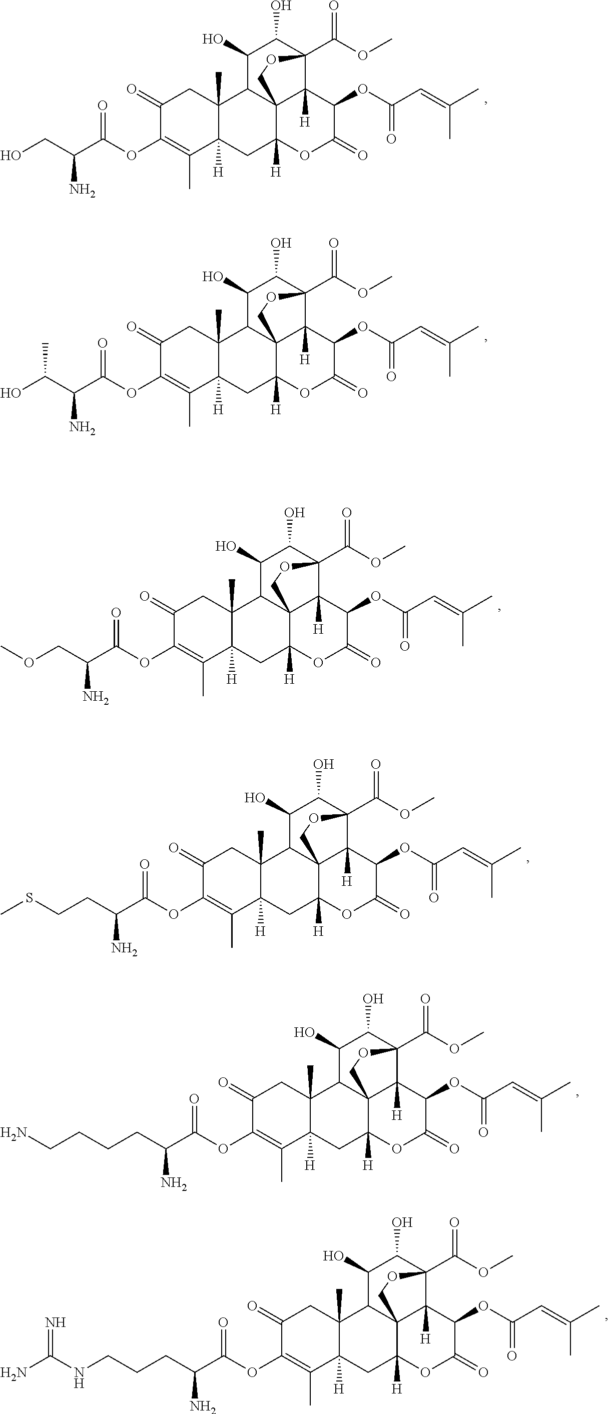

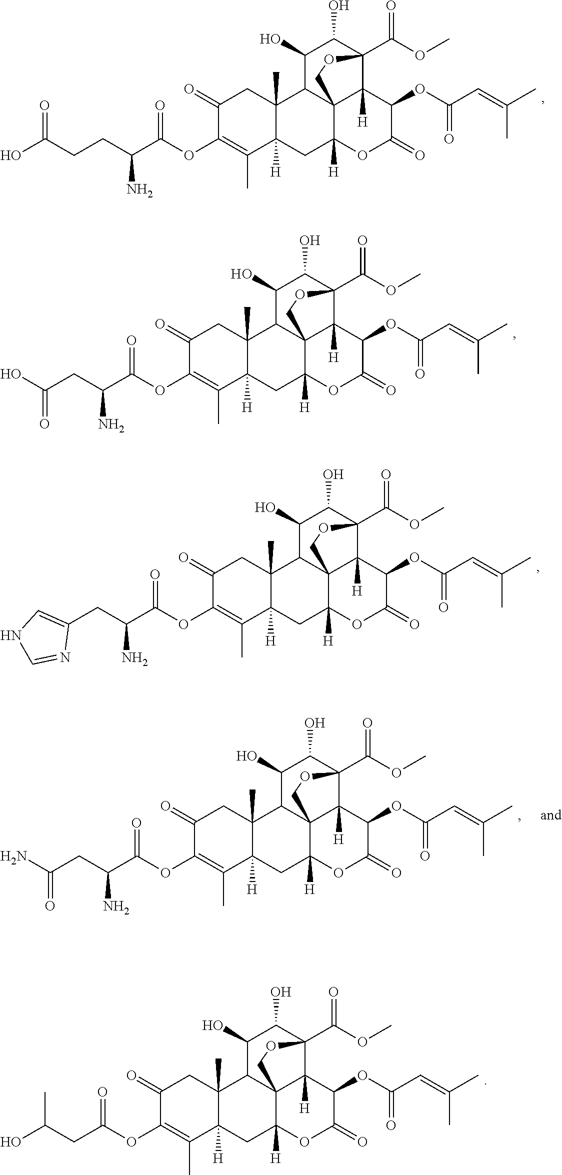

Embodiment 4 provides a composition comprising a compound of formula (Ib)

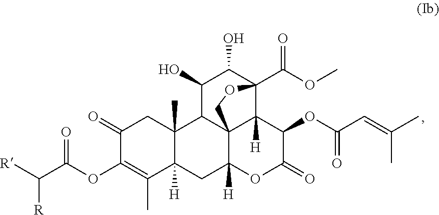

wherein R and R′ are each independently selected from the group consisting of optionally substituted C₁-C₆ alkyl, benzyl, NH₂, NHBoc and H,

or R and R′ combine with the atom to which they are bound to form

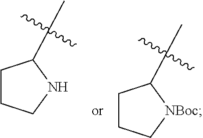 [SMILES: *C1CCCN1 ; *C1CCCN1C(=O)OC(C)(C)C]

or a pharmaceutically acceptable salt, solvate or polymorph thereof.

Embodiment 5 provides the composition of Embodiment 4, wherein R is C₁-C₃ alkyl optionally substituted with NH₂ or NHBoc.

Embodiment 6 provides the composition of any of Embodiments 4-5, wherein the compound is selected from the group consisting of:

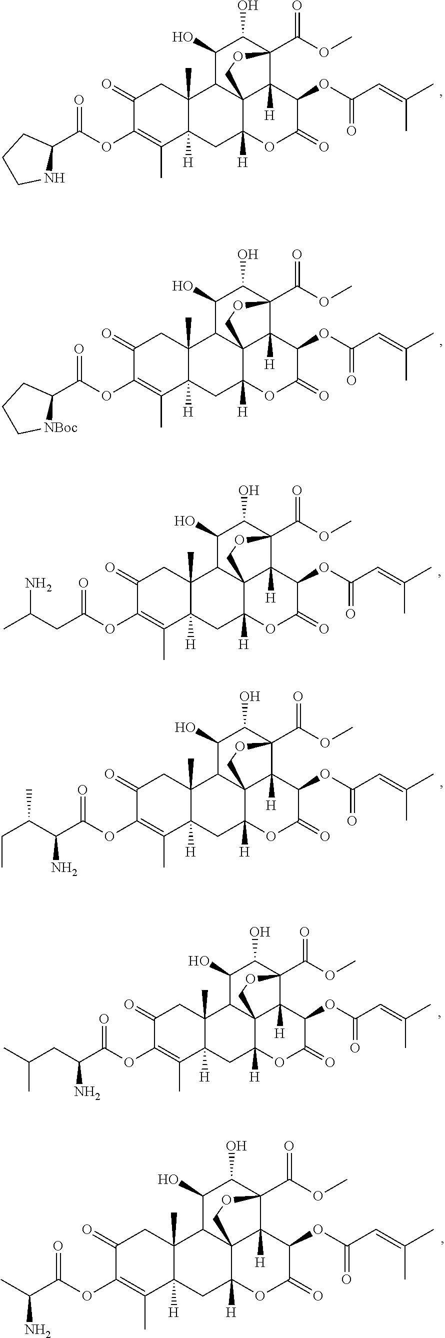

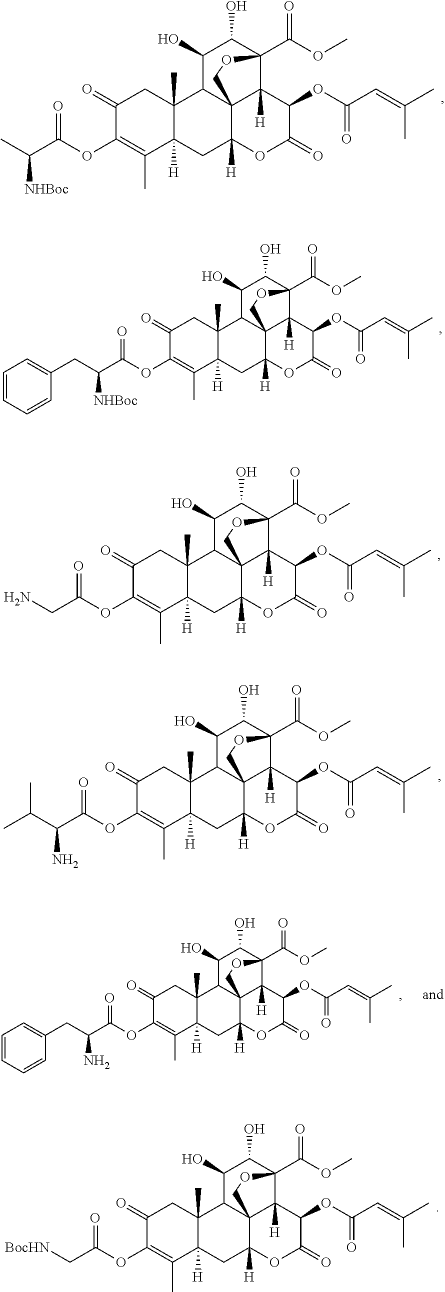

Embodiment 7 provides the composition of any of Embodiments 4-6, wherein the compound is selected from the group consisting of:

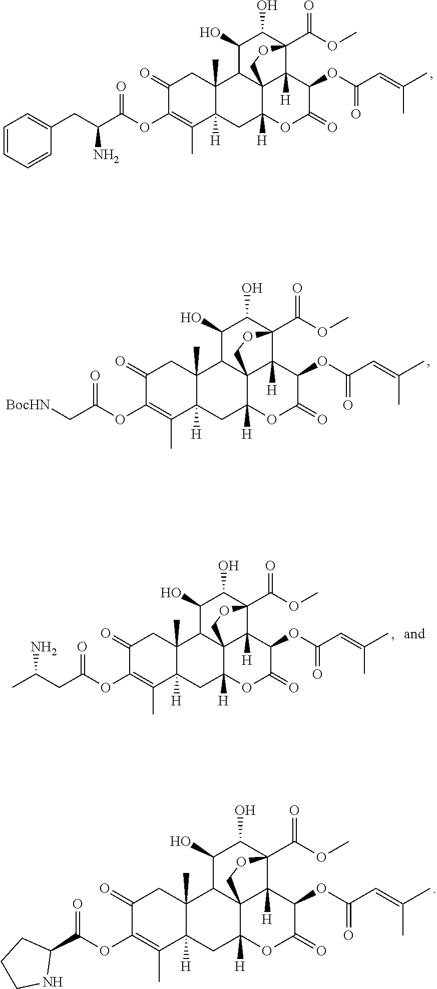

Embodiment 8 provides a composition comprising a compound of formula (IIa):

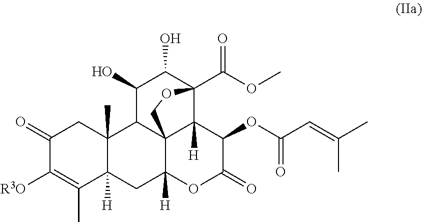

wherein:

R³ is selected from the group consisting of

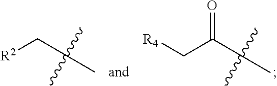

R² is selected from the group consisting of C(═O)N(R^{e})(R^{c}), C(═O)OR^{a}, and C(═O)R^{a};

R⁴ is selected from the group consisting of C₁-C₇ alkyl, C₁-C₇ cycloalkyl, C₁-C₃ aminoalkyl, C₆-C₁₀ aryl, benzyl, OR^{a}, N(R^{a})(R^{b}), SR^{a}, and C(═O)OR^{a},
wherein the C₁-C₇ alkyl, C₁-C₇ alkyl, C₁-C₃ aminoalkyl, C₆-C₁₀ aryl, or benzyl in R⁴ is substituted with at least one substituent selected from the group consisting of OR^{a}, SR^{a}, N(R^{a})(R^{d}), S(═O)₂(C₁-C₃ alkyl), C₃-C₇ cycloalkyl, C₁-C₆ heterocycloalkyl, and halogen;

each occurrence of R^{a} and R^{b} is independently selected from the group consisting of H, C₁-C₆ alkyl, C₃-C₇ cycloalkyl, C(═O)O(C₁-C₆ alkyl), C(═O)(C₁-C₃ alkyl)NH₂, and S(═O)₂(C₁-C₃ alkyl);

each occurrence of R^{c} is independently selected from the group consisting of H, C₁-C₃ alkyl-N(R^{a})(R^{b}), C₁-C₃ alkyl-C(═O)OR^{a}, and CH(CO₂R^{a})CH₂Ph;

each occurrence of R^{d} is independently selected from the group consisting of C₁-C₆ alkyl, C₃-C₇ cycloalkyl, C(═O)C₁-C₆ alkyl, C(═O)O(C₁-C₆ alkyl), C(═O)(C₁-C₃ alkyl)NH₂, and S(═O)₂(C₁-C₃); and

each occurrence of R^{c} is independently selected from the group consisting of H, C₁-C₆ alkyl, and C₃-C₇ cycloalkyl;

or a pharmaceutically acceptable salt, solvate, or polymorph thereof.

Embodiment 9 provides the composition of Embodiment 8, wherein the compound is selected from the group consisting of:

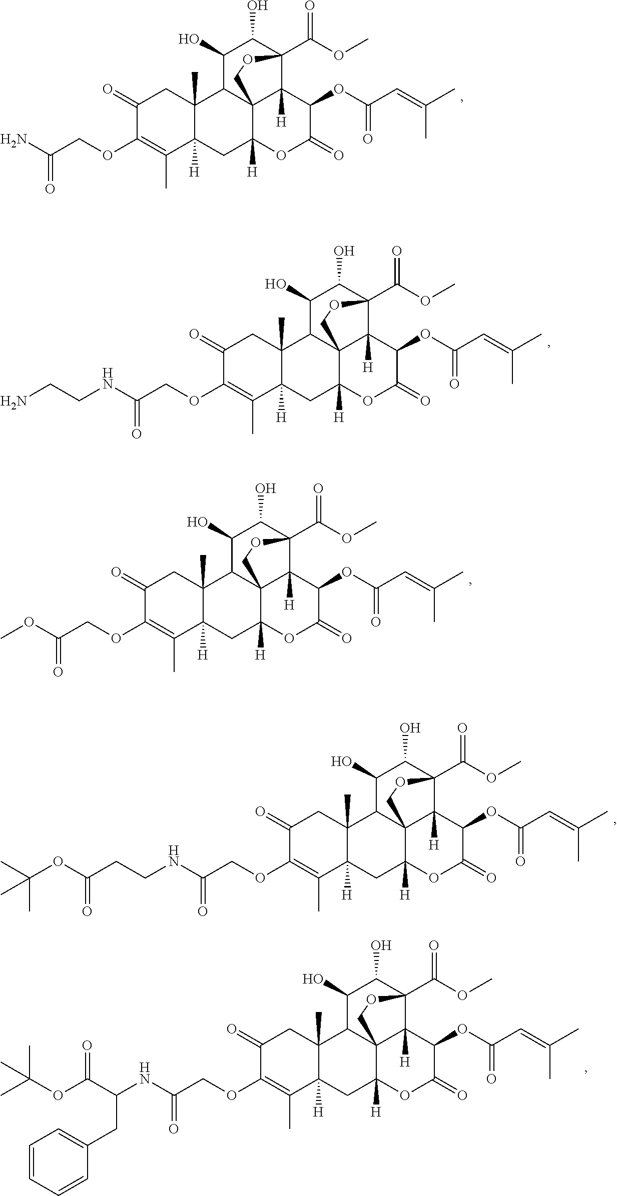

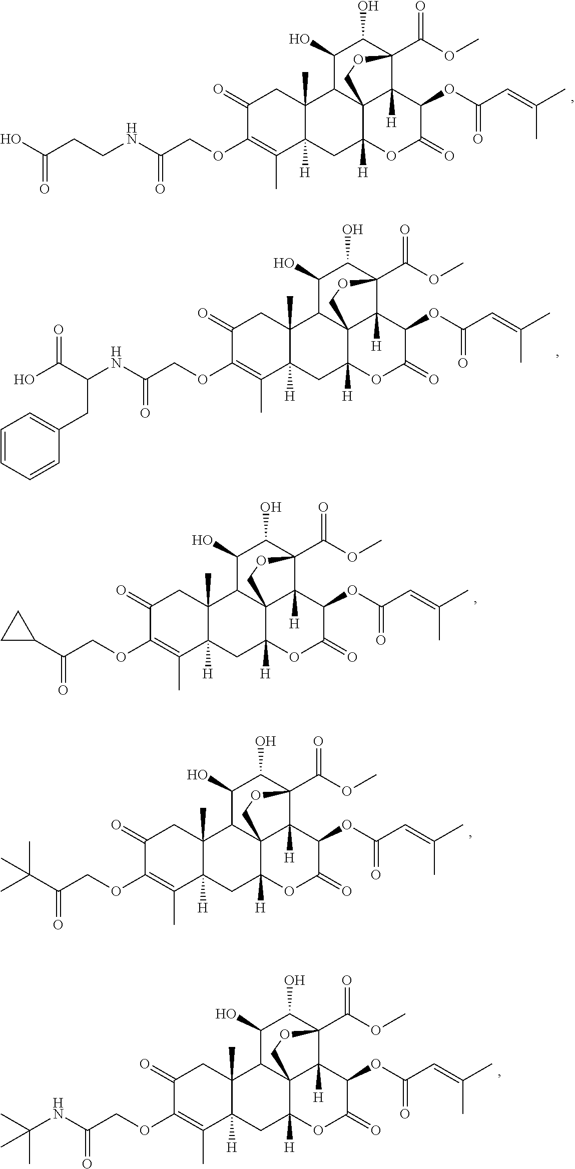

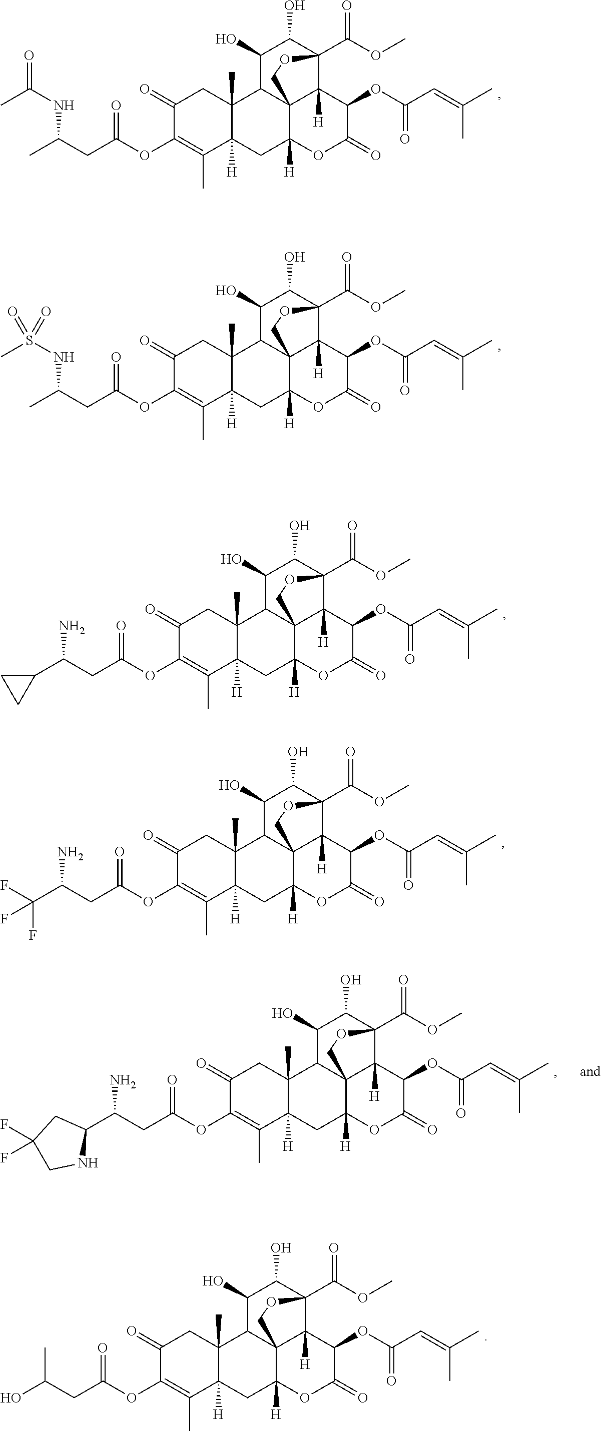

Embodiment 10 provides a composition comprising a compound of formula (IIb):

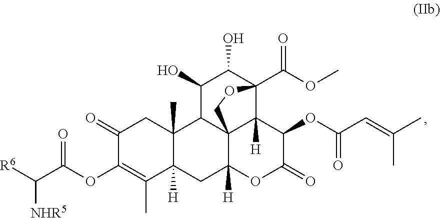

wherein:

R⁵ is selected from the group consisting of H, C₁-C₆ alkyl, and C(═O)O(C₁-C₆ alkyl);

R⁶ is selected from the group consisting of C₁-C₇ alkyl, C₁-C₇ cycloalkyl, C₆-C₁₀ aryl, benzyl, OR^{a}, N(R^{a})(R^{b}), SR^{a}, and C(═O)OR^{a},
wherein the C₁-C₇ alkyl, C₁-C₇ alkyl, C₆-C₁₀ aryl, or benzyl in R⁵ is substituted with at least one substituent selected from the group consisting of C₃-C₇ cycloalkyl, C₁-C₆ heterocycloalkyl, OR^{a}, SR^{a}, N(R^{a})(R^{b}), NR^{a}C(═NR^{a})N(R^{a})(R^{b}), C(═O)OR^{a}, C(═O)N(R^{a})(R^{b}), halogen, and imidazolyl; and

each occurrence of R^{a} and R^{b} is independently selected from the group consisting of H, C₁-C₆ alkyl, C₃-C₇ cycloalkyl, C(═O)C₁-C₆ alkyl, C(═O)O(C₁-C₆ alkyl), C(═O)(C₁-C₃ alkyl)NH₂, and S(═O)₂(C₁-C₃ alkyl);

or a pharmaceutically acceptable salt, solvate, or polymorph thereof.

Embodiment 11 provides the composition of Embodiment 10, wherein the compound is selected from the group consisting of:

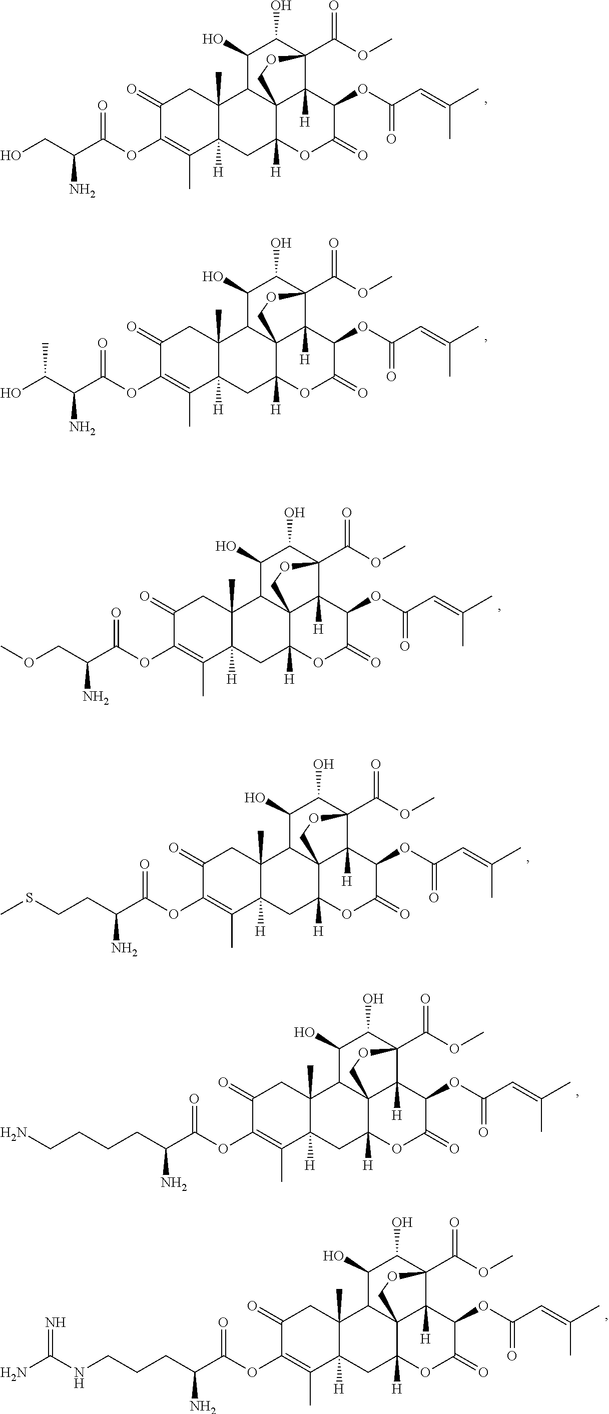

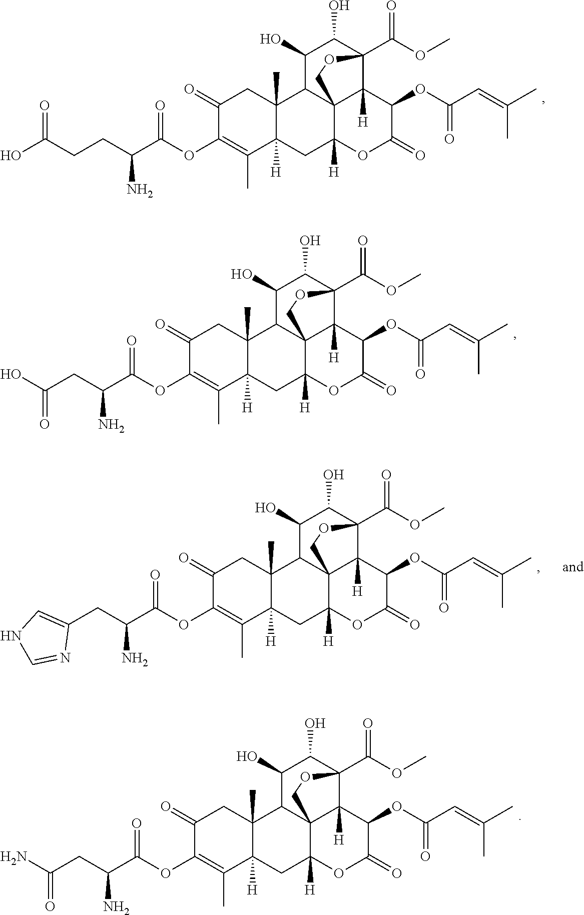

Embodiment 12 provides the composition of any of Embodiments 1-11, further comprising at least one pharmaceutically acceptable carrier.

Embodiment 13 provides a method of treating a hematologic malignancy in a subject in need thereof, the method comprising administering to the subject a therapeutically effective amount of the composition of Embodiment 12.

Embodiment 14 provides the method of Embodiment 13, wherein the hematologic malignancy is selected from the group consisting of leukemia and lymphoma.

Embodiment 15 provides the method of any of Embodiments 13-14, wherein the hematologic malignancy is selected from the group consisting of acute myeloid leukemia, Burkitt's lymphoma, B-cell lymphoma, hepatocellular carcinoma, acute promyelocytic leukemia, plasmacytoma, myeloma, chronic myelogenous leukemia, Epstein-Barr Virus (EBV) associated lymphoma, acute lymphoblastic leukemia, acute promyelocytic leukemia, large cell lymphoma, mantle cell lymphoma, and non-Hodgkin's B-cell lymphoma.

Embodiment 16 provides the method of any of Embodiments 13-15, wherein the hematologic malignancy is Epstein-Barr Virus (EBV) associated lymphoma.

Embodiment 17 provides the method of any of Embodiments 13-16, wherein the subject is a mammal.

Embodiment 18 provides the method of Embodiment 17, wherein the mammal is a human.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A composition comprising a compound of formula (I): 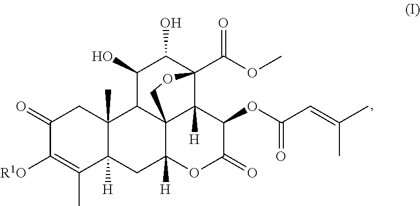
wherein:
R¹ is selected from the group consisting of 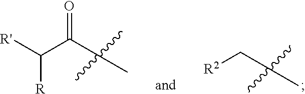
R and R′ are each independently selected from the group consisting of H, optionally substituted C₁-C₇ alkyl, optionally substituted C₁-C₆ aminoalkyl, optionally substituted C₃-C₇ cycloalkyl, optionally substituted C₆-C₁₀ aryl, optionally substituted benzyl, N(R^{a})(R^{b}), SR^{a}, OR^{a}, and C(═O)OR^{a},
or R and R′ combine with the atom to which they are bound to form 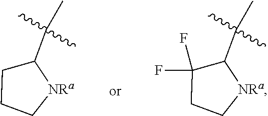
wherein each optional substituent in R and R′ is at least one selected from the group consisting of N(R^{a})(R^{b}), benzyl, C₁-C₆ alkyl, C₃-C₇ cycloalkyl, C₁-C₆ heterocycloalkyl, C(═O)C₁-C₃ alkyl, C(═O)C₃-C₇ cycloalkyl, C(═O)(C₁-C₃ alkyl)N(R^{a})(R^{b}), C₁-C₃ haloalkyl, halogen, C(═O)OR^{a}, OR^{a}, SR^{a}, imidazolyl, and N(R^{a})C(═NR^{e})N(R^{e})(R^{b});
R² is selected from the group consisting of C(═O)N(R^{e})(R^{c}), C(═O)OR^{a}, and C(═O)R^{a};
each occurrence of R^{a} and R^{b} is independently selected from the group consisting of H, C₁-C₆ alkyl, C₃-C₇ cycloalkyl, C(═O)C₁-C₆ alkyl, C(═O)O(C₁-C₆ alkyl), C(═O)(C₁-C₃ alkyl)NH₂, C(═O)(C₁-C₃ alkyl)NHC(═O)(C₁-C₃ alkyl)NH₂, and S(═O)₂(C₁-C₃ alkyl);
each occurrence of R^{c} is independently selected from the group consisting of H, C₁-C₃ alkyl-N(R^{a})(R^{b}), C₁-C₃ alkyl-C(═O)OR^{a}, and CH(CO₂R^{a})CH₂Ph;
each occurrence of R^{e} is independently selected from the group consisting of H, C₁-C₆ alkyl, and C₃-C₇ cycloalkyl;
or a pharmaceutically acceptable salt, solvate, or polymorph thereof.

2. The composition of claim 1, wherein R is C₁-C₃ alkyl optionally substituted with NH₂ or NHBoc.

3. The composition of claim 1, wherein the compound is selected from the group consisting of:

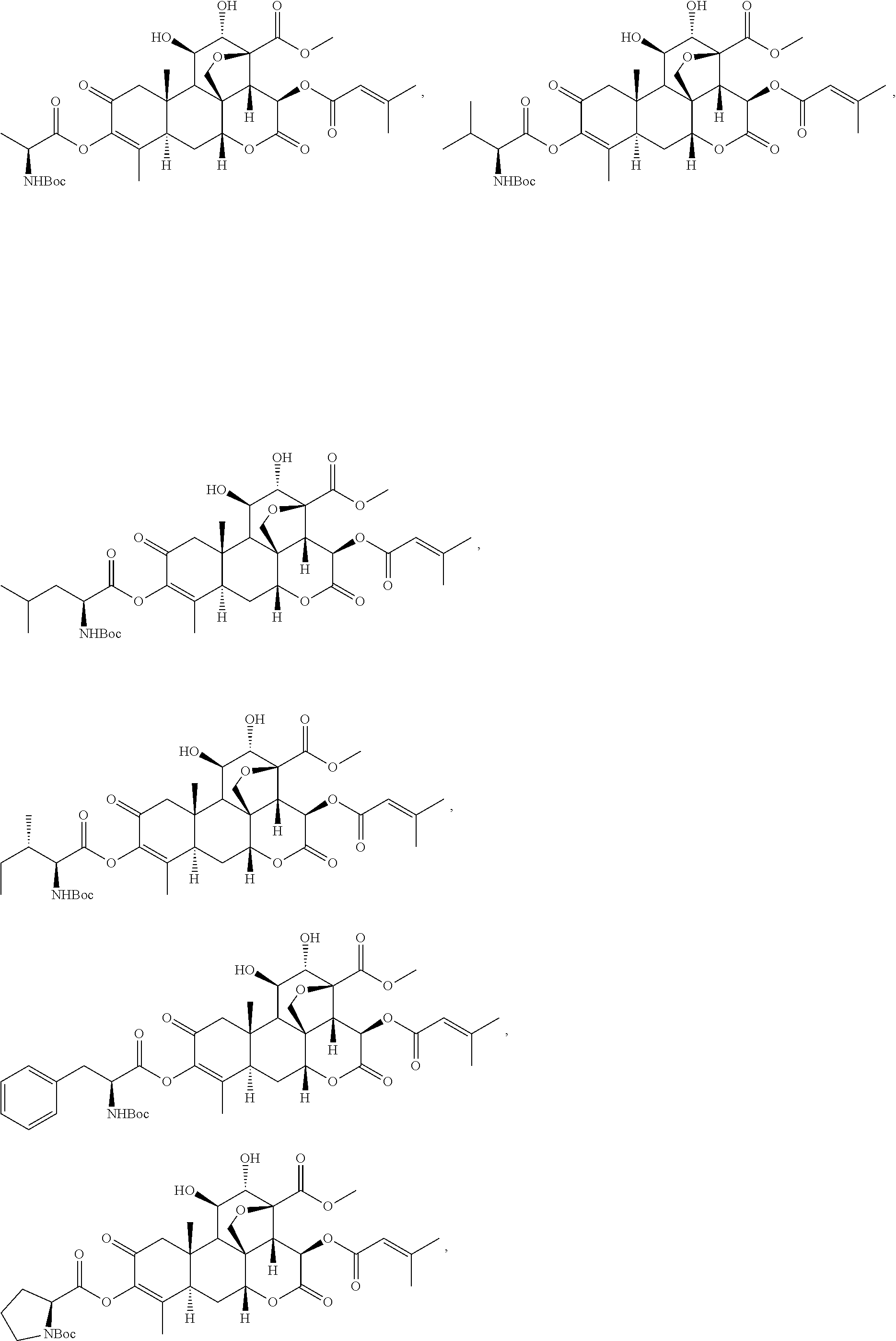

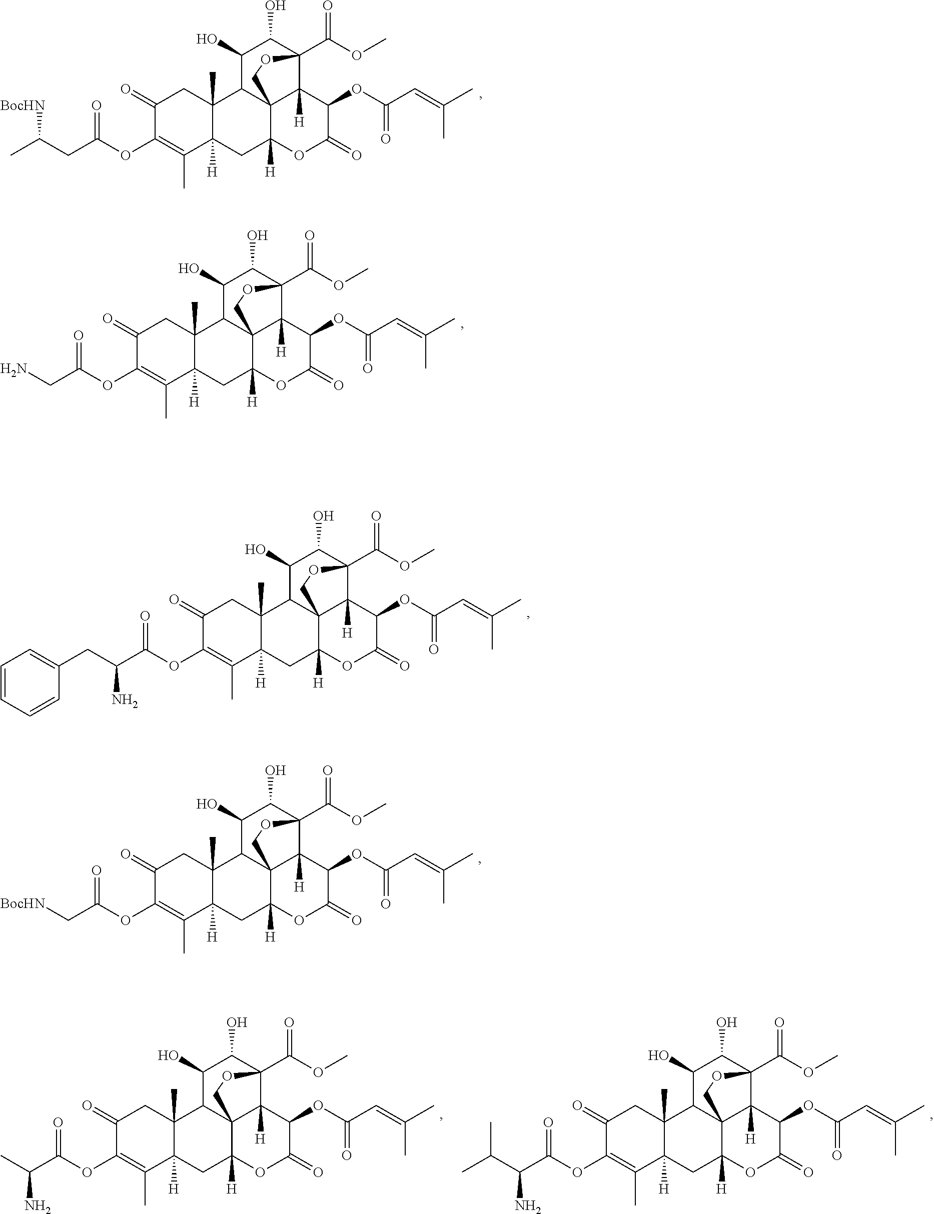

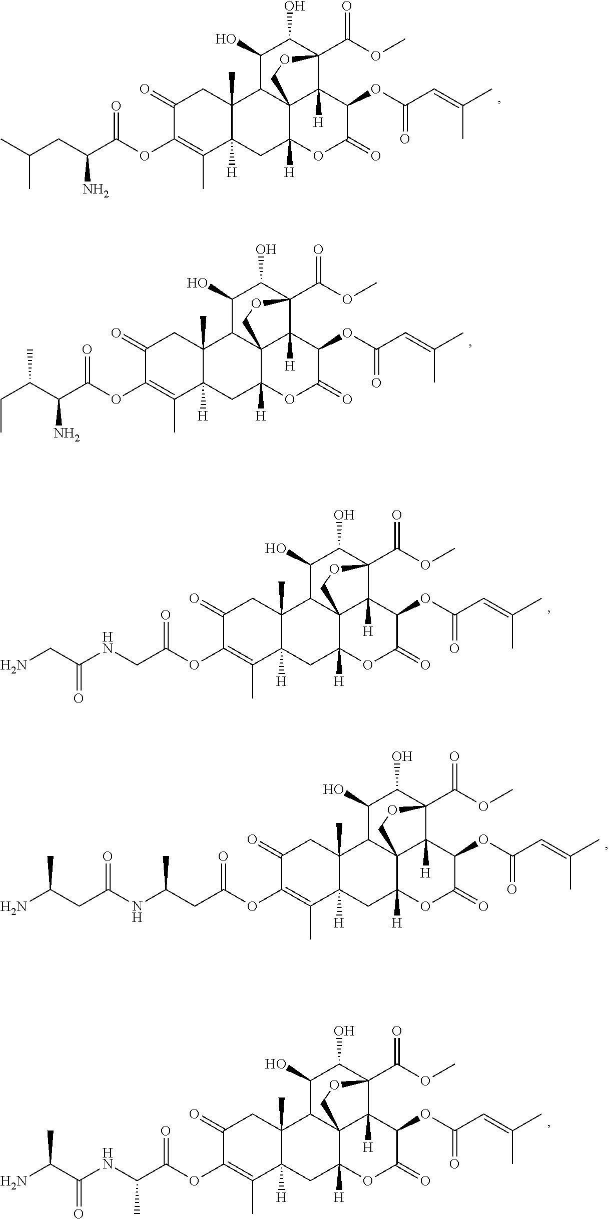

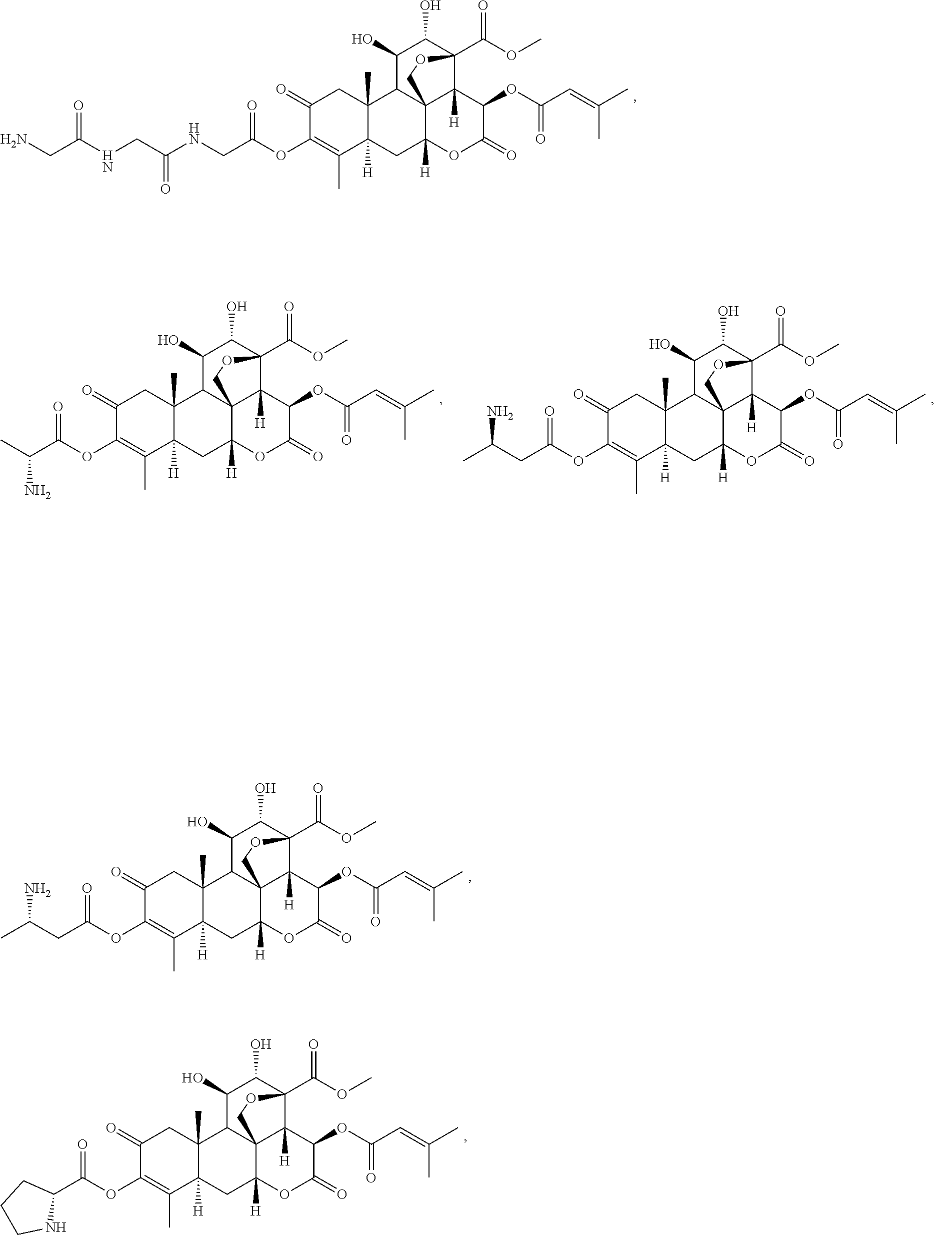

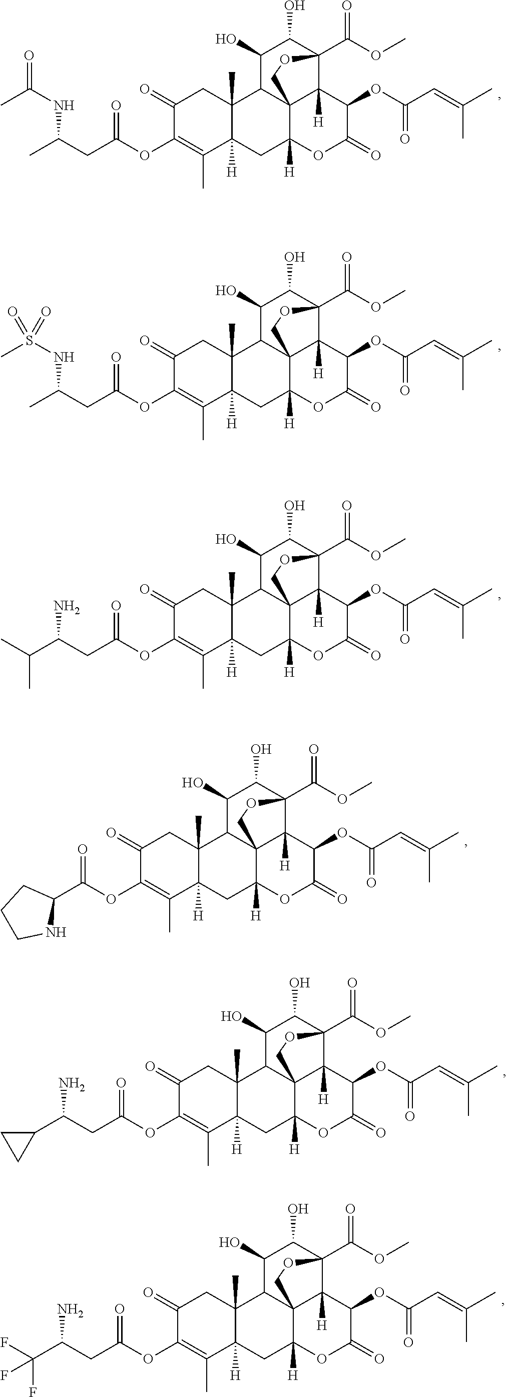

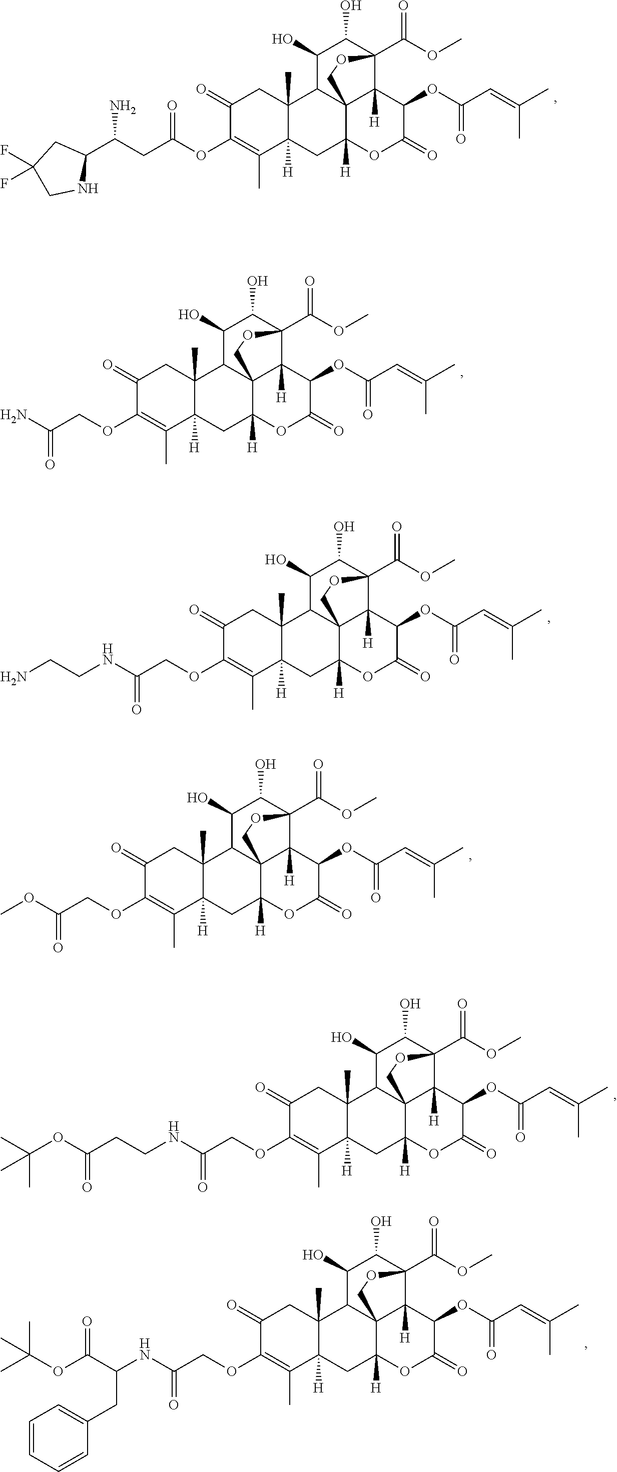

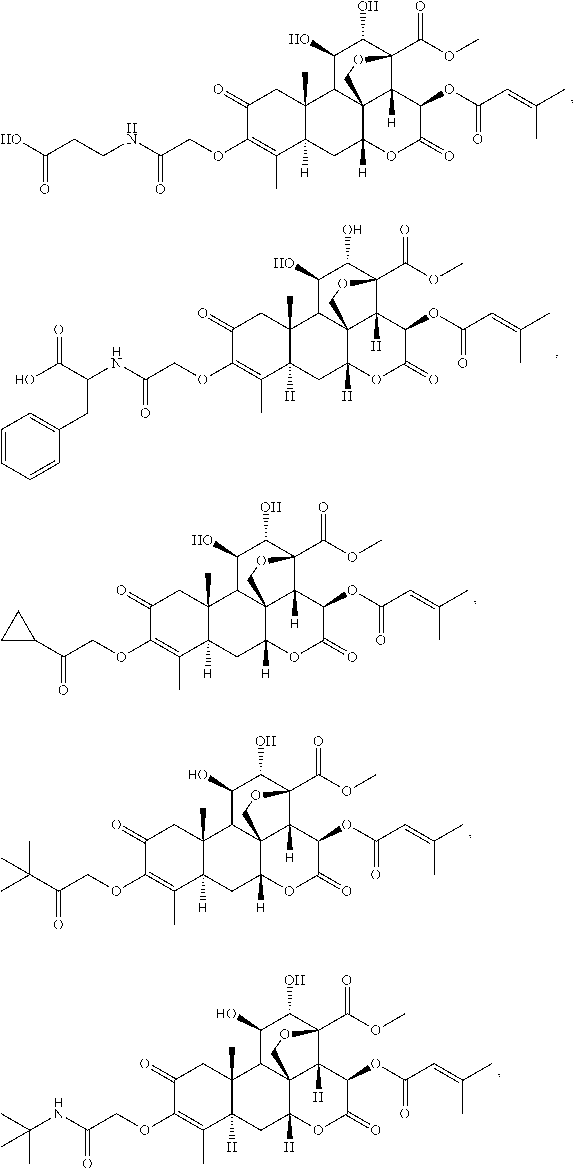

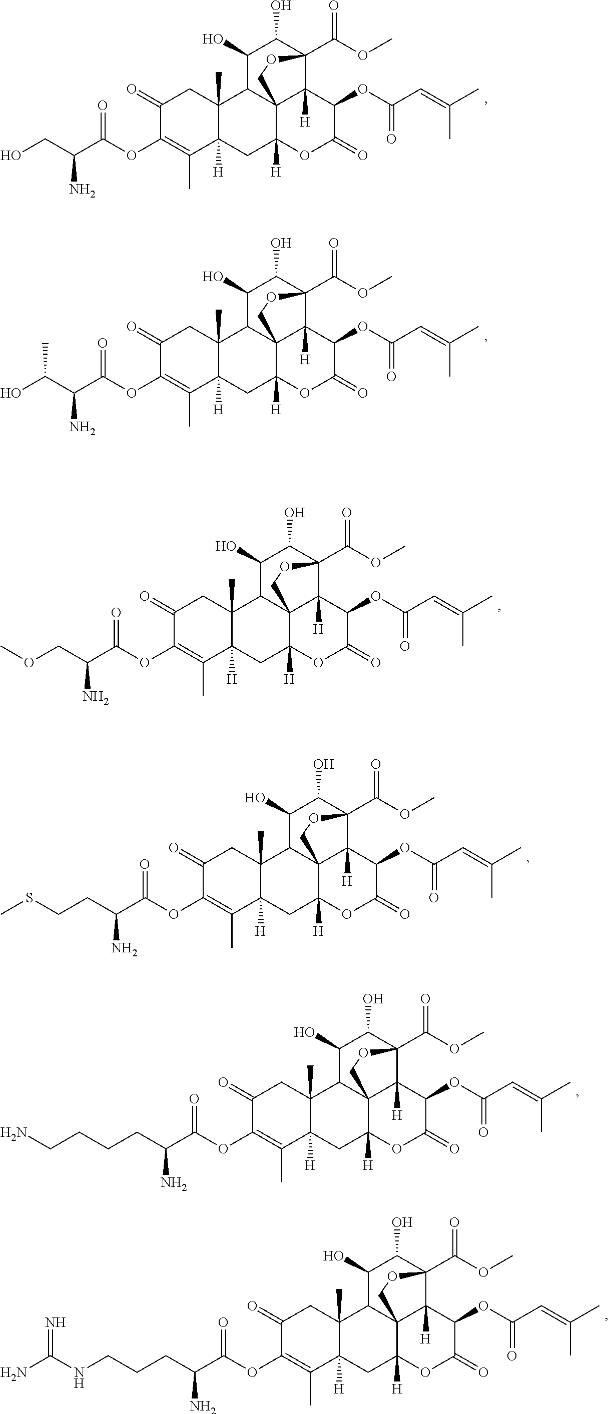

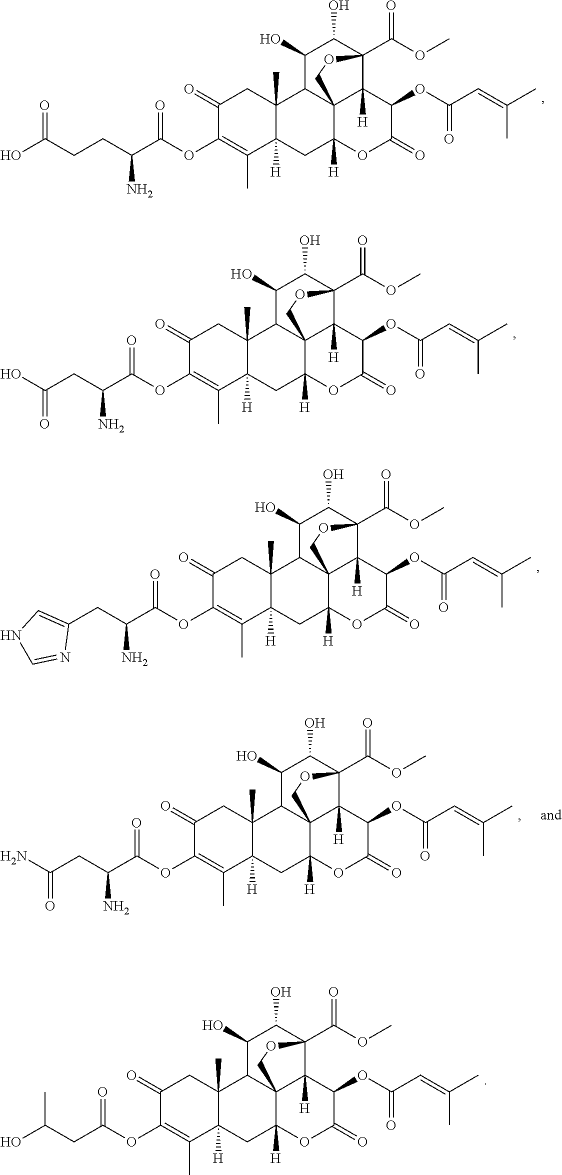

4. A composition comprising a compound of formula (Ib): 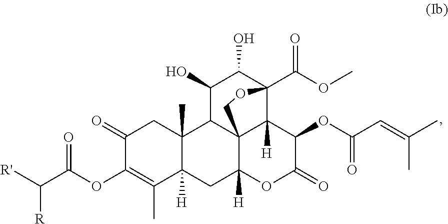
wherein R and R′ are each independently selected from the group consisting of optionally substituted C₁-C₆ alkyl, benzyl, NH₂, NHBoc and H,
or R and R′ combine with the atom to which they are bound to form 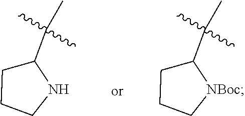 [SMILES: *C1CCCN1 ; *C1CCCN1C(=O)OC(C)(C)C]
or a pharmaceutically acceptable salt, solvate or polymorph thereof.

5. The composition of claim 4, wherein R is C₁-C₃ alkyl optionally substituted with NH₂ or NHBoc.

6. The composition of claim 4, wherein the compound is selected from the group consisting of:

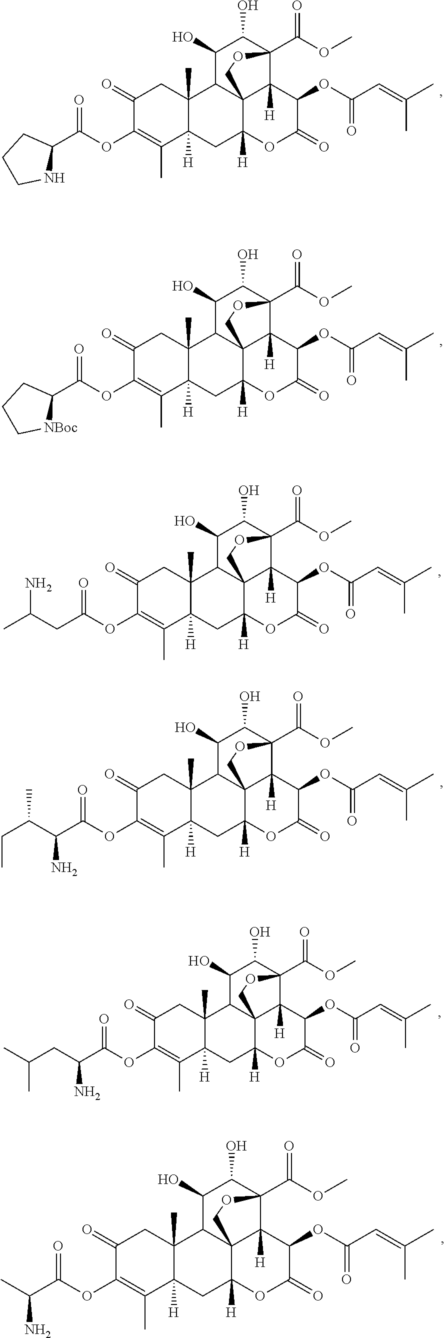

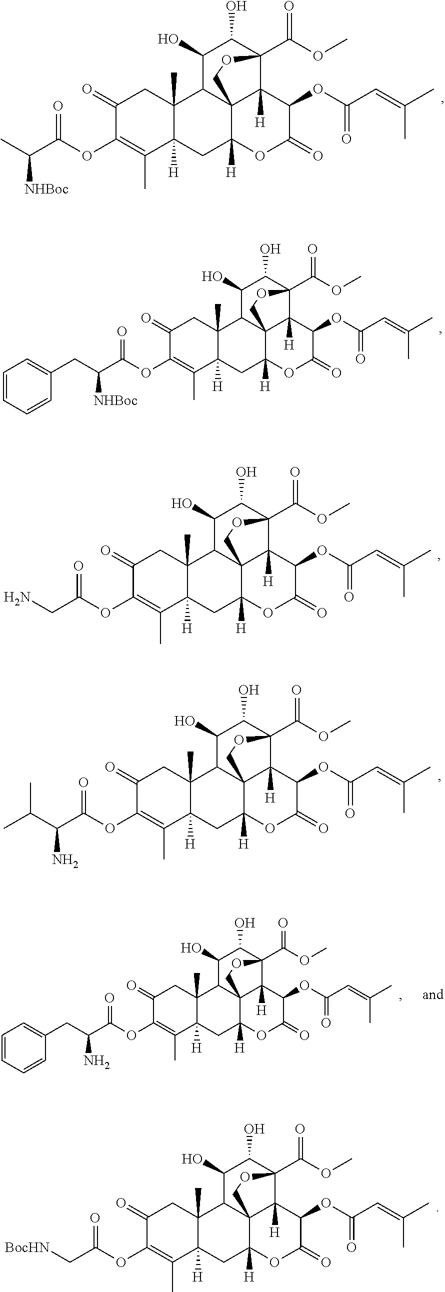

7. The composition of claim 4, wherein the compound is selected from the group consisting of:

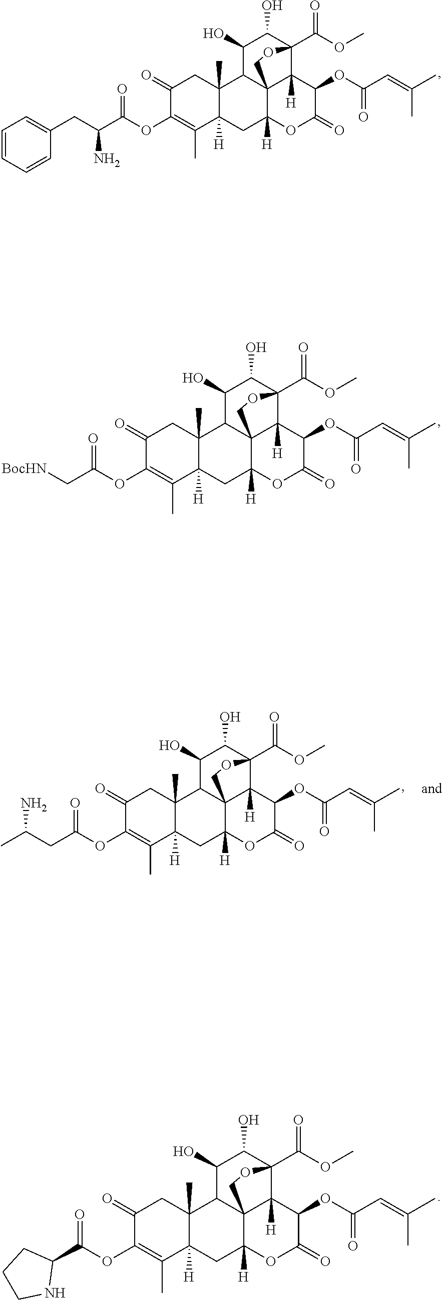

8. A composition comprising a compound of formula (IIa): 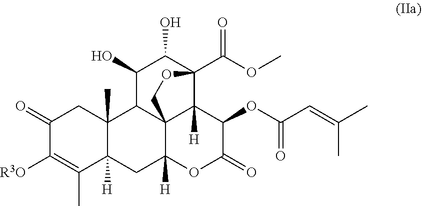
wherein:
R³ is selected from the group consisting of 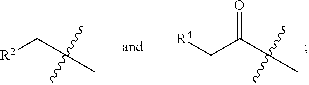
R² is selected from the group consisting of C(═O)N(R^{e})(R^{c}), C(═O)OR^{a}, and C(═O)R^{a};
R⁴ is selected from the group consisting of C₁-C₇ alkyl, C₁-C₇ cycloalkyl, C₁-C₃ aminoalkyl, C₆-C₁₀ aryl, benzyl, OR^{a}, N(R^{a})(R^{b}), SR^{a}, and C(═O)OR^{a},
wherein the C₁-C₇ alkyl, C₁-C₇ alkyl, C₁-C₃ aminoalkyl, C₆-C₁₀ aryl, or benzyl in R⁴ is substituted with at least one substituent selected from the group consisting of OR^{a}, SR^{a}, N(R^{a})(R^{d}), S(═O)₂(C₁-C₃ alkyl), C₃-C₇ cycloalkyl, C₁-C₆ heterocycloalkyl, and halogen;
each occurrence of R^{a} and R^{b} is independently selected from the group consisting of H, C₁-C₆ alkyl, C₃-C₇ cycloalkyl, C(═O)O(C₁-C₆ alkyl), C(═O)(C₁-C₃ alkyl)NH₂, and S(═O)₂(C₁-C₃ alkyl);
each occurrence of R^{c} is independently selected from the group consisting of H, C₁-C₃ alkyl-N(R^{a})(R^{b}), C₁-C₃ alkyl-C(═O)OR^{a}, and CH(CO₂R^{a})CH₂Ph;
each occurrence of R^{d} is independently selected from the group consisting of C₁-C₆ alkyl, C₃-C₇ cycloalkyl, C(═O)C₁-C₆ alkyl, C(═O)O(C₁-C₆ alkyl), C(═O)(C₁-C₃ alkyl)NH₂, and S(═O)₂(C₁-C₃); and
each occurrence of R^{e} is independently selected from the group consisting of H, C₁-C₆ alkyl, and C₃-C₇ cycloalkyl;
or a pharmaceutically acceptable salt, solvate, or polymorph thereof.

9. The composition of claim 8, wherein the compound is selected from the group consisting of:

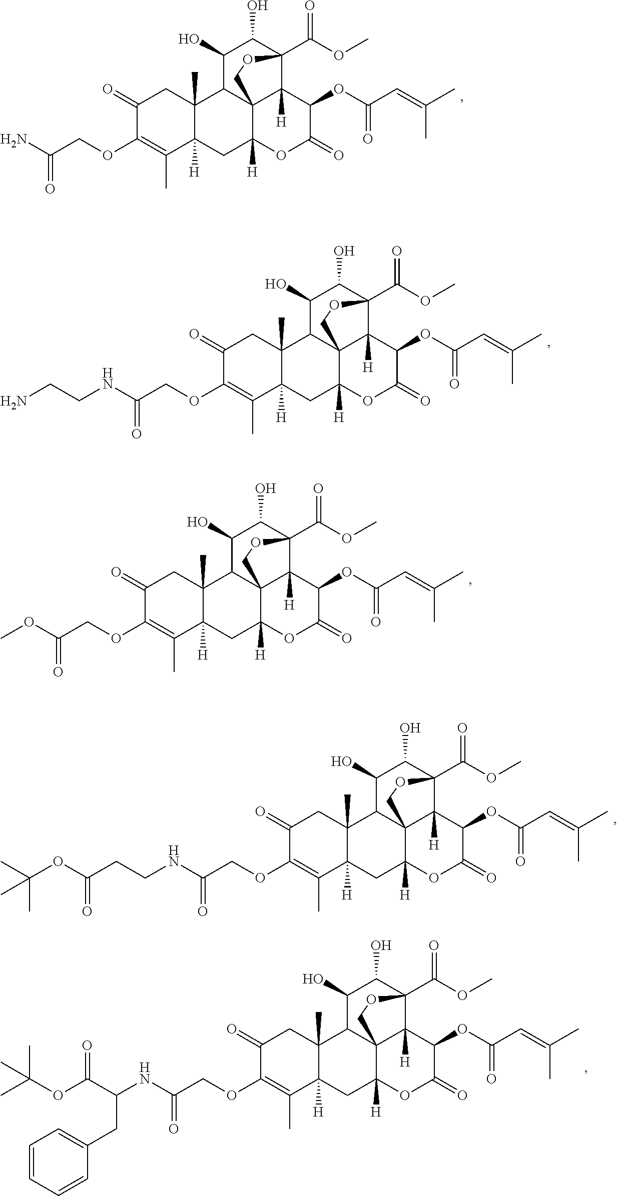

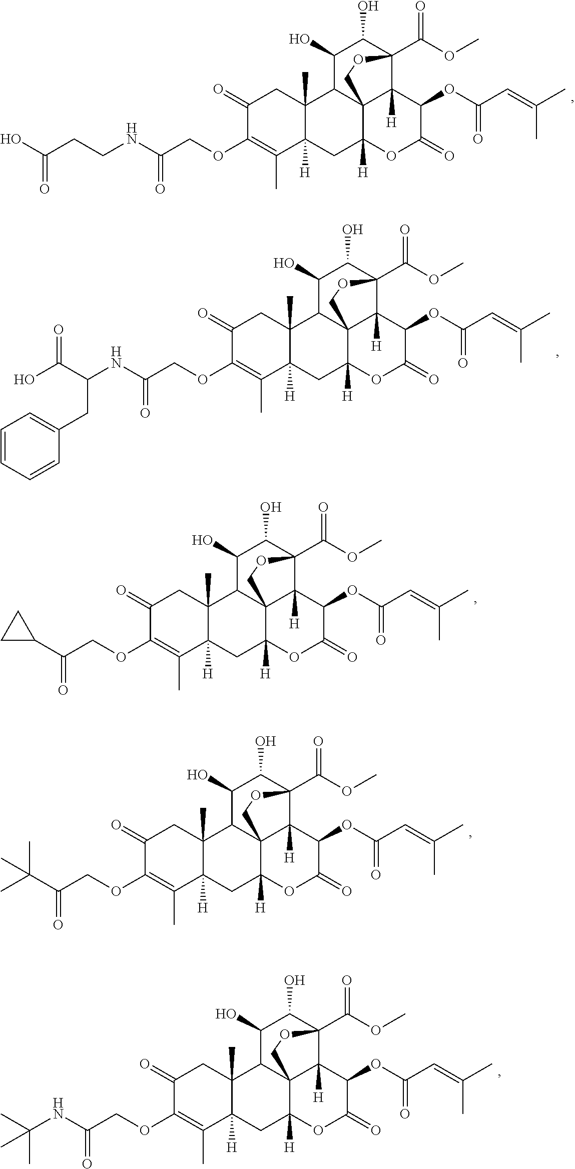

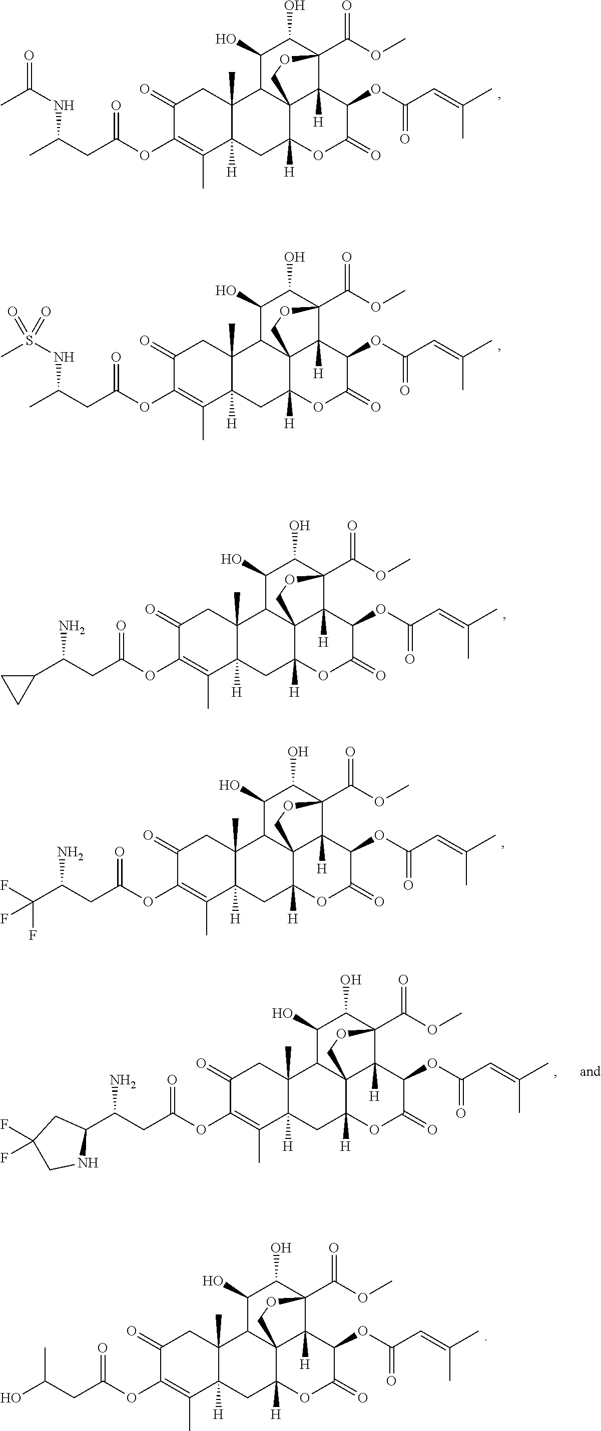

10. A composition comprising a compound of formula (IIb): 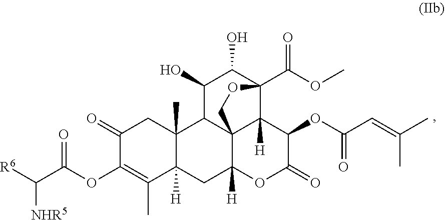
wherein:
R⁵ is selected from the group consisting of H, C₁-C₆ alkyl, and C(═O)O(C₁-C₆ alkyl);
R⁶ is selected from the group consisting of C₁-C₇ alkyl, C₁-C₇ cycloalkyl, C₆-C₁₀ aryl, benzyl, OR^{a}, N(R^{a})(R^{b}), SR^{a}, and C(═O)OR^{a},
wherein the C₁-C₇ alkyl, C₁-C₇ alkyl, C₆-C₁₀ aryl, or benzyl in R⁵ is substituted with at least one substituent selected from the group consisting of C₃-C₇ cycloalkyl, C₁-C₆ heterocycloalkyl, OR^{a}, SR^{a}, N(R^{a})(R^{b}), NR^{a}C(═NR^{a})N(R^{a})(R^{b}), C(═O)OR^{a}, C(═O)N(R^{a})(R^{b}), halogen, and imidazolyl; and
each occurrence of R^{a} and R^{b} is independently selected from the group consisting of H, C₁-C₆ alkyl, C₃-C₇ cycloalkyl, C(═O)C₁-C₆ alkyl, C(═O)O(C₁-C₆ alkyl), C(═O)(C₁-C₃ alkyl)NH₂, and S(═O)₂(C₁-C₃ alkyl);
or a pharmaceutically acceptable salt, solvate, or polymorph thereof.

11. The composition of claim 10, wherein the compound is selected from the group consisting of:

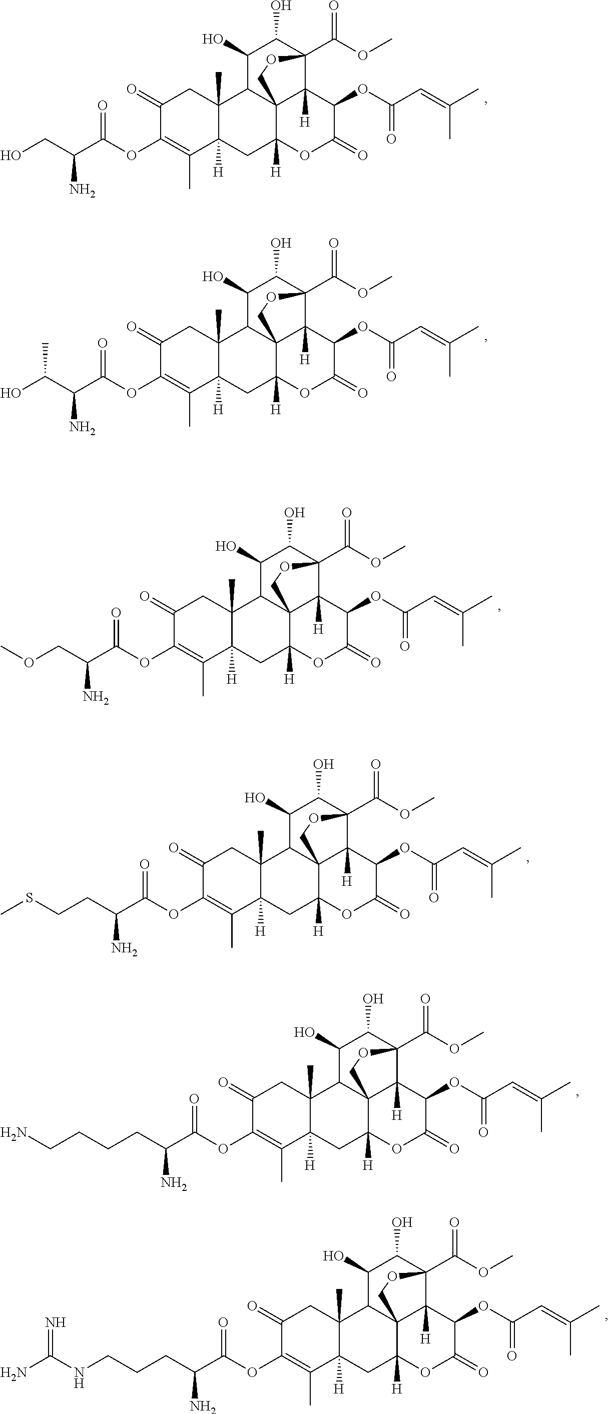

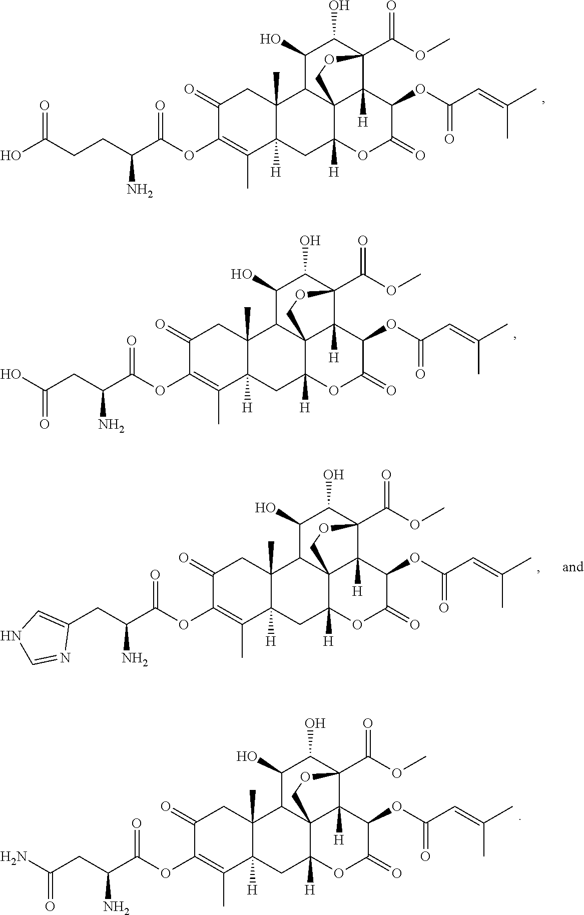

12. The composition of claim 1, further comprising at least one pharmaceutically acceptable carrier.

13. A method of treating a hematologic malignancy in a subject in need thereof, the method comprising administering to the subject a therapeutically effective amount of the composition of claim 12.

14. The method of claim 13, wherein the hematologic malignancy is selected from the group consisting of leukemia and lymphoma.

15. The method of claim 13, wherein the hematologic malignancy is selected from the group consisting of acute myeloid leukemia, Burkitt's lymphoma, B-cell lymphoma, hepatocellular carcinoma, acute promyelocytic leukemia, plasmacytoma, myeloma, chronic myelogenous leukemia, Epstein-Barr Virus (EBV) associated lymphoma, acute lymphoblastic leukemia, acute promyelocytic leukemia, large cell lymphoma, mantle cell lymphoma, and non-Hodgkin's B-cell lymphoma.

16. The method of claim 13, wherein the hematologic malignancy is Epstein-Barr Virus (EBV) associated lymphoma.

17. The method of claim 13, wherein the subject is a mammal.

18. The method of claim 17, wherein the subject is a human.
